(19) United States
(12) Patent Application Publication
(10) Pub. No.: US 2025/0388685 A1
(43) Pub. Date: Dec. 25, 2025

(54) METHODS FOR TREATING CHRONIC RHINOSINUSITIS WITHOUT NASAL POLYPS BY ADMINISTERING AN IL-4R ANTAGONIST

(71) Applicants: SANOFI BIOTECHNOLOGY, Gentilly (FR); REGENERON PHARMACEUTICALS, INC., Tarrytown, NY (US)

(72) Inventors: Elizabeth Laws, Cambridge, MA (US); Leda Mannent, Paris (FR); Lacey Robinson, Cambridge, MA (US); Nikhil Amin, Tarrytown, NY (US)

(21) Appl. No.: 19/178,170

(22) Filed: Apr. 14, 2025

Related U.S. Application Data
(60) Provisional application No. 63/633,944, filed on Apr. 15, 2024.

(30) Foreign Application Priority Data
Apr. 15, 2024 (EP) 24305602.5

(51) Int. Cl. C07K 16/28; A61K 39/00; A61P 11/02

(52) U.S. Cl. CPC C07K 16/2866; A61P 11/02; A61K 2039/505; A61K 2039/545

(57) ABSTRACT

Methods for treating or preventing chronic rhinosinusitis without nasal polyps (CRSsNP) in a subject are provided. Methods comprising administering to a subject in need thereof a therapeutic composition comprising an interleukin-4 receptor (IL-4R) antagonist, such as an anti-IL-4R antibody or antigen-binding fragment thereof, are provided.

### FIELD OF THE INVENTION

This application claims priority to U.S. Provisional Patent Application Ser. No. 63/633,944, filed Apr. 15, 2024, and European Priority Patent Application No. 24305602.5, filed Apr. 15, 2024, the entire disclosures of which are hereby incorporated by reference in their entireties for all purposes.

### FIELD OF THE INVENTION

The disclosure relates to the treatment and/or prevention of chronic rhinosinusitis without nasal polyps (CRSsNP) in a subject in need thereof. The disclosure relates to the administration of an interleukin-4 receptor (IL-4R) antagonist to treat or prevent CRSsNP in a subject in need thereof.

### BACKGROUND

Chronic rhinosinusitis (CRS) is a common disease of the upper respiratory tract and is characterized by chronic inflammation of the sinuses and nasal cavities and can last for at least 12 weeks. CRS patients experience persistent facial pain or pressure, nasal discharge, nasal obstruction, and loss of smell.

Depending on the presence or absence of nasal polyps, CRS has been divided into two types: CRS with nasal polyps (CRSwNP) and CRS without nasal polyps (CRSsNP). Chronic rhinosinusitis without nasal polyps is the most common type of CRS, accounting for approximately two-thirds of the overall CRS population.

Standard medical therapies, such as topical oral corticosteroids (TCS) and oral corticosteroids (OCS), and antibiotics have not provided adequate or lasting control of the symptoms in many patients with CRSsNP, with patients electing to pursue surgery. In patients who have undergone sinonasal surgery, comorbid asthma and peripheral blood eosinophilia are risk factors for the recurrence of CRS post-operatively, suggesting that CRSsNP patients (as indicated by increased peripheral blood eosinophilia and comorbid asthma) may be more at risk for disease recurrence after surgery. Consequently, because of the chronic and unremitting nature of the condition in many patients, CRS is associated with a substantial socioeconomic burden that results from the costs of diagnostic tests, medical and surgical therapies, lost or reduced school and work productivity, and a detrimental impact on physical and emotional health.

Thus, there is a need for therapies that address the disease processes, minimize the need for systemic corticosteroids and their associated side effects, prevent recurrence, prevent complications, and achieve long-lasting disease control.

### SUMMARY

In one aspect, a method for treating a subject having chronic rhinosinusitis without nasal polyps (CRSsNP) is provided, comprising administering to the subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively.

In certain exemplary embodiments, the subject further has asthma, allergic rhinosinusitis, or a combination thereof. In certain exemplary embodiments, the subject does not have asthma, allergic rhinosinusitis, or a combination thereof.

In certain exemplary embodiments, the subject has undergone endoscopic nasal surgery for CRSsNP.

In certain exemplary embodiments, the subject is treated with a background treatment that comprises inhaled corticosteroid (ICS), oral corticosteroid (OCS) or a combination thereof.

In certain exemplary embodiments, the subject has one or more CRSsNP symptoms selected from the group consisting of: 1) nasal congestion/obstruction; 2) anterior rhinorrhea (runny nose); 3) posterior rhinorrhea (post nasal drip); 4) loss of sense of smell; 5) facial pain/pressure; and 6) headache, or any combination thereof.

In certain exemplary embodiments, the subject has a blood eosinophil count of greater than or equal to about 300 cells/μL.

In certain exemplary embodiments, the antibody or antigen-binding fragment thereof is administered to the subject as an initial dose followed by one or more secondary doses.

In certain exemplary embodiments, the initial dose is about 200 to about 300 mg and the one or more secondary doses are each about 200 to about 300 mg. In certain exemplary embodiments, the initial dose is about 300 mg and the one or more secondary doses are each about 300 mg. In certain exemplary embodiments, the initial dose is about 200 mg and the one or more secondary doses are each about 200 mg.

In certain exemplary embodiments, the secondary doses are administered every other week (q2w). In certain exemplary embodiments, the secondary doses are administered every four weeks (q4w).

In certain exemplary embodiments, one or more CRSsNP symptoms are improved in the subject. In certain exemplary embodiments, one or more CRSsNP symptoms include: 1) nasal congestion/obstruction; 2) anterior rhinorrhea (runny nose); 3) posterior rhinorrhea (post nasal drip); 4) loss of sense of smell; 5) facial pain/pressure; and 6) headache, or any combination thereof.

In certain exemplary embodiments, the treatment further reduces one or more of asthma, allergic rhinosinusitis or a combination thereof.

In certain exemplary embodiments, the treatment reduces the level of one or more biomarkers in the subject selected from the group consisting of blood eosinophil (EOS) count, serum immunoglobulin E (IgE) level, eotaxin level, thymus activation-regulated chemokine (TARC) level, IL-5 level, secreted P-glycoprotein level, periostin level and combinations thereof.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2. In certain exemplary embodiments, the antibody is dupilumab

In certain exemplary embodiments, the subject is at least 12 years old. In certain exemplary embodiments, the subject is at least 18 years old.

In certain exemplary embodiments, the antibody or antigen-binding fragment thereof is administered subcutaneously. In certain exemplary embodiments, the antibody or antigen-binding fragment thereof is administered using an autoinjector, a needle and syringe, or a pen. In certain exemplary embodiments, the antibody or antigen-binding fragment thereof is administered using a prefilled device.

In certain exemplary embodiments, the method comprises the step of determining a baseline level of a biomarker selected from the group consisting of IgE, TARC, eotaxin-3, periostin, IL-5, tryptase, eosinophil cationic protein, secreted P-glycoprotein, eosinophils, and any combination thereof in the subject.

In certain exemplary embodiments, uncontrolled CRSsNP is characterized by one or more of: prior sinonasal surgery for chronic rhinosinusitis; prior systemic corticosteroid use for chronic rhinosinusitis; systemic corticosteroid intolerance when previously used for chronic rhinosinusitis; and contraindication of systemic corticosteroid for previous chronic rhinosinusitis treatment. In certain exemplary embodiments, the systemic corticosteroid was administered to the subject at any dose and any duration within two years before treatment with the antibody or antigen-binding fragment thereof.

In another aspect, a method for treating a subject having uncontrolled chronic rhinosinusitis without nasal polyps (CRSsNP) is provided, comprising administering to the subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively.

In certain exemplary embodiments, the subject further has asthma, allergic rhinosinusitis, or a combination thereof. In certain exemplary embodiments, the subject does not have asthma, allergic rhinosinusitis, or a combination thereof.

In certain exemplary embodiments, the subject has undergone endoscopic nasal surgery for CRSsNP.

In certain exemplary embodiments, the subject is treated with a background treatment that comprises inhaled corticosteroid (ICS), oral corticosteroid (OCS) or a combination thereof.

In certain exemplary embodiments, the subject has one or more CRSsNP symptoms selected from the group consisting of: 1) nasal congestion/obstruction; 2) anterior rhinorrhea (runny nose); 3) posterior rhinorrhea (post nasal drip); 4) loss of sense of smell; 5) facial pain/pressure; and 6) headache, or any combination thereof.

In certain exemplary embodiments, the subject has a blood eosinophil count of greater than or equal to about 300 cells/μL.

In certain exemplary embodiments, the antibody or antigen-binding fragment thereof is administered to the subject as an initial dose followed by one or more secondary doses.

In certain exemplary embodiments, the initial dose is about 200 to about 300 mg and the one or more secondary doses are each about 200 to about 300 mg. In certain exemplary embodiments, the initial dose is about 300 mg and the one or more secondary doses are each about 300 mg. In certain exemplary embodiments, the initial dose is about 200 mg and the one or more secondary doses are each about 200 mg.

In certain exemplary embodiments, the secondary doses are administered every other week (q2w). In certain exemplary embodiments, the secondary doses are administered every four weeks (q4w).

In certain exemplary embodiments, one or more CRSsNP symptoms are improved in the subject. In certain exemplary embodiments, one or more CRSsNP symptoms include: 1) nasal congestion/obstruction; 2) anterior rhinorrhea (runny nose); 3) posterior rhinorrhea (post nasal drip); 4) loss of sense of smell; 5) facial pain/pressure; and 6) headache, or any combination thereof.

In certain exemplary embodiments, the treatment further reduces one or more of asthma, allergic rhinosinusitis or a combination thereof.

In certain exemplary embodiments, the treatment reduces the level of one or more biomarkers in the subject selected from the group consisting of blood eosinophil (EOS) count, serum immunoglobulin E (IgE) level, eotaxin level, thymus activation-regulated chemokine (TARC) level, IL-5 level, secreted P-glycoprotein level, periostin level and combinations thereof.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2. In certain exemplary embodiments, the antibody is dupilumab

In certain exemplary embodiments, the subject is at least 12 years old. In certain exemplary embodiments, the subject is at least 18 years old.

In certain exemplary embodiments, the antibody or antigen-binding fragment thereof is administered subcutaneously. In certain exemplary embodiments, the antibody or antigen-binding fragment thereof is administered using an autoinjector, a needle and syringe, or a pen. In certain exemplary embodiments, the antibody or antigen-binding fragment thereof is administered using a prefilled device.

In certain exemplary embodiments, the method comprises the step of determining a baseline level of a biomarker selected from the group consisting of IgE, TARC, eotaxin-3, periostin, IL-5, tryptase, eosinophil cationic protein, secreted P-glycoprotein, eosinophils, and any combination thereof in the subject.

In certain exemplary embodiments, uncontrolled CRSsNP is characterized by one or more of: prior sinonasal surgery for chronic rhinosinusitis; prior systemic corticosteroid use for chronic rhinosinusitis; systemic corticosteroid intolerance when previously used for chronic rhinosinusitis; and contraindication of systemic corticosteroid for previous chronic rhinosinusitis treatment. In certain exemplary embodiments, the systemic corticosteroid was administered to the subject at any dose and any duration within two years before treatment with the antibody or antigen-binding fragment thereof.

In another aspect, a method for treating a subject having chronic rhinosinusitis without nasal polyps (CRSsNP) with Type 2 inflammation is provided, comprising administering to the subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively.

In certain exemplary embodiments, the subject further has asthma, allergic rhinosinusitis, or a combination thereof. In certain exemplary embodiments, the subject does not have asthma, allergic rhinosinusitis, or a combination thereof.

In certain exemplary embodiments, the subject has undergone endoscopic nasal surgery for CRSsNP.

In certain exemplary embodiments, the subject is treated with a background treatment that comprises inhaled corticosteroid (ICS), oral corticosteroid (OCS) or a combination thereof.

In certain exemplary embodiments, the subject has one or more CRSsNP symptoms selected from the group consisting of: 1) nasal congestion/obstruction; 2) anterior rhinorrhea (runny nose); 3) posterior rhinorrhea (post nasal drip); 4) loss of sense of smell; 5) facial pain/pressure; and 6) headache, or any combination thereof.

In certain exemplary embodiments, the subject has a blood eosinophil count of greater than or equal to about 300 cells/μL.

In certain exemplary embodiments, the antibody or antigen-binding fragment thereof is administered to the subject as an initial dose followed by one or more secondary doses.

In certain exemplary embodiments, the initial dose is about 200 to about 300 mg and the one or more secondary doses are each about 200 to about 300 mg. In certain exemplary embodiments, the initial dose is about 300 mg and the one or more secondary doses are each about 300 mg. In certain exemplary embodiments, the initial dose is about 200 mg and the one or more secondary doses are each about 200 mg.

In certain exemplary embodiments, the secondary doses are administered every other week (q2w). In certain exemplary embodiments, the secondary doses are administered every four weeks (q4w).

In certain exemplary embodiments, one or more CRSsNP symptoms are improved in the subject. In certain exemplary embodiments, one or more CRSsNP symptoms include: 1) nasal congestion/obstruction; 2) anterior rhinorrhea (runny nose); 3) posterior rhinorrhea (post nasal drip); 4) loss of sense of smell; 5) facial pain/pressure; and 6) headache, or any combination thereof.

In certain exemplary embodiments, the treatment further reduces one or more of asthma, allergic rhinosinusitis or a combination thereof.

In certain exemplary embodiments, the treatment reduces the level of one or more biomarkers in the subject selected from the group consisting of blood eosinophil (EOS) count, serum immunoglobulin E (IgE) level, eotaxin level, thymus activation-regulated chemokine (TARC) level, IL-5 level, secreted P-glycoprotein level, periostin level and combinations thereof.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2. In certain exemplary embodiments, the antibody is dupilumab

In certain exemplary embodiments, the subject is at least 12 years old. In certain exemplary embodiments, the subject is at least 18 years old.

In certain exemplary embodiments, the antibody or antigen-binding fragment thereof is administered subcutaneously. In certain exemplary embodiments, the antibody or antigen-binding fragment thereof is administered using an autoinjector, a needle and syringe, or a pen. In certain exemplary embodiments, the antibody or antigen-binding fragment thereof is administered using a prefilled device.

In certain exemplary embodiments, the method comprises the step of determining a baseline level of a biomarker selected from the group consisting of IgE, TARC, eotaxin-3, periostin, IL-5, tryptase, eosinophil cationic protein, secreted P-glycoprotein, eosinophils, and any combination thereof in the subject.

In certain exemplary embodiments, uncontrolled CRSsNP is characterized by one or more of: prior sinonasal surgery for chronic rhinosinusitis; prior systemic corticosteroid use for chronic rhinosinusitis; systemic corticosteroid intolerance when previously used for chronic rhinosinusitis; and contraindication of systemic corticosteroid for previous chronic rhinosinusitis treatment. In certain exemplary embodiments, the systemic corticosteroid was administered to the subject at any dose and any duration within two years before treatment with the antibody or antigen-binding fragment thereof.

In another aspect, a method for treating a subject having chronic rhinosinusitis without nasal polyps (CRSsNP) not adequately controlled on background treatment is provided, comprising administering to the subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively.

In certain exemplary embodiments, the subject further has asthma, allergic rhinosinusitis, or a combination thereof. In certain exemplary embodiments, the subject does not have asthma, allergic rhinosinusitis, or a combination thereof.

In certain exemplary embodiments, the subject has undergone endoscopic nasal surgery for CRSsNP.

In certain exemplary embodiments, the subject is treated with a background treatment that comprises inhaled corticosteroid (ICS), oral corticosteroid (OCS) or a combination thereof.

In certain exemplary embodiments, the subject has one or more CRSsNP symptoms selected from the group consisting of: 1) nasal congestion/obstruction; 2) anterior rhinorrhea (runny nose); 3) posterior rhinorrhea (post nasal drip); 4) loss of sense of smell; 5) facial pain/pressure; and 6) headache, or any combination thereof.

In certain exemplary embodiments, the subject has a blood eosinophil count of greater than or equal to about 300 cells/μL.

In certain exemplary embodiments, the antibody or antigen-binding fragment thereof is administered to the subject as an initial dose followed by one or more secondary doses.

In certain exemplary embodiments, the initial dose is about 200 to about 300 mg and the one or more secondary doses are each about 200 to about 300 mg. In certain exemplary embodiments, the initial dose is about 300 mg and the one or more secondary doses are each about 300 mg. In certain exemplary embodiments, the initial dose is about 200 mg and the one or more secondary doses are each about 200 mg.

In certain exemplary embodiments, the secondary doses are administered every other week (q2w). In certain exemplary embodiments, the secondary doses are administered every four weeks (q4w).

In certain exemplary embodiments, one or more CRSsNP symptoms are improved in the subject. In certain exemplary embodiments, one or more CRSsNP symptoms include: 1) nasal congestion/obstruction; 2) anterior rhinorrhea (runny nose); 3) posterior rhinorrhea (post nasal drip); 4) loss of sense of smell; 5) facial pain/pressure; and 6) headache, or any combination thereof.

In certain exemplary embodiments, the treatment further reduces one or more of asthma, allergic rhinosinusitis or a combination thereof.

In certain exemplary embodiments, the treatment reduces the level of one or more biomarkers in the subject selected from the group consisting of blood eosinophil (EOS) count, serum immunoglobulin E (IgE) level, eotaxin level, thymus activation-regulated chemokine (TARC) level, IL-5 level, secreted P-glycoprotein level, periostin level and combinations thereof.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2. In certain exemplary embodiments, the antibody is dupilumab

In certain exemplary embodiments, the subject is at least 12 years old. In certain exemplary embodiments, the subject is at least 18 years old.

In certain exemplary embodiments, the antibody or antigen-binding fragment thereof is administered subcutaneously. In certain exemplary embodiments, the antibody or antigen-binding fragment thereof is administered using an autoinjector, a needle and syringe, or a pen. In certain exemplary embodiments, the antibody or antigen-binding fragment thereof is administered using a prefilled device.

In certain exemplary embodiments, the method comprises the step of determining a baseline level of a biomarker selected from the group consisting of IgE, TARC, eotaxin-3, periostin, IL-5, tryptase, eosinophil cationic protein, secreted P-glycoprotein, eosinophils, and any combination thereof in the subject.

In certain exemplary embodiments, uncontrolled CRSsNP is characterized by one or more of: prior sinonasal surgery for chronic rhinosinusitis; prior systemic corticosteroid use for chronic rhinosinusitis; systemic corticosteroid intolerance when previously used for chronic rhinosinusitis; and contraindication of systemic corticosteroid for previous chronic rhinosinusitis treatment. In certain exemplary embodiments, the systemic corticosteroid was administered to the subject at any dose and any duration within two years before treatment with the antibody or antigen-binding fragment thereof.

On another aspect, a method for treating a subject having uncontrolled chronic rhinosinusitis without nasal polyps (CRSsNP) with Type 2 inflammation is provided, comprising administering to the subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively.

In certain exemplary embodiments, the subject further has asthma, allergic rhinosinusitis, or a combination thereof. In certain exemplary embodiments, the subject does not have asthma, allergic rhinosinusitis, or a combination thereof.

In certain exemplary embodiments, the subject has undergone endoscopic nasal surgery for CRSsNP.

In certain exemplary embodiments, the subject is treated with a background treatment that comprises inhaled corticosteroid (ICS), oral corticosteroid (OCS) or a combination thereof.

In certain exemplary embodiments, the subject has one or more CRSsNP symptoms selected from the group consisting of: 1) nasal congestion/obstruction; 2) anterior rhinorrhea (runny nose); 3) posterior rhinorrhea (post nasal drip); 4) loss of sense of smell; 5) facial pain/pressure; and 6) headache, or any combination thereof.

In certain exemplary embodiments, the subject has a blood eosinophil count of greater than or equal to about 300 cells/μL.

In certain exemplary embodiments, the antibody or antigen-binding fragment thereof is administered to the subject as an initial dose followed by one or more secondary doses.

In certain exemplary embodiments, the initial dose is about 200 to about 300 mg and the one or more secondary doses are each about 200 to about 300 mg. In certain exemplary embodiments, the initial dose is about 300 mg and the one or more secondary doses are each about 300 mg. In certain exemplary embodiments, the initial dose is about 200 mg and the one or more secondary doses are each about 200 mg.

In certain exemplary embodiments, the secondary doses are administered every other week (q2w). In certain exemplary embodiments, the secondary doses are administered every four weeks (q4w).

In certain exemplary embodiments, one or more CRSsNP symptoms are improved in the subject. In certain exemplary embodiments, one or more CRSsNP symptoms include: 1) nasal congestion/obstruction; 2) anterior rhinorrhea (runny nose); 3) posterior rhinorrhea (post nasal drip); 4) loss of sense of smell; 5) facial pain/pressure; and 6) headache, or any combination thereof.

In certain exemplary embodiments, the treatment further reduces one or more of asthma, allergic rhinosinusitis or a combination thereof.

In certain exemplary embodiments, the treatment reduces the level of one or more biomarkers in the subject selected from the group consisting of blood eosinophil (EOS) count, serum immunoglobulin E (IgE) level, eotaxin level, thymus activation-regulated chemokine (TARC) level, IL-5 level, secreted P-glycoprotein level, periostin level and combinations thereof.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2. In certain exemplary embodiments, the antibody is dupilumab

In certain exemplary embodiments, the subject is at least 12 years old. In certain exemplary embodiments, the subject is at least 18 years old.

In certain exemplary embodiments, the antibody or antigen-binding fragment thereof is administered subcutaneously. In certain exemplary embodiments, the antibody or antigen-binding fragment thereof is administered using an autoinjector, a needle and syringe, or a pen. In certain exemplary embodiments, the antibody or antigen-binding fragment thereof is administered using a prefilled device.

In certain exemplary embodiments, the method comprises the step of determining a baseline level of a biomarker selected from the group consisting of IgE, TARC, eotaxin-3, periostin, IL-5, tryptase, eosinophil cationic protein, secreted P-glycoprotein, eosinophils, and any combination thereof in the subject.

In certain exemplary embodiments, uncontrolled CRSsNP is characterized by one or more of: prior sinonasal surgery for chronic rhinosinusitis; prior systemic corticosteroid use for chronic rhinosinusitis; systemic corticosteroid intolerance when previously used for chronic rhinosinusitis; and contraindication of systemic corticosteroid for previous chronic rhinosinusitis treatment. In certain exemplary embodiments, the systemic corticosteroid was administered to the subject at any dose and any duration within two years before treatment with the antibody or antigen-binding fragment thereof.

In another aspect, a method for treating a subject having uncontrolled chronic rhinosinusitis without nasal polyps (CRSsNP) is provided, comprising selecting a subject having uncontrolled CRSsNP, and administering to the subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively.

In certain exemplary embodiments, the subject further has asthma, allergic rhinosinusitis, or a combination thereof. In certain exemplary embodiments, the subject does not have asthma, allergic rhinosinusitis, or a combination thereof.

In certain exemplary embodiments, the subject has undergone endoscopic nasal surgery for CRSsNP.

In certain exemplary embodiments, the subject is treated with a background treatment that comprises inhaled corticosteroid (ICS), oral corticosteroid (OCS) or a combination thereof.

In certain exemplary embodiments, the subject has one or more CRSsNP symptoms selected from the group consisting of: 1) nasal congestion/obstruction; 2) anterior rhinorrhea (runny nose); 3) posterior rhinorrhea (post nasal drip); 4) loss of sense of smell; 5) facial pain/pressure; and 6) headache, or any combination thereof.

In certain exemplary embodiments, the subject has a blood eosinophil count of greater than or equal to about 300 cells/μL.

In certain exemplary embodiments, the antibody or antigen-binding fragment thereof is administered to the subject as an initial dose followed by one or more secondary doses.

In certain exemplary embodiments, the initial dose is about 200 to about 300 mg and the one or more secondary doses are each about 200 to about 300 mg. In certain exemplary embodiments, the initial dose is about 300 mg and the one or more secondary doses are each about 300 mg. In certain exemplary embodiments, the initial dose is about 200 mg and the one or more secondary doses are each about 200 mg.

In certain exemplary embodiments, the secondary doses are administered every other week (q2w). In certain exemplary embodiments, the secondary doses are administered every four weeks (q4w).

In certain exemplary embodiments, one or more CRSsNP symptoms are improved in the subject. In certain exemplary embodiments, one or more CRSsNP symptoms include: 1) nasal congestion/obstruction; 2) anterior rhinorrhea (runny nose); 3) posterior rhinorrhea (post nasal drip); 4) loss of sense of smell; 5) facial pain/pressure; and 6) headache, or any combination thereof.

In certain exemplary embodiments, the treatment further reduces one or more of asthma, allergic rhinosinusitis or a combination thereof.

In certain exemplary embodiments, the treatment reduces the level of one or more biomarkers in the subject selected from the group consisting of blood eosinophil (EOS) count, serum immunoglobulin E (IgE) level, eotaxin level, thymus activation-regulated chemokine (TARC) level, IL-5 level, secreted P-glycoprotein level, periostin level and combinations thereof.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2. In certain exemplary embodiments, the antibody is dupilumab

In certain exemplary embodiments, the subject is at least 12 years old. In certain exemplary embodiments, the subject is at least 18 years old.

In certain exemplary embodiments, the antibody or antigen-binding fragment thereof is administered subcutaneously. In certain exemplary embodiments, the antibody or antigen-binding fragment thereof is administered using an autoinjector, a needle and syringe, or a pen. In certain exemplary embodiments, the antibody or antigen-binding fragment thereof is administered using a prefilled device.

In certain exemplary embodiments, the method comprises the step of determining a baseline level of a biomarker selected from the group consisting of IgE, TARC, eotaxin-3, periostin, IL-5, tryptase, eosinophil cationic protein, secreted P-glycoprotein, eosinophils, and any combination thereof in the subject.

In certain exemplary embodiments, uncontrolled CRSsNP is characterized by one or more of: prior sinonasal surgery for chronic rhinosinusitis; prior systemic corticosteroid use for chronic rhinosinusitis; systemic corticosteroid intolerance when previously used for chronic rhinosinusitis; and contraindication of systemic corticosteroid for previous chronic rhinosinusitis treatment. In certain exemplary embodiments, the systemic corticosteroid was administered to the subject at any dose and any duration within two years before treatment with the antibody or antigen-binding fragment thereof.

In another aspect, a method for reducing systemic corticosteroid (SCS) use in a subject having chronic rhinosinusitis without nasal polyps (CRSsNP) is provided, comprising administering to the subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively.

In certain exemplary embodiments, the subject further has asthma, allergic rhinosinusitis, or a combination thereof. In certain exemplary embodiments, the subject does not have asthma, allergic rhinosinusitis, or a combination thereof.

In certain exemplary embodiments, the subject has undergone endoscopic nasal surgery for CRSsNP.

In certain exemplary embodiments, the subject is treated with a background treatment that comprises inhaled corticosteroid (ICS), oral corticosteroid (OCS) or a combination thereof.

In certain exemplary embodiments, the subject has one or more CRSsNP symptoms selected from the group consisting of: 1) nasal congestion/obstruction; 2) anterior rhinorrhea (runny nose); 3) posterior rhinorrhea (post nasal drip); 4) loss of sense of smell; 5) facial pain/pressure; and 6) headache, or any combination thereof.

In certain exemplary embodiments, the subject has a blood eosinophil count of greater than or equal to about 300 cells/μL.

In certain exemplary embodiments, the antibody or antigen-binding fragment thereof is administered to the subject as an initial dose followed by one or more secondary doses.

In certain exemplary embodiments, the initial dose is about 200 to about 300 mg and the one or more secondary doses are each about 200 to about 300 mg. In certain exemplary embodiments, the initial dose is about 300 mg and the one or more secondary doses are each about 300 mg. In certain exemplary embodiments, the initial dose is about 200 mg and the one or more secondary doses are each about 200 mg.

In certain exemplary embodiments, the secondary doses are administered every other week (q2w). In certain exemplary embodiments, the secondary doses are administered every four weeks (q4w).

In certain exemplary embodiments, one or more CRSsNP symptoms are improved in the subject. In certain exemplary embodiments, one or more CRSsNP symptoms include: 1) nasal congestion/obstruction; 2) anterior rhinorrhea (runny nose); 3) posterior rhinorrhea (post nasal drip); 4) loss of sense of smell; 5) facial pain/pressure; and 6) headache, or any combination thereof.

In certain exemplary embodiments, the treatment further reduces one or more of asthma, allergic rhinosinusitis or a combination thereof.

In certain exemplary embodiments, the treatment reduces the level of one or more biomarkers in the subject selected from the group consisting of blood eosinophil (EOS) count, serum immunoglobulin E (IgE) level, eotaxin level, thymus activation-regulated chemokine (TARC) level, IL-5 level, secreted P-glycoprotein level, periostin level and combinations thereof.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2. In certain exemplary embodiments, the antibody is dupilumab

In certain exemplary embodiments, the subject is at least 12 years old. In certain exemplary embodiments, the subject is at least 18 years old.

In certain exemplary embodiments, the antibody or antigen-binding fragment thereof is administered subcutaneously. In certain exemplary embodiments, the antibody or antigen-binding fragment thereof is administered using an autoinjector, a needle and syringe, or a pen. In certain exemplary embodiments, the antibody or antigen-binding fragment thereof is administered using a prefilled device.

In certain exemplary embodiments, the method comprises the step of determining a baseline level of a biomarker selected from the group consisting of IgE, TARC, eotaxin-3, periostin, IL-5, tryptase, eosinophil cationic protein, secreted P-glycoprotein, eosinophils, and any combination thereof in the subject.

In certain exemplary embodiments, uncontrolled CRSsNP is characterized by one or more of: prior sinonasal surgery for chronic rhinosinusitis; prior systemic corticosteroid use for chronic rhinosinusitis; systemic corticosteroid intolerance when previously used for chronic rhinosinusitis; and contraindication of systemic corticosteroid for previous chronic rhinosinusitis treatment. In certain exemplary embodiments, the systemic corticosteroid was administered to the subject at any dose and any duration within two years before treatment with the antibody or antigen-binding fragment thereof.

In another aspect, a method for reducing oral corticosteroid (OCS) use in a subject having chronic rhinosinusitis without nasal polyps (CRSsNP) is provided, comprising administering to the subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively.

In certain exemplary embodiments, the subject further has asthma, allergic rhinosinusitis, or a combination thereof. In certain exemplary embodiments, the subject does not have asthma, allergic rhinosinusitis, or a combination thereof.

In certain exemplary embodiments, the subject has undergone endoscopic nasal surgery for CRSsNP.

In certain exemplary embodiments, the subject is treated with a background treatment that comprises inhaled corticosteroid (ICS), oral corticosteroid (OCS) or a combination thereof.

In certain exemplary embodiments, the subject has one or more CRSsNP symptoms selected from the group consisting of: 1) nasal congestion/obstruction; 2) anterior rhinorrhea (runny nose); 3) posterior rhinorrhea (post nasal drip); 4) loss of sense of smell; 5) facial pain/pressure; and 6) headache, or any combination thereof.

In certain exemplary embodiments, the subject has a blood eosinophil count of greater than or equal to about 300 cells/μL.

In certain exemplary embodiments, the antibody or antigen-binding fragment thereof is administered to the subject as an initial dose followed by one or more secondary doses.

In certain exemplary embodiments, the initial dose is about 200 to about 300 mg and the one or more secondary doses are each about 200 to about 300 mg. In certain exemplary embodiments, the initial dose is about 300 mg and the one or more secondary doses are each about 300 mg. In certain exemplary embodiments, the initial dose is about 200 mg and the one or more secondary doses are each about 200 mg.

In certain exemplary embodiments, the secondary doses are administered every other week (q2w). In certain exemplary embodiments, the secondary doses are administered every four weeks (q4w).

In certain exemplary embodiments, one or more CRSsNP symptoms are improved in the subject. In certain exemplary embodiments, one or more CRSsNP symptoms include: 1) nasal congestion/obstruction; 2) anterior rhinorrhea (runny nose); 3) posterior rhinorrhea (post nasal drip); 4) loss of sense of smell; 5) facial pain/pressure; and 6) headache, or any combination thereof.

In certain exemplary embodiments, the treatment further reduces one or more of asthma, allergic rhinosinusitis or a combination thereof.

In certain exemplary embodiments, the treatment reduces the level of one or more biomarkers in the subject selected from the group consisting of blood eosinophil (EOS) count, serum immunoglobulin E (IgE) level, eotaxin level, thymus activation-regulated chemokine (TARC) level, IL-5 level, secreted P-glycoprotein level, periostin level and combinations thereof.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2. In certain exemplary embodiments, the antibody is dupilumab

In certain exemplary embodiments, the subject is at least 12 years old. In certain exemplary embodiments, the subject is at least 18 years old.

In certain exemplary embodiments, the antibody or antigen-binding fragment thereof is administered subcutaneously. In certain exemplary embodiments, the antibody or antigen-binding fragment thereof is administered using an autoinjector, a needle and syringe, or a pen. In certain exemplary embodiments, the antibody or antigen-binding fragment thereof is administered using a prefilled device.

In certain exemplary embodiments, the method comprises the step of determining a baseline level of a biomarker selected from the group consisting of IgE, TARC, eotaxin-3, periostin, IL-5, tryptase, eosinophil cationic protein, secreted P-glycoprotein, eosinophils, and any combination thereof in the subject.

In certain exemplary embodiments, uncontrolled CRSsNP is characterized by one or more of: prior sinonasal surgery for chronic rhinosinusitis; prior systemic corticosteroid use for chronic rhinosinusitis; systemic corticosteroid intolerance when previously used for chronic rhinosinusitis; and contraindication of systemic corticosteroid for previous chronic rhinosinusitis treatment. In certain exemplary embodiments, the systemic corticosteroid was administered to the subject at any dose and any duration within two years before treatment with the antibody or antigen-binding fragment thereof.

In another aspect, a method for treating a subject having uncontrolled chronic rhinosinusitis without nasal polyps (CRSsNP), wherein the subject has a blood eosinophil level of ≥300 cells/μL is provided, comprising administering to the subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively.

In certain exemplary embodiments, the subject has one or more symptoms of CRSsNP selected from the group consisting of: bilateral inflammation of paranasal sinuses; a Lund-Mackay (LMK) score of ≥8; a sinus total symptom (sTSS) score of ≥5; prior sinonasal surgery for chronic rhinosinusitis (CRS); and prior systemic corticosteroid (SCS) use for CRS.

In certain exemplary embodiments, the antibody or antigen-binding fragment thereof is administered to the subject as an initial dose followed by one or more secondary doses. In certain exemplary embodiments, the initial dose is about 300 mg and the one or more secondary doses are each about 300 mg.

In certain exemplary embodiments, the antibody or antigen-binding fragment thereof is administered every two weeks (Q2W).

In certain exemplary embodiments, one or more CRSsNP symptoms are improved in the subject. In certain exemplary embodiments, the one or more CRSsNP symptoms are selected from the group consisting of LMK score, sTSS score, University of Pennsylvania Smell Identification Test (UPSIT) score, and Sinonasal Outcome Test-22 (SNOT-22) score.

In certain exemplary embodiments, one or more biomarkers of Type 2 inflammation are decreased in the subject. In certain exemplary embodiments, the one or more biomarkers are selected from the group consisting of immunoglobulin E (IgE), eotaxin-3, and thymus activation regulated chemokine (TARC).

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2. In certain exemplary embodiments, the antibody is dupilumab.

In certain exemplary embodiments, the subject is at least 18 years old.

In certain exemplary embodiments, the antibody or antigen-binding fragment thereof is administered subcutaneously. In certain exemplary embodiments, the antibody or antigen-binding fragment thereof is administered using an autoinjector, a needle and syringe, or a pen. In certain exemplary embodiments, the antibody or antigen-binding fragment thereof is administered using a prefilled device.

In certain exemplary embodiments, uncontrolled CRSsNP is characterized by one or more of: prior sinonasal surgery for chronic rhinosinusitis; prior systemic corticosteroid use for chronic rhinosinusitis; systemic corticosteroid intolerance when previously used for chronic rhinosinusitis; and contraindication of systemic corticosteroid for previous chronic rhinosinusitis treatment. In certain exemplary embodiments, the systemic corticosteroid was administered to the subject at any dose and any duration within two years before treatment with the antibody or antigen-binding fragment thereof.

### BRIEF DESCRIPTION OF THE FIGURES

The foregoing and other features and advantages of the disclosure will be more fully understood from the following detailed description of illustrative embodiments taken in conjunction with the accompanying drawings.

FIG. 1 schematically depicts the overview of the study design of Example 1. The study is a multinational, randomized, double-blind, placebo-controlled, 52-week phase 2/3 2-part study to assess the efficacy, safety, and tolerability of dupilumab in patients with CRSsNP.

FIG. 2A-FIG. 2G shows the schedule of activities for the randomized, placebo-controlled study to assess the efficacy, safety, and tolerability of dupilumab in patients with CRSsNP and as described in Example 1.

FIG. 3 schematically depicts the ORION Phase 2 study design of Example 2. ¹Stratified by screening blood eosinophil count (< or ≥300 cells/μl), background INCS use, region. Participants without comorbid asthma capped at 70% of the randomized population. ²Treatment for all participants stopped when last patient in (LPI) reached 24 weeks of treatment.

FIG. 4 depicts a Lund-Mackay (LMK) radiographic scan and scoring table.

FIG. 5 is a table depicting baseline demographics, disease characteristics and biomarker data comparing CRSsNP and CRSwNP. ¹Inclusion criteria for SINUS studies included SCS in prior 2 years or sinonasal surgery.

FIG. 6 graphically depicts that dupilumab led to a reduction in LMK score in CRSsNP patients with either EOS levels of ≥300 cells/μL or EOS levels of <300 cells/μL. LMK is a radiographic score assessing each sinus and osteomeatal complex on each side for opacification, with a total score 0-24 (a higher score indicates worse radiographic features). The minimal clinically important differences (MCID) score is −4.

FIG. 7 graphically depicts that dupilumab showed a trend to improvement in sinus total symptom score for CRSsNP (sTSS) only in CRSsNP patients with EOS levels of ≥300 cells/μL and not in patients with EOS levels of <300 cells/μL. sTSS is a composite score including nasal congestion, anterior/posterior rhinorrhea, facial pain/pressure, with a total score 0-9 (a higher score indicates more severe symptoms). The MCID score is −2.

FIG. 8A-FIG. 8D depict that dupilumab resulted in an improvement in sTSS/TSS scores at week 24 in CRSsNP patients with EOS levels of ≥300 cells/μL. (FIG. 8A) Depicts nasal congestion (sTSS), showing a mean difference vs. placebo at week 24 of −0.64 (95% CI: −1.24, −0.03). (FIG. 8B) Depicts facial pain/pressure (sTSS), showing a mean difference vs. placebo at week 24 of −0.24 (95% CI: −0.91, 0.44). (FIG. 8C) Depicts anterior/posterior rhinorrhea (sTSS), showing a mean difference vs. placebo at week 24 of −0.57 (95% CI: −1.20, 0.07). (FIG. 8D) Depicts loss of sense of smell (TSS), showing a mean difference vs. placebo at week 24 of −0.73 (95% CI: −1.35, −0.11).

FIG. 9 graphically depicts that dupilumab led to a trend to clinically meaningful improvement in University of Pennsylvania Smell Identification Test (UPSIT) scores vs. placebo that was observed more prominently in CRSsNP patients with EOS levels of ≥300 cells/μL. The UPSIT score range was 0-40, with a higher score indicating a stronger sense of smell, which is a good outcome. The MCID score is 4.

FIG. 10 graphically depicts that dupilumab improved Sino-Nasal Outcome Test-22 (SNOT-22) score vs. placebo, with a trend to clinically meaningful improvement in CRSsNP patients with EOS levels of ≥300 cells/μL. SNOT-22 is a patient-reported outcome (PRO) that measures health-related quality of life (HRQoL), and 22 items covering symptoms, social/emotional impact, productivity, and sleep over past 2 weeks. Each item is rated on 6-point Likert scale. SNOT-22 used a scale 0-110, with a higher score indicating greater rhinosinusitis-related health burden. The MCID score is −12.

FIG. 11A-FIG. 11C graphically depict that dupilumab decreased biomarkers of Type 2 inflammation. (FIG. 11A) Depicts results for immunoglobulin E (IgE). (FIG. 11B) depicts results for eotaxin-3. (FIG. 11C) Depicts results for thymus and activation regulated chemokine (TARC).

FIG. 12 graphically depicts that dupilumab treatment resulted in a transient increase in mean, but not median, eosinophils followed by return to at or below baseline. No cases of clinically symptomatic eosinophilia were observed.

FIG. 13 graphically depicts the PK profile of CRSsNP patients treated with dupilumab.

FIG. 14 is a tabular safety summary showing the percentages of subjects with CRSsNP treated with dupilumab or placebo having any treatment emergent adverse effects (TEAEs).

### DETAILED DESCRIPTION

It is to be understood that this disclosure is not limited to methods and experimental conditions described, as such methods and conditions may vary. It is also to be understood that the terminology used herein is for the purpose of describing embodiments only, and is not intended to be limiting, because the scope of the disclosure will be limited only by the appended claims.

Unless defined otherwise, all technical and scientific terms used herein have the same meaning as commonly understood by one of ordinary skill in the art to which this disclosure belongs.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

As used herein, the terms “treat,” “treating,” or the like, mean to alleviate symptoms, eliminate the causation of symptoms either on a temporary or permanent basis, or to prevent, reduce, or slow the appearance of symptoms of the named disorder or condition (e.g., to prevent exacerbation of one or more symptoms of CRSsNP).

Although any methods and materials similar or equivalent to those described herein can be used in the practice of the disclosure, the typical methods and materials are now described. All publications mentioned herein are incorporated herein by reference in their entirety.

Methods for Treating Chronic Rhinosinusitis without Nasal Polyps

Described are methods for treating chronic rhinosinusitis without nasal polyps (CRSsNP). As the name suggests, CRSsNP refers to the condition where the sinuses and nasal passages are continuously inflamed but do not produce nasal polyps. CRSsNP is characterized by the presence at least two CRSsNP symptoms for at least 12 weeks. CRSsNP symptoms include, but are not limited to: 1) nasal congestion/obstruction; 2) anterior rhinorrhea (runny nose); 3) posterior rhinorrhea (post nasal drip); 4) loss of sense of smell or taste; 5) facial pain/pressure; and 6) headache. In some embodiments, CRSsNP is driven by type-2 inflammation. In this subset of type-2 CRSsNP patients, transcriptomic and proteomic analyses of surgical sinonasal tissues reveal elevated levels of Type-2 inflammatory markers, such as interleukin (IL)-5, immunoglobulin E (IgE), eosinophilic cationic protein (ECP), Charcot-Leyden crystal galectin (CLC), and the like.

As used herein “uncontrolled CRSsNP” refers to CRSsNP in which the subject has one or more of the following: prior sinonasal surgery for chronic rhinosinusitis; prior systemic corticosteroid (SCS) use for chronic rhinosinusitis; SCS intolerance when previously used for chronic rhinosinusitis; and contraindication of SCS for previous chronic rhinosinusitis treatment. In certain embodiments, SCS was previously administered to the subject at any dose and any duration within two years before treatment with an antibody or antigen-binding fragment thereof described herein.

Asthma or allergic rhinitis are common comorbid conditions for CRSsNP patients. Comorbid asthma and peripheral blood eosinophilia have been associated with a higher risk of need for recurrent sinonasal surgery and need for systemic therapy after sinus surgery. Other clinical features more commonly reported in CRSsNP patients with type-2 endotype include loss of sense of smell/reduced taste; the type-2 CRSsNP patients also have more complaints of headache/migraine as compared with the non-type-2 CRSsNP patients.

The pathological hallmark of CRSsNP-associated upper airway disease is mucus secretion and goblet cell and glandular hyperplasia. Goblet cells are responsible for the production of mucin SAC, one of the main secreted mucins in the human airway. The increase in mucous gland density that occurs in patients with severe CRSsNP contrasts with those with CRSwNP who show a significant decrease in mucous gland density. Glandular hypertrophy and mucous secretion in the airway mucosa are likely to be mediated by various cytokines, including IL-13.

Standard medical therapies, including topical and oral corticosteroids (OCS), and antibiotics, do not provide adequate or lasting control of the symptoms in many patients with CRSsNP. In a study of CRSsNP, 45% of patients “failed” medical therapy, defined as persistent symptoms and 31% remained symptomatic enough to elect to pursue surgery. In the GA²LEN study, 70% to 80% of patients did not have lasting benefits from the medical therapies (including OCS antibiotics, and intranasal corticosteroids [INCS]). Many CRSsNP patients have reported at least one prior surgery or are planning to have surgery soon for better control of symptoms. In patients who have undergone sinonasal surgery, comorbid asthma and peripheral blood eosinophilia are risk factors for recurrence of CRS post-operatively, suggesting that type-2 CRSsNP patients may be more at risk for disease recurrence after surgery. In some embodiments, an IL-4R antagonist described herein is indicated for the treatment of subjects with uncontrolled CRSsNP who have persistent signs and symptoms despite topical or oral corticosteroids or a combination thereof. In some embodiments, an IL-4R antagonist described herein is indicated for the treatment of subjects with type 2 inflammation. In still other embodiments, an IL-4R antagonist described herein is indicated for the treatment of subjects with blood eosinophils count≥300 cells/μL. In some embodiments, asthma, allergic rhinitis, or combination thereof are common comorbid conditions with the type-2 CRSsNP.

A formal diagnosis is made based on sinus computed tomography (CT) scan and/or sinus endoscopy. Based on endoscopic evaluation, CS can be clinically divided as CS with nasal polyps/nasal polyposis (CSwNP) or CS without nasal polyps/nasal polyposis.

As used herein, the term “sinusitis” refers to any inflammatory condition characterized by inflammation of the paranasal sinuses, including inflammation of the maxillary, frontal, ethmoid and/or sphenoid paranasal sinuses.

As used herein, the term “rhinosinusitis” refers to a condition that has symptoms of both rhinitis and sinusitis. Rhinosinusitis includes acute rhinosinusitis and chronic rhinosinusitis. Acute rhinosinusitis can be caused by an infection, such as a bacterial, viral, or fungal infection, or by a chemical irritation. Cigarette-smoke-induced acute rhinosinusitis and chlorine fume-induced chronic rhinosinusitis are examples of acute rhinosinusitis.

The term “rhinitis” refers to an allergic response, such as to a common allergen (“allergic rhinitis,” e.g., perennial allergic rhinitis) or to an environmental irritant (“non-allergic rhinitis”). Symptoms of allergic rhinitis include sneezing; stuffy or runny nose; sinus pressure, and pain or throbbing in the cheeks or nose; and itching in the nose, throat, eyes, and ears.

As used herein, “allergic rhinitis” refers to rhinitis that occurs when the body's immune responds to specific, non-infectious irritants.

Methods for Improving Chronic Rhinosinusitis without Nasal Polyps

Described are methods for improving one or more CRSsNP symptoms in a subject in need thereof, wherein the methods include administering a pharmaceutical composition comprising an interleukin-4 receptor (IL-4R) antagonist to the subject. Some embodiments include a pharmaceutical composition comprising an IL-4R antagonist for use in improving one or more CRSsNP symptoms in a subject in need thereof. For example, an IL-4R receptor antagonist can reduce or improve any one or more of nasal congestion or obstruction, anterior rhinorrhea (runny nose), posterior rhinorrhea (post nasal drip), loss of sense of smell or taste, facial pain or pressure, headaches or any combination thereof.

In some embodiments, administering a pharmaceutical composition comprising an IL-4R antagonist to the subject with CRSsNP improves or reduces the subject's use of topical and systemic or inhaled corticosteroids, need to have endoscopic sinus surgery (ESS) surgery for CRSsNP, sinus opacification as determined by computer tomography (CT) scans, and/or change from baseline in the sinus total symptom score.

The sinus Total Symptom Score (sTSS) is a composite score derived from the following individual items: nasal congestion (NC), anterior/posterior rhinorrhea, and facial pain/pressure. The total score ranges from 0 to 9 and consists of the sum of NC, the averaged rhinorrhea item scores, and facial pain/pressure scores. Higher scores on sTSS indicate greater overall symptom severity. In some embodiments, the CRSsNP sinonasal symptom includes headache. Headache severity is scored on a scale ranging from 0 (“No symptoms”) to 3 (“Severe symptoms—symptoms that are hard to tolerate, cause interference with activities or daily living”). The score is not a part of sTSS.

In some embodiments, administering a pharmaceutical composition comprising an interleukin-4 receptor (IL-4R) antagonist to the subject with CRSsNP improves or reduces “CRSsNP-associated parameters.” Examples of CRSsNP-associated parameters include, but are not limited to: (a) Lund-Mackay (LMK) Score; (b) sinus total symptom score for CRSsNP (sTSS); (c) smell test (University of Pennsylvania Smell Identification Test (UPSIT)) (d) 22-item Sino Nasal Outcome Test (SNOT-22) score; (e) subject-assessed nasal congestion/obstruction, anterior rhinorrhea (runny nose), posterior rhinorrhea (post nasal drip) and loss of sense of smell; (f) number of nocturnal awakenings; (g) Visual Analog Score (VAS) to assess patient-rated rhinosinusitis symptom severity; (h) six-item Asthma Control Questionnaire (ACQ6) score, such as in patients with asthma; (i) Nasal Peak Inspiratory Flow (NPIF); (j) physiological parameters, such as measured by nasal endoscopy and CT scan; and (k) Three Dimensional volumetric measurement of the maxillary sinus.

Lund-Mackay (LMK) Score. The Lund-Mackay scoring system is based on localization with points given for degree of opacification: 0=normal, 1=partial opacification, 2=total opacification. These points are then applied to the maxillary, anterior ethmoid, posterior ethmoid, sphenoid, and frontal sinus on each side. The osteomeatal complex is graded as 0=not occluded, or 2=occluded deriving a maximum score of 12 per side. For patients in whom the osteomeatal complex (OC) is missing (because of a previous surgery) the location of the former OC is considered, and a score is provided, as if the OC was there. LMK uses a scale of 0-24, wherein a higher score is worse and radiographically more opacified. The minimally clinical important differences (MCID) score for LMK is −4.

Administration of an IL-4R antagonist to a subject in need thereof causes a decrease in LMK score from baseline by about 1 point, about 2 points, about 3 points, about 4 points, about 5 points, about 6 points, about 7 points, about 8 points, about 9 points, about 10 points, about 11 points, about 12 points, about 13 points, about 14 points, about 15 points, about 16 points, about 17 points, about 18 points, about 19 points, about 20 points, about 21 points, about 22 points, about 23 points, or about 24 points at week 4, week 6, week 12, week 16, week 24, or week 52. Thus, in one example, administration of an IL-4R antagonist to a subject in need thereof causes a decrease in LMK score from baseline by about 1 point, about 2 points, about 3 points, about 4 points, about 5 points, about 6 points, about 7 points, about 8 points, about 9 points, about 10 points, about 11 points, about 12 points, about 13 points, about 14 points, about 15 points, about 16 points, about 17 points, about 18 points, about 19 points, about 20 points, about 21 points, about 22 points, about 23 points, or about 24 points at week 4. In a further example, administration of an IL-4R antagonist to a subject in need thereof causes a decrease in LMK score from baseline by about 1 point, about 2 points, about 3 points, about 4 points, about 5 points, about 6 points, about 7 points, about 8 points, about 9 points, about 10 points, about 11 points, about 12 points, about 13 points, about 14 points, about 15 points, about 16 points, about 17 points, about 18 points, about 19 points, about 20 points, about 21 points, about 22 points, about 23 points, or about 24 points at week 6. In a further example, administration of an IL-4R antagonist to a subject in need thereof causes a decrease in LMK score from baseline by about 1 point, about 2 points, about 3 points, about 4 points, about 5 points, about 6 points, about 7 points, about 8 points, about 9 points, about 10 points, about 11 points, about 12 points, about 13 points, about 14 points, about 15 points, about 16 points, about 17 points, about 18 points, about 19 points, about 20 points, about 21 points, about 22 points, about 23 points, or about 24 points at week 12. In a further example, administration of an IL-4R antagonist to a subject in need thereof causes a decrease in LMK from baseline by about 1 point, about 2 points, about 3 points, about 4 points, about 5 points, about 6 points, about 7 points, about 8 points, about 9 points, about 10 points, about 11 points, about 12 points, about 13 points, about 14 points, about 15 points, about 16 points, about 17 points, about 18 points, about 19 points, about 20 points, about 21 points, about 22 points, about 23 points, or about 24 points at week 24. In a further example, administration of an IL-4R antagonist to a subject in need thereof causes a decrease in LMK score from baseline by about 1 point, about 2 points, about 3 points, about 4 points, about 5 points, about 6 points, about 7 points, about 8 points, about 9 points, about 10 points, about 11 points, about 12 points, about 13 points, about 14 points, about 15 points, about 16 points, about 17 points, about 18 points, about 19 points, about 20 points, about 21 points, about 22 points, about 23 points, or about 24 points at week 52. The decrease in LMK score can be detected as early as week 4, and as late as week 52 following administration of the IL-4R antagonist.

Sinus Total Symptom Score for CRSsNP (sTSS). sTSS includes facial pain/pressure instead of sense of smell, which is included in TSS, and differentiation is made based on the clinical picture of CRSwNP (TSS) vs. CRSsNP (sTSS). sTSS is a composite score assessing nasal congestion, anterior/posterior rhinorrhea, and facial pain/pressure that uses a scale of 0-9, in which a higher score is worse, and clinically more symptomatic. The MCID score for sTSS is −2.

Administration of an IL-4R antagonist to a subject in need thereof causes a decrease in sTSS score from baseline by about 1 point, about 2 points, about 3 points, about 4 points, about 5 points, about 6 points, about 7 points, about 8 points, or about 9 points at week 4, week 6, week 12, week 16, week 24, or week 52. Thus, in one example, administration of an IL-4R antagonist to a subject in need thereof causes a decrease in sTSS score from baseline by about 1 point, about 2 points, about 3 points, about 4 points, about 5 points, about 6 points, about 7 points, about 8 points, or about 9 points at week 4. In a further example, administration of an IL-4R antagonist to a subject in need thereof causes a decrease in sTSS score from baseline by about 1 point, about 2 points, about 3 points, about 4 points, about 5 points, about 6 points, about 7 points, about 8 points, or about 9 points at week 6. In a further example, administration of an IL-4R antagonist to a subject in need thereof causes a decrease in sTSS score from baseline by about 1 point, about 2 points, about 3 points, about 4 points, about 5 points, about 6 points, about 7 points, about 8 points, or about 9 points at week 12. In a further example, administration of an IL-4R antagonist to a subject in need thereof causes a decrease in sTSS score from baseline by about 1 point, about 2 points, about 3 points, about 4 points, about 5 points, about 6 points, about 7 points, about 8 points, or about 9 points at week 24. In a further example, administration of an IL-4R antagonist to a subject in need thereof causes a decrease in sTSS score from baseline by about 1 point, about 2 points, about 3 points, about 4 points, about 5 points, about 6 points, about 7 points, about 8 points, or about 9 points at week 52. The decrease in sTSS score can be detected as early as week 4, and as late as week 52 following administration of the IL-4R antagonist.

University of Pennsylvania Smell Identification Test (UPSIT). The UPSIT is a method to quantitatively assess human olfactory function. The test consists of samples of odorants, and the subject must describe the odor. The score is based on the number of correct answers. This test can distinguish patients with a normal sense of smell (“normosmia”) from those with different levels of reduction (“mild, moderate and severe microsmia”) or loss (“anosmia”). UPSIT assesses 40 odorants with four multiple choice answers to assess sense of smell that uses a scale of 0-40, wherein a higher score is better and indicates stronger olfactory function. The MCID score for UPSIT is 2 (Mahadev A, Kallogjeri D, Piccirillo J F. Validation of Minimal Clinically Important Difference (MCID) for University of Pennsylvania Smell Identification Test (UPSIT). Am J Rhinol Allergy. 2024 March; 38(2):123-132. doi: 10.1177/19458924231218037. Epub 2023 Dec. 6. PMID: 38055971).

Administration of an IL-4R antagonist to a subject in need thereof causes an increase in UPSIT score from baseline by about 1 point, about 2 points, about 3 points, about 4 points, about 5 points, about 6 points, about 7 points, about 8 points, about 9 points, about 10 points, about 11 points, about 12 points, about 13 points, about 14 points, about 15 points, about 16 points, about 17 points, about 18 points, about 19 points, about 20 points, about 21 points, about 22 points, about 23 points, about 24 points, about 25 points, about 26 points, about 27 points, about 28 points, about 29 points, about 30 points, about 31 points, about 32 points, about 33 points, about 34 points, about 35 points, about 36 points, about 37 points, about 38 points, about 39 points, or about 40 points at week 4, week 6, week 12, week 18, week 24 week 36, or week 52. Thus, in one example, administration of an IL-4R antagonist to a subject in need thereof causes an increase in UPSIT score from baseline by about 1 point, about 2 points, about 3 points, about 4 points, about 5 points, about 6 points, about 7 points, about 8 points, about 9 points, about 10 points, about 11 points, about 12 points, about 13 points, about 14 points, about 15 points, about 16 points, about 17 points, about 18 points, about 19 points, about 20 points, about 21 points, about 22 points, about 23 points, about 24 points, about 25 points, about 26 points, about 27 points, about 28 points, about 29 points, about 30 points, about 31 points, about 32 points, about 33 points, about 34 points, about 35 points, about 36 points, about 37 points, about 38 points, about 39 points, or about 40 points at week 4. In a further example, administration of an IL-4R antagonist to a subject in need thereof causes an increase in UPSIT score from baseline by about 1 point, about 2 points, about 3 points, about 4 points, about 5 points, about 6 points, about 7 points, about 8 points, about 9 points, about 10 points, about 11 points, about 12 points, about 13 points, about 14 points, about 15 points, about 16 points, about 17 points, about 18 points, about 19 points, about 20 points, about 21 points, about 22 points, about 23 points, about 24 points, about 25 points, about 26 points, about 27 points, about 28 points, about 29 points, about 30 points, about 31 points, about 32 points, about 33 points, about 34 points, about 35 points, about 36 points, about 37 points, about 38 points, about 39 points, or about 40 points at week 6. In a further example, administration of an IL-4R antagonist to a subject in need thereof causes an increase in UPSIT score from baseline by about 1 point, about 2 points, about 3 points, about 4 points, about 5 points, about 6 points, about 7 points, about 8 points, about 9 points, about 10 points, about 11 points, about 12 points, about 13 points, about 14 points, about 15 points, about 16 points, about 17 points, about 18 points, about 19 points, about 20 points, about 21 points, about 22 points, about 23 points, about 24 points, about 25 points, about 26 points, about 27 points, about 28 points, about 29 points, about 30 points, about 31 points, about 32 points, about 33 points, about 34 points, about 35 points, about 36 points, about 37 points, about 38 points, about 39 points, or about 40 points at week 12. In a further example, administration of an IL-4R antagonist to a subject in need thereof causes an increase in UPSIT score from baseline by about 1 point, about 2 points, about 3 points, about 4 points, about 5 points, about 6 points, about 7 points, about 8 points, about 9 points, about 10 points, about 11 points, about 12 points, about 13 points, about 14 points, about 15 points, about 16 points, about 17 points, about 18 points, about 19 points, about 20 points, about 21 points, about 22 points, about 23 points, about 24 points, about 25 points, about 26 points, about 27 points, about 28 points, about 29 points, about 30 points, about 31 points, about 32 points, about 33 points, about 34 points, about 35 points, about 36 points, about 37 points, about 38 points, about 39 points, or about 40 points at week 24. In a further example, administration of an IL-4R antagonist to a subject in need thereof causes an increase in UPSIT score from baseline by about 1 point, about 2 points, about 3 points, about 4 points, about 5 points, about 6 points, about 7 points, about 8 points, about 9 points, about 10 points, about 11 points, about 12 points, about 13 points, about 14 points, about 15 points, about 16 points, about 17 points, about 18 points, about 19 points, about 20 points, about 21 points, about 22 points, about 23 points, about 24 points, about 25 points, about 26 points, about 27 points, about 28 points, about 29 points, about 30 points, about 31 points, about 32 points, about 33 points, about 34 points, about 35 points, about 36 points, about 37 points, about 38 points, about 39 points, or about 40 points at week 52. The increase in UPSIT score can be detected as early as week 4, and as late as week 24 or later following administration of the IL-4R antagonist.

22-Item Sinonasal Outcome Test (SNOT-22) Score. According to certain embodiments, administration of an IL-4R antagonist to a patient result in a decrease from baseline of 22-item Sinonasal Outcome Test (SNOT-22). The SNOT-22 is a questionnaire to assess the impact of chronic rhinosinusitis (CRS) on health related quality of life (HRQoL). The questionnaire measures items related to sinonasal conditions and surgical treatments. The score ranges from 0 to 110, and higher scores imply greater impact of CRS on HRQoL (Hopkins et al 2009, Clin. Otolaryngol. 34: 447-454). The MCID score for UPSIT is −12.

In some embodiments, the therapeutic methods result in a decrease in SNOT-22 score from baseline of at least 12 points at week 4 to week 52 following administration of the IL-4R antagonist. In some embodiments, the present disclosure includes an IL-4R antagonist for use in decreasing a SNOT-22 score from baseline of at least 12 points at week 4 to week 52. For example, administration of an IL-4R antagonist will result in a decrease in SNOT-22 score at week 4, week 6, week 8, week 12, or week 16, week 24, week 36, and week 52 following initiation of treatment. In some embodiments, administration of an IL-4R antagonist to a subject in need thereof causes a decrease in SNOT-22 score from baseline by about 10 points, about 15 points, about 20 points, about 25 points, about 30 points, about 35 points, about 40 points, about 45 points, about 50 points, about 55 points, about 60 points, about 65 points, about 70 points, about 75 points, about 80 points, about 85 points, about 90 points, about 95 points, about 100 points, about 105 points, or about 110 points at week 4, week 6, week 8, week 12, week 16, week 24, week 36, or week 52. Thus, in one embodiment, administration of an IL-4R antagonist to a subject in need thereof causes a decrease in SNOT-22 score from baseline by about 10 points, about 15 points, about 20 points, about 25 points, about 30 points, about 35 points, about 40 points, about 45 points, about 50 points, about 55 points, about 60 points, about 65 points, about 70 points, about 75 points, about 80 points, about 85 points, about 90 points, about 95 points, about 100 points, about 105 points, or about 110 points at week 4. In another embodiment, administration of an IL-4R antagonist to a subject in need thereof causes a decrease in SNOT-22 score from baseline by about 10 points, about 15 points, about 20 points, about 25 points, about 30 points, about 35 points, about 40 points, about 45 points, about 50 points, about 55 points, about 60 points, about 65 points, about 70 points, about 75 points, about 80 points, about 85 points, about 90 points, about 95 points, about 100 points, about 105 points, or about 110 points at week 6. In a further embodiment, administration of an IL-4R antagonist to a subject in need thereof causes a decrease in SNOT-22 score from baseline by about 10 points, about 15 points, about 20 points, about 25 points, about 30 points, about 35 points, about 40 points, about 45 points, about 50 points, about 55 points, about 60 points, about 65 points, about 70 points, about 75 points, about 80 points, about 85 points, about 90 points, about 95 points, about 100 points, about 105 points, or about 110 points at week 8. In yet another embodiment, administration of an IL-4R antagonist to a subject in need thereof causes a decrease in SNOT-22 score from baseline by about 10 points, about 15 points, about 20 points, about 25 points, about 30 points, about 35 points, about 40 points, about 45 points, about 50 points, about 55 points, about 60 points, about 65 points, about 70 points, about 75 points, about 80 points, about 85 points, about 90 points, about 95 points, about 100 points, about 105 points, or about 110 points at week 12. In yet another embodiment, administration of an IL-4R antagonist to a subject in need thereof causes a decrease in SNOT-22 score from baseline by about 10 points, about 15 points, about 20 points, about 25 points, about 30 points, about 35 points, about 40 points, about 45 points, about 50 points, about 55 points, about 60 points, about 65 points, about 70 points, about 75 points, about 80 points, about 85 points, about 90 points, about 95 points, about 100 points, about 105 points, or about 110 points at week 24. In yet another embodiment, administration of an IL-4R antagonist to a subject in need thereof causes a decrease in SNOT-22 score from baseline by about 10 points, about 15 points, about 20 points, about 25 points, about 30 points, about 35 points, about 40 points, about 45 points, about 50 points, about 55 points, about 60 points, about 65 points, about 70 points, about 75 points, about 80 points, about 85 points, about 90 points, about 95 points, about 100 points, about 105 points, or about 110 points at week 36. In still another embodiment, administration of an IL-4R antagonist to a subject in need thereof causes a decrease in SNOT-22 score from baseline by about 10 points, about 15 points, about 20 points, about 25 points, about 30 points, about 35 points, about 40 points, about 45 points, about 50 points, about 55 points, about 60 points, about 65 points, about 70 points, about 75 points, about 80 points, about 85 points, about 90 points, about 95 points, about 100 points, about 105 points, or about 110 points at week 52.

Individual and Total Nasal Symptom Score. Subject-assessed symptoms are assayed by responding to morning and evening individual rhinosinusitis symptom questions using a 0-3 categorical scale (where 0=no symptoms, 1=mild symptoms, 2=moderate symptoms and 3=severe symptoms), and including the symptoms of congestion and/or obstruction, anterior rhinorrhea, posterior rhinorrhea, and loss of sense of smell. Nasal symptoms can be assayed in the day (AM), at night (PM) or both AM and PM.

A loss of sense of smell can be tracked. Administration of an IL-4R antagonist can result, for example, in a decrease in loss of sense of smell (viz., achieving a lower number on the scale) from baseline compared to week 4 to week 16 following initiation of treatment with a pharmaceutical composition comprising an anti-IL-4R antagonist. For example, a decrease in loss of sense of smell from baseline (e.g., from about 0.5, about 1.0, about 1.5, about 2.0, about 2.5 or about 3.0) can be detected at week 4, week 6, week 8, week 12, week 16, week 24, week 36, or week 52 following initiation of treatment. Thus, in an example, a decrease in loss of sense of smell from baseline (e.g., from about 0.5, about 1.0, about 1.5, about 2.0, about 2.5 or about 3.0) can be detected at week 4 following initiation of treatment. In a further example, a decrease in loss of sense of smell from baseline (e.g., from about 0.5, about 1.0, about 1.5, about 2.0, about 2.5 or about 3.0) can be detected at week 6 following initiation of treatment. In a further example, a decrease in loss of sense of smell from baseline (e.g., from about 0.5, about 1.0, about 1.5, about 2.0, about 2.5 or about 3.0) can be detected at week 8 following initiation of treatment. In a further example, a decrease in loss of sense of smell from baseline (e.g., from about 0.5, about 1.0, about 1.5, about 2.0, about 2.5 or about 3.0) can be detected at week 12 following initiation of treatment. In a further example, a decrease in loss of sense of smell from baseline (e.g., from about 0.5, about 1.0, about 1.5, about 2.0, about 2.5 or about 3.0) can be detected at week 16 following initiation of treatment. Administration of an IL-4R antagonist to a subject in need thereof can cause a decrease in loss of sense of smell symptom score from baseline by about 0.5, about 1.0, about 1.5, about 2.0, about 2.5 or about 3.0 or more at week 4, week 8, week 12, week 16, week 24, or week 52, for example. Administration of an IL-4R antagonist to a subject in need thereof can cause a decrease in loss of sense of smell symptom score from baseline by about 0.5, about 1.0, about 1.5, about 2.0, about 2.5 or about 3.0 or more at week 52.

A decrease in congestion and/or obstruction can be tracked. Administration of an IL-4R antagonist can result, for example, in a decrease in congestion and/or obstruction (viz., achieving a lower number on the scale) from baseline compared to week 4 to week 16 following initiation of treatment with a pharmaceutical composition comprising an anti-IL-4R antagonist. For example, a decrease in congestion and/or obstruction from baseline (e.g., from about 0.5, about 1.0, about 1.5, about 2.0, about 2.5 or about 3.0) can be detected at week 4, week 6, week 8, week 12, or week 16 following initiation of treatment. Thus, in an example, a decrease in congestion and/or obstruction from baseline (e.g., from about 0.5, about 1.0, about 1.5, about 2.0, about 2.5 or about 3.0) can be detected at week 4 following initiation of treatment. In a further example, a decrease in congestion and/or obstruction from baseline (e.g., from about 0.5, about 1.0, about 1.5, about 2.0, about 2.5 or about 3.0) can be detected at week 6 following initiation of treatment. In a further example, a decrease in congestion and/or obstruction from baseline (e.g., from about 0.5, about 1.0, about 1.5, about 2.0, about 2.5 or about 3.0) can be detected at week 8 following initiation of treatment. In a further example, a decrease in congestion and/or obstruction from baseline (e.g., from about 0.5, about 1.0, about 1.5, about 2.0, about 2.5 or about 3.0) can be detected at week 12 following initiation of treatment. In a further example, a decrease in congestion and/or obstruction from baseline (e.g., from about 0.5, about 1.0, about 1.5, about 2.0, about 2.5 or about 3.0) can be detected at week 16 following initiation of treatment. Administration of an IL-4R antagonist to a subject in need thereof can cause a decrease in congestion and/or obstruction symptom score from baseline by about 0.5, about 1.0, about 1.5, about 2.0, about 2.5 or about 3.0 or more at week 4, week 8, week 12, or week 16, for example.

A decrease in runny nose can be tracked. Administration of an IL-4R antagonist can result, for example, in a decrease in runny nose (viz., achieving a lower number on the scale) from baseline compared to week 4 to week 16 following initiation of treatment with a pharmaceutical composition comprising an anti-IL-4R antagonist. For example, a decrease in runny nose from baseline (e.g., from about 0.5, about 1.0, about 1.5, about 2.0, about 2.5 or about 3.0) can be detected at week 4, week 6, week 8, week 12, or week 16 following initiation of treatment. Thus, in an example, a decrease in runny nose from baseline (e.g., from about 0.5, about 1.0, about 1.5, about 2.0, about 2.5 or about 3.0) can be detected at week 4 following initiation of treatment. In a further example, a decrease in runny nose from baseline (e.g., from about 0.5, about 1.0, about 1.5, about 2.0, about 2.5 or about 3.0) can be detected at week 6 following initiation of treatment. In a further example, a decrease in runny nose from baseline (e.g., from about 0.5, about 1.0, about 1.5, about 2.0, about 2.5 or about 3.0) can be detected at week 8 following initiation of treatment. In a further example, a decrease in runny nose from baseline (e.g., from about 0.5, about 1.0, about 1.5, about 2.0, about 2.5 or about 3.0) can be detected at week 12 following initiation of treatment. In a further example, a decrease in runny nose from baseline (e.g., from about 0.5, about 1.0, about 1.5, about 2.0, about 2.5 or about 3.0) can be detected at week 16 following initiation of treatment. Administration of an IL-4R antagonist to a subject in need thereof can cause a decrease in runny nose symptom score from baseline by about 0.5, about 1.0, about 1.5, about 2.0, about 2.5 or about 3.0 or more at week 4, week 8, week 12, or week 16, for example.

A decrease in post nasal drip can be tracked. Administration of an IL-4R antagonist can result, for example, in a decrease in runny nose (viz., achieving a lower number on the scale) from baseline compared to week 4 to week 16 following initiation of treatment with a pharmaceutical composition comprising an anti-IL-4R antagonist. For example, a decrease in post nasal drip from baseline (e.g., from about 0.5, about 1.0, about 1.5, about 2.0, about 2.5 or about 3.0) can be detected at week 4, week 6, week 8, week 12, or week 16 following initiation of treatment. Thus, in an example, a decrease in post nasal drip from baseline (e.g., from about 0.5, about 1.0, about 1.5, about 2.0, about 2.5 or about 3.0) can be detected at week 4 following initiation of treatment. In a further example, a decrease in post nasal drip from baseline (e.g., from about 0.5, about 1.0, about 1.5, about 2.0, about 2.5 or about 3.0) can be detected at week 6 following initiation of treatment. In a further example, a decrease in post nasal drip from baseline (e.g., from about 0.5, about 1.0, about 1.5, about 2.0, about 2.5 or about 3.0) can be detected at week 8 following initiation of treatment. In a further example, a decrease in post nasal drip from baseline (e.g., from about 0.5, about 1.0, about 1.5, about 2.0, about 2.5 or about 3.0) can be detected at week 12 following initiation of treatment. In a further example, a decrease in post nasal drip from baseline (e.g., from about 0.5, about 1.0, about 1.5, about 2.0, about 2.5 or about 3.0) can be detected at week 16 following initiation of treatment. Administration of an IL-4R antagonist to a subject in need thereof can cause a decrease in post nasal drip symptom score from baseline by about 0.5, about 1.0, about 1.5, about 2.0, about 2.5 or about 3.0 or more at week 4, week 8, week 12, or week 16, for example.

A measure of night-time awakenings can also be tracked. For example, a measure of night-time awakenings can be assessed according to the following scores based on subject self-assessment: 0=no symptoms, slept through the night; 1=slept well, but some complaints in the morning; 2=woke up once because of rhinosinusitis symptoms (including early awakening); 3=woke up several times because of symptoms (including early awakening); 4=bad night, awake most of the night because of symptoms. Administration of an IL-4R antagonist can result, for example, in a decrease in average number of nighttime awakenings per night from baseline of at least about 0.10 times per night at week 4 to week 16 following initiation of treatment with a pharmaceutical composition comprising an anti-IL-4R antagonist. For example, a decrease in frequency of nighttime awakenings per night from baseline of at least about 0.10 times per night can be detected at week 4, week 6, week 8, week 12, or week 16 following initiation of treatment. Administration of an IL-4R antagonist to a subject in need thereof can cause a decrease in average number of nighttime awakenings per night from baseline by about 0.10 times per night, 0.15 times per night, 0.20 times per night, 0.25 times per night, 0.30 times per night, 0.35 times per night, 0.40 times per night, 0.45 times per night, 0.50 times per night, 0.55 times per night, 0.60 times per night, 0.65 times per night, 0.70 times per night, 0.75 times per night, 0.80 times per night, 0.85 times per night, 0.90 times per night, 0.95 times per night, 1.0 time per night, 2.0 times per night, or more at week 4, week 8, week 12, or week 16, for example. Thus, in one example, administration of an IL-4R antagonist to a subject in need thereof can cause a decrease in average number of nighttime awakenings per night from baseline by about 0.10 times per night, 0.15 times per night, 0.20 times per night, 0.25 times per night, 0.30 times per night, 0.35 times per night, 0.40 times per night, 0.45 times per night, 0.50 times per night, 0.55 times per night, 0.60 times per night, 0.65 times per night, 0.70 times per night, 0.75 times per night, 0.80 times per night, 0.85 times per night, 0.90 times per night, 0.95 times per night, 1.0 time per night, 2.0 times per night, or more at week 4. In a further example, administration of an IL-4R antagonist to a subject in need thereof can cause a decrease in average number of nighttime awakenings per night from baseline by about 0.10 times per night, 0.15 times per night, 0.20 times per night, 0.25 times per night, 0.30 times per night, 0.35 times per night, 0.40 times per night, 0.45 times per night, 0.50 times per night, 0.55 times per night, 0.60 times per night, 0.65 times per night, 0.70 times per night, 0.75 times per night, 0.80 times per night, 0.85 times per night, 0.90 times per night, 0.95 times per night, 1.0 time per night, 2.0 times per night, or more at week 8. In a further example, administration of an IL-4R antagonist to a subject in need thereof can cause a decrease in average number of nighttime awakenings per night from baseline by about 0.10 times per night, 0.15 times per night, 0.20 times per night, 0.25 times per night, 0.30 times per night, 0.35 times per night, 0.40 times per night, 0.45 times per night, 0.50 times per night, 0.55 times per night, 0.60 times per night, 0.65 times per night, 0.70 times per night, 0.75 times per night, 0.80 times per night, 0.85 times per night, 0.90 times per night, 0.95 times per night, 1.0 time per night, 2.0 times per night, or more at week 12. In a further example, administration of an IL-4R antagonist to a subject in need thereof can cause a decrease in average number of nighttime awakenings per night from baseline by about 0.10 times per night, 0.15 times per night, 0.20 times per night, 0.25 times per night, 0.30 times per night, 0.35 times per night, 0.40 times per night, 0.45 times per night, 0.50 times per night, 0.55 times per night, 0.60 times per night, 0.65 times per night, 0.70 times per night, 0.75 times per night, 0.80 times per night, 0.85 times per night, 0.90 times per night, 0.95 times per night, 1.0 time per night, 2.0 times per night, or more at week 16. In a further example, administration of an IL-4R antagonist to a subject in need thereof can cause a decrease in average number of nighttime awakenings per night from baseline by about 0.10 times per night, 0.15 times per night, 0.20 times per night, 0.25 times per night, 0.30 times per night, 0.35 times per night, 0.40 times per night, 0.45 times per night, 0.50 times per night, 0.55 times per night, 0.60 times per night, 0.65 times per night, 0.70 times per night, 0.75 times per night, 0.80 times per night, 0.85 times per night, 0.90 times per night, 0.95 times per night, 1.0 time per night, 2.0 times per night, or more at week 24. In a further example, administration of an IL-4R antagonist to a subject in need thereof can cause a decrease in average number of nighttime awakenings per night from baseline by about 0.10 times per night, 0.15 times per night, 0.20 times per night, 0.25 times per night, 0.30 times per night, 0.35 times per night, 0.40 times per night, 0.45 times per night, 0.50 times per night, 0.55 times per night, 0.60 times per night, 0.65 times per night, 0.70 times per night, 0.75 times per night, 0.80 times per night, 0.85 times per night, 0.90 times per night, 0.95 times per night, 1.0 time per night, 2.0 times per night, or more at week 52.

Visual Analog Score (VAS). The VAS is a measure to assess patient-related rhinosinusitis symptom severity on a scale of 1 to 10. Mild symptoms are indicated by a score of 0 to 3, moderate symptoms are indicated by a VAS score of >3 to 7, and severe symptoms are indicated by a VAS score of >7 to 10. Administration of an IL-4R antagonist to a subject in need thereof causes a decrease in VAS score from baseline of about 0.5 point, 1 point, 1.5 points, 2 points, 2.5 points, 3 points, 3.5 points, 4 points, or more at week 4, week 6, week 12, week 24 or week 52. Thus, in one example, administration of an IL-4R antagonist to a subject in need thereof causes a decrease in VAS score from baseline of about 0.5 point, 1 point, 1.5 points, 2 points, 2.5 points, 3 points, 3.5 points, 4 points, or more at week 4. In a further example, administration of an IL-4R antagonist to a subject in need thereof causes a decrease in VAS score from baseline of about 0.5 point, 1 point, 1.5 points, 2 points, 2.5 points, 3 points, 3.5 points, 4 points, or more at week 6. In a further example, administration of an IL-4R antagonist to a subject in need thereof causes a decrease in VAS score from baseline of about 0.5 point, 1 point, 1.5 points, 2 points, 2.5 points, 3 points, 3.5 points, 4 points, or more at week 12. The decrease in VAS score can be detected as early as week 4, and as late as week 52 following administration of the IL-4R antagonist.

6-Item Asthma Control Questionnaire (ACQ) Score. The ACQ6 measures both the adequacy of asthma control and change in asthma control, that occurs either spontaneously or as a result of treatment. The ACQ-6 has 6 questions that assess the most common asthma symptoms: 1) frequency in past week awoken by asthma during the night; 2) severity of asthma symptoms in the morning; 3) limitation of daily activities due to asthma; 4) shortness of breath due to asthma; 5) frequency of wheezing; and 6) short-acting bronchodilator use. The six questions on the ACQ6 reflect the top-scoring six asthma symptoms: woken at night by symptoms, wake in the mornings with symptoms, limitation of daily activities, shortness of breath and wheeze. Patients respond to the symptom questions on a 7-point scale (0=no impairment, totally controlled; 6=maximum impairment, severely uncontrolled). A higher score indicates lower asthma control.

In some embodiments therapeutic methods result in a decrease in ACQ6 score from baseline of at least 0.10 point at week 24 following initiation of treatment with a pharmaceutical composition comprising an anti-IL-4R antagonist. For example, according to the present disclosure, administration of an IL-4R antagonist to a subject in need thereof causes a decrease in ACQ score from baseline of about 0.10 points, 0.15 points, 0.20 points, 0.25 points, 0.30 points, 0.35 points, 0.40 points, 0.45 points, 0.50 points, 0.55 points, 0.60 points, 0.65 points, 0.70 points, 0.75 points, 0.80 points, 0.85 points, or more at week 4, week 6, week 12, week 18, week 24, or week 52 Thus, in one example, administration of an IL-4R antagonist to a subject in need thereof causes a decrease in ACQ score from baseline of about 0.10 points, 0.15 points, 0.20 points, 0.25 points, 0.30 points, 0.35 points, 0.40 points, 0.45 points, 0.50 points, 0.55 points, 0.60 points, 0.65 points, 0.70 points, 0.75 points, 0.80 points, 0.85 points, or more at week 4. In a further example, administration of an IL-4R antagonist to a subject in need thereof causes a decrease in ACQ score from baseline of about 0.10 points, 0.15 points, 0.20 points, 0.25 points, 0.30 points, 0.35 points, 0.40 points, 0.45 points, 0.50 points, 0.55 points, 0.60 points, 0.65 points, 0.70 points, 0.75 points, 0.80 points, 0.85 points, or more at week 6. In a further example, administration of an IL-4R antagonist to a subject in need thereof causes a decrease in ACQ score from baseline of about 0.10 points, 0.15 points, 0.20 points, 0.25 points, 0.30 points, 0.35 points, 0.40 points, 0.45 points, 0.50 points, 0.55 points, 0.60 points, 0.65 points, 0.70 points, 0.75 points, 0.80 points, 0.85 points, or more at week 12. The decrease in ACQ score can be detected as early as week 4, and as late as week 52 following administration of the IL-4R antagonist.

Nasal Peak Inspiratory Flow (NPIF). The Nasal Peak Inspiratory Flow (NPIF) represents a physiologic measure of air flow through both nasal cavities during forced inspiration and/or expiration expressed in liters per minute. Nasal inspiration correlates most with the subjective feeling of obstruction and is used to monitor nasal flow. Administration of an IL-4R antagonist to a subject in need thereof causes an increase in NPIF from baseline by about 0.10 liters per minute, 0.15 liters per minute, 0.20 liters per minute, 0.25 liters per minute, 0.30 liters per minute, 0.35 liters per minute, 0.40 liters per minute, 0.45 liters per minute, 0.50 liters per minute, 0.55 liters per minute, 0.60 liters per minute, 0.65 liters per minute, 0.70 liters per minute, 0.75 liters per minute, 0.80 liters per minute, 0.85 liters per minute, or more at week 4, week 6 or week 12. Thus, in one example, administration of an IL-4R antagonist to a subject in need thereof causes an increase in NPIF from baseline by about 0.10 liters per minute, 0.15 liters per minute, 0.20 liters per minute, 0.25 liters per minute, 0.30 liters per minute, 0.35 liters per minute, 0.40 liters per minute, 0.45 liters per minute, 0.50 liters per minute, 0.55 liters per minute, 0.60 liters per minute, 0.65 liters per minute, 0.70 liters per minute, 0.75 liters per minute, 0.80 liters per minute, 0.85 liters per minute, or more at week 4. In a further example, administration of an IL-4R antagonist to a subject in need thereof causes an increase in NPIF from baseline by about 0.10 liters per minute, 0.15 liters per minute, 0.20 liters per minute, 0.25 liters per minute, 0.30 liters per minute, 0.35 liters per minute, 0.40 liters per minute, 0.45 liters per minute, 0.50 liters per minute, 0.55 liters per minute, 0.60 liters per minute, 0.65 liters per minute, 0.70 liters per minute, 0.75 liters per minute, 0.80 liters per minute, 0.85 liters per minute, or more at week 6. In a further example, administration of an IL-4R antagonist to a subject in need thereof causes an increase in NPIF from baseline by about 0.10 liters per minute, 0.15 liters per minute, 0.20 liters per minute, 0.25 liters per minute, 0.30 liters per minute, 0.35 liters per minute, 0.40 liters per minute, 0.45 liters per minute, 0.50 liters per minute, 0.55 liters per minute, 0.60 liters per minute, 0.65 liters per minute, 0.70 liters per minute, 0.75 liters per minute, 0.80 liters per minute, 0.85 liters per minute, or more at week 12. In a further example, administration of an IL-4R antagonist to a subject in need thereof causes an increase in NPIF from baseline by about 0.10 liters per minute, 0.15 liters per minute, 0.20 liters per minute, 0.25 liters per minute, 0.30 liters per minute, 0.35 liters per minute, 0.40 liters per minute, 0.45 liters per minute, 0.50 liters per minute, 0.55 liters per minute, 0.60 liters per minute, 0.65 liters per minute, 0.70 liters per minute, 0.75 liters per minute, 0.80 liters per minute, 0.85 liters per minute, or more at week 24. In a further example, administration of an IL-4R antagonist to a subject in need thereof causes an increase in NPIF from baseline by about 0.10 liters per minute, 0.15 liters per minute, 0.20 liters per minute, 0.25 liters per minute, 0.30 liters per minute, 0.35 liters per minute, 0.40 liters per minute, 0.45 liters per minute, 0.50 liters per minute, 0.55 liters per minute, 0.60 liters per minute, 0.65 liters per minute, 0.70 liters per minute, 0.75 liters per minute, 0.80 liters per minute, 0.85 liters per minute, or more at week 52. The increase in NPIF score can be detected as early as week 4, and as late as week 52 following administration of the IL-4R antagonist.

Physiological parameters. Efficacy of an IL-4R antagonist can be assayed by measuring the effect of physiological parameters, such as within the nasal cavities, such as by nasal endoscopy or computed tomography (CT) scan. Sinonasal surgery can include endoscopic sinus surgery (ESS). ESS includes all the current procedural terminology (CPT) codes used for sinus surgery, which implies restitution of physiology and is used to create a sinus cavity opening that incorporates the natural ostium; allows adequate sinus ventilation; facilitates mucociliary clearance; facilitates instillation of topical therapies: ESS may include also balloon sinuplasty: endoscopic nasal surgery that uses small balloon catheters that inflate to drain the large nasal sinuses. Or “Full ESS’ is defined as complete sinus opening including anterior and posterior ethmoidectomy, maxillary antrostomy (could be large), sphenoidotomy and frontal sinusotomy in the same context as ‘full’ (e.g., Draf III) but could also include extension beyond the confines of sinuses i.e., skull base, orbit, pterygopalatine and infratemporal fossa, or radical functional endoscopic sinus surgery which includes significant removal of inflamed/dysfunctional mucosa.

Three-Dimensional volumetric measurement of maxillary sinus. This value is used to calculate the volume of air (mL); the volume of mucosa (mL); the percent sinus occupied by disease; and the thickness of lateral wall in the maxillary sinus. Administration of an IL-4R antagonist to a subject in need thereof causes an increase in the Three-Dimensional volumetric measurement.

Quality of Life (QoL) Questionnaires. Various QoL questionnaires can be used to monitor efficacy of an IL-4R antagonist, including Short-Form-36 (SF-36) questionnaire, the Euroqol-5D (EQ-5D), and the patient qualitative self-assessment.

The SF-36 is a 36-item questionnaire that measures eight multi-item dimensions of health: physical functioning (10 items) social functioning (2 items) role limitations due to physical problems (4 items), role limitations due to emotional problems (3 items), mental health (5 items), energy/vitality (4 items), pain (2 items), and general health perception (5 items). For each dimension, item scores are coded, summed, and transformed on a scale from 0 (worst possible health state measured by the questionnaire) to 100 (best possible health state). Two standardized summary scores can also be calculated from the SF-36; the physical component summary (PCS) and the mental health component summary (MCS).

The EQ-5D is a standardized health-related quality of life questionnaire developed by the EuroQol Group to provide a simple, generic measure of health for clinical and economic appraisal and inter-disease comparisons. EQ-5D, designed for self-completion by patients, consists of two parts, the EQ-5D descriptive system and the EQ VAS. The EQ-5D descriptive system comprises 5 dimensions: mobility, self-care, usual activities, pain/discomfort, and anxiety/depression; and each dimension has 3 levels: no problem, some problems, severe problems. The EQ Visual Analogue Scale (VAS) records the respondent's self-rated health on a vertical visual analogue scale. The EQ VAS ‘thermometer’ has endpoints of 100 (Best imaginable health state) at the top and 0 (Worst imaginable health state) at the bottom.

CRSsNP-Associated Biomarkers. Examples of CRSsNP-associated biomarkers include, but are not limited to, one or any combination of IgE, thymus activation regulated chemokine (TARC), eotaxin-3, periostin, IL-5, tryptase, eosinophil cationic protein, secreted P-glycoprotein, eosinophils, and combinations thereof. In certain embodiment, one or more CRSsNP-associated biomarkers may be detected from a biological sample derived from a subject. A biological sample includes, but is not limited to, one or any combination of materials taken from a patient including cultures, cells, tissues, blood, saliva, nasal secretions, nasal brushings, cerebrospinal fluid, pleural fluid, milk, lymph, sputum, semen, needle aspirates, urine, and the like. Biological samples may be obtained using any methods known in the art. For example, nasal secretion samples may be obtained from smears, blown secretions, imprints, lavage, swabs, brushes and the like. In some embodiments, an improvement in the CRSsNP symptoms is indicated by a reduction or increase (as appropriate) at week 4, week 12, week 24, or week 52 following treatment.

A normal IgE level in healthy subjects is less than about 100 kU/L (e.g., as measured using the IMMUNOCAP® assay [Phadia, Inc. Portage, MI]). Thus, embodiments include methods decreasing an elevated serum IgE level, which is a serum IgE level greater than about 100 kU/L, greater than about 150 kU/L, greater than about 500 kU/L, greater than about 1000 kU/L, greater than about 1500 kU/L, greater than about 2000 kU/L, greater than about 2500 kU/L, greater than about 3000 kU/L, greater than about 3500 kU/L, greater than about 4000 kU/L, greater than about 4500 kU/L, or greater than about 5000 kU/L, by administering to the subject a pharmaceutical composition comprising a therapeutically effective amount of an IL-4R antagonist.

TARC levels in healthy subjects are in the range of 106 ng/L to 431 ng/L, with a mean of about 239 ng/L. (An exemplary assay system for measuring TARC level is the TARC quantitative ELISA kit offered as Cat. No. DDN00 by R&D Systems, Minneapolis, MN.) Thus, the disclosure includes methods decreasing an elevated serum TARC level, which is a serum TARC (e.g., serum TARC) level greater than about 431 ng/L, greater than about 500 ng/L, greater than about 1000 ng/L, greater than about 1500 ng/L, greater than about 2000 ng/L, greater than about 2500 ng/L, greater than about 3000 ng/L, greater than about 3500 ng/L, greater than about 4000 ng/L, greater than about 4500 ng/L, or greater than about 5000 ng/L, by administering to the subject a pharmaceutical composition comprising a therapeutically effective amount of an IL-4R antagonist.

Eotaxin-3 belongs to a group of chemokines released by airway epithelial cells, which is up-regulated by the Th2 cytokines IL-4 and IL-13 (Lilly et al 1999, J. Allergy Clin. Immunol. 104: 786-790). In some embodiments, the compositions and methods include administering an IL-4R antagonist to treat patients with elevated levels of eotaxin-3, such as more than about 100 μg/ml, more than about 150 μg/ml, more than about 200 μg/ml, more than about 300 μg/ml, or more than about 350 μg/ml. Serum eotaxin-3 levels may be measured, for example, by ELISA.

Periostin is an extracellular matrix protein involved in the Th2-mediated inflammatory processes. Periostin levels are found to be up-regulated in patients with asthma (Jia et al 2012 J Allergy Clin Immunol. 130:647-654.e10. doi: 10.1016/j.jaci.2012.06.025. Epub 2012 Aug. 1). In some embodiments, the composition and methods include administering comprising administering an IL-4R antagonist to treat patients with elevated levels of periostin.

Improvement of a CRSsNP-associated parameter can be expressed as a percentage. For example, a score can be improved by 30% or more, by 40% or more, by 50% or more, by 60% or more, by 70% or more, or by 80% or more, or by 90% or more or by 100%.

Biomarker expression, as discussed above, can be assayed by detection of protein or RNA in serum. In some embodiments, RNA samples are used to determine RNA levels (non-genetic analysis), e.g., RNA levels of biomarkers; and in other embodiments, RNA samples are used for transcriptome sequencing (e.g., genetic analysis).

An “improvement in an CRSsNP-associated parameter” means an increase from baseline of one or more of NPIF, UPSIT, and/or a decrease from baseline of one or more of IgE mediated inflammatory response characteristic computed tomography (CT) findings, eosinophilic mucin. In other embodiments, improvement in an CRSsNP-associated parameter is a decrease from baseline of one or more of a SNOT-22 score, subject-assessed nasal congestion/obstruction, anterior rhinorrhea (runny nose), posterior rhinorrhea (post nasal drip) and loss of sense of smell; number of nocturnal awakenings; VAS score; Lund-Mackay score; and 3D volumetric scores; and ACQ6 score in patients with asthma. As used herein, the term “baseline,” regarding a CRSsNP-associated parameter, means the numerical value of the CRSsNP-associated parameter for a patient prior to or at the time of administration of a pharmaceutical composition of the present disclosure.

To determine whether an CRSsNP-associated parameter has “improved,” the parameter is quantified at baseline and at a time point after administration of the pharmaceutical composition of the present disclosure. For example, an CRSsNP-associated parameter may be measured at day 1, day 2, day 3, day 4, day 5, day 6, day 7, day 8, day 9, day 10, day 11, day 12, day 14, or at week 3, week 4, week 5, week 6, week 7, week 8, week 9, week 10, week 11, week 12, week 13, week 14, week 15, week 16, week 17, week 18, week 19, week 20, week 21, week 22, week 23, week 24, week 52, or longer, after the initial treatment with a pharmaceutical composition described herein. In some embodiments, the parameter is measured daily (e.g., once, or twice per day), weekly, biweekly, or monthly. In other embodiments, the parameter is measured daily, and the mean value determined over the course of a month is compared to baseline.

The difference between the value of the parameter at a particular time point following initiation of treatment and the value of the parameter at baseline is used to establish whether there has been an “improvement” in the nasal associated parameter (e.g., an increase or decrease depending on the specific parameter being measured).

### Interleukin-4 Receptor Antagonists

The methods featured herein comprise administering to a subject in need thereof a therapeutic composition comprising an IL-4R antagonist. As used herein, an “IL-4R antagonist” is any agent that binds to or interacts with IL-4R and inhibits the normal biological signaling function of IL-4R when IL-4R is expressed on a cell in vitro or in vivo. Non-limiting examples of categories of IL-4R antagonists include small molecule IL-4R antagonists, anti-IL-4R aptamers, peptide-based IL-4R antagonists (e.g., “peptibody” molecules), and antibodies or antigen-binding fragments of antibodies that specifically bind human IL-4R. According to certain embodiments, the IL-4R antagonist comprises an anti-IL-4R antibody that can be used in the context of the methods described elsewhere herein. For example, in one embodiment, the IL-4R antagonist is an antibody or antigen-binding fragment thereof that specifically binds to an IL-4R and comprises the heavy chain and light chain (complementarity determining region) CDR sequences from the heavy chain variable region (HCVR) and light chain variable region (LCVR) of SEQ ID Nos:1 and 2, respectively.

The term “human IL4R” (hIL-4R) refers to a human cytokine receptor that specifically binds to interleukin-4 (IL-4), such as IL-4Rα.

The term “antibody” refers to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2, and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments, the FRs of the anti-IL-4R antibody (or antigen-binding portion thereof) may be identical to the human germline sequences or may be naturally or artificially modified. An amino acid consensus sequence may be defined based on a side-by-side analysis of two or more CDRs.

The term “antibody” also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds to an antigen to form a complex. Antigen-binding fragments of an antibody may be derived, e.g., from full antibody molecules using any suitable standard techniques, such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add, or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment.”

An antigen-binding fragment of an antibody will typically comprise at least one variable domain. The variable domain may be of any size or amino acid composition and will generally comprise at least one CDR that is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody described herein include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-CL; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids that result in a flexible or semi-flexible linkage between adjacent variable and/or constant domains in a single polypeptide molecule, typically the hinge region may consist of between 2 to 60 amino acids, typically between 5 to 50, or typically between 10 to 40 amino acids. Moreover, an antigen-binding fragment of an antibody described herein may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, may be adapted for use in the context of an antigen-binding fragment of an antibody described herein using routine techniques available in the art.

The constant region of an antibody is important in the ability of an antibody to fix complement and mediate cell-dependent cytotoxicity. Thus, the isotype of an antibody may be selected based on whether it is desirable for the antibody to mediate cytotoxicity.

The term “human antibody” includes antibodies having variable and constant regions derived from human germline immunoglobulin sequences. The human antibodies described herein may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody” does not include antibodies in which CDR sequences derived from the germline of another mammalian species, such as a mouse, have been grafted onto human framework sequences.

The term “recombinant human antibody” includes all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created, or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. Antibodies having one or more mutations in the hinge, C_{H}2, or C_{H}3 region, which may be desirable, for example, in production, to improve the yield of the desired antibody form, are provided.

An “isolated antibody” means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody”. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” IL-4R includes antibodies that bind IL-4R or portion thereof with a K_{D} of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM, or less than about 0.5 nM, as measured in a surface plasmon resonance assay. An isolated antibody that specifically binds human IL-4R may, however, have cross-reactivity to other antigens, such as IL-4R molecules from other (non-human) species.

The anti-IL-4R antibodies useful for the methods may comprise one or more amino acid substitutions, insertions, and/or deletions (e.g., 1, 2, 3, 4, 5, 6, 7, 8, 9, or 10 substitutions and/or 1, 2, 3, 4, 5, 6, 7, 8, 9, or 10 insertions and/or 1, 2, 3, 4, 5, 6, 7, 8, 9, or 10 deletions) in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. Methods involving the use of antibodies, and antigen-binding fragments thereof, that are derived from any of the amino acid sequences disclosed herein, wherein one or more amino acids (e.g., 1, 2, 3, 4, 5, 6, 7, 8, 9, or 10 amino acids) within one or more framework and/or one or more (e.g., 1, 2, 3, 4, 5, 6, 7, 8, 9, 10, 11 or 12 with respect to the tetrameric antibody or 1, 2, 3, 4, 5 or 6 with respect to the HCVR and LCVR of an antibody) CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”), are provided. A person of ordinary skill in the art, starting with the heavy and light chain variable region sequences disclosed herein, can easily produce numerous antibodies and antigen-binding fragments that comprise one or more individual germline mutations or combinations thereof. In certain embodiments, all the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. The use of antibodies and antigen-binding fragments obtained in this general manner are encompassed within the disclosure.

Methods involving the use of anti-IL-4R antibodies comprising variants of any of the HCVR, LCVR, and/or CDR amino acid sequences disclosed herein having one or more conservative substitutions. For example, the use of anti-IL-4R antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein, are provided.

The term “surface plasmon resonance” refers to an optical phenomenon that allows for the analysis of real-time interactions by detection of alterations in protein concentrations within a biosensor matrix, for example using the BIAcore™ system (Biacore Life Sciences division of GE Healthcare, Piscataway, NJ).

The term “K_{D}” refers to the equilibrium dissociation constant of a particular antibody-antigen interaction.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 95%, or at least about 96%, 97%, 98% or 99% of the nucleotide bases, as measured by any well-known algorithm of sequence identity, such as FASTA, BLAST or Gap, as discussed below.

As applied to polypeptides, the term “substantial similarity” or “substantially similar” means that two peptide sequences, when optimally aligned, such as by the programs GAP or BESTFIT using default gap weights, share at least 95% sequence identity, or at least 98% or 99% sequence identity. In exemplary embodiments, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. (See, e.g., Pearson (1994) Methods Mol. Biol. 24: 307-331, herein incorporated by reference). Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Exemplary conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443 45, herein incorporated by reference. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. (See, e.g., GCG Version 6.1.) Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another exemplary algorithm when comparing a sequence of the disclosure to a database containing a large number of sequences from different organisms is the computer program BLAST, especially BLASTP or TBLASTN, using default parameters. (See, e.g., Altschul et al. (1990) J. Mol. Biol. 215:403-410 and Altschul et al. (1997) Nucleic Acids Res. 25:3389-402, each of which is herein incorporated by reference.)

### Preparation of Human Antibodies

Methods for generating human antibodies in transgenic mice are known in the art. Any such known methods can be used to make human antibodies that specifically bind to human IL-4R.

Using VELOCIMMUNE® technology (see, for example, U.S. Pat. No. 6,596,541, Regenerome Pharmaceuticals) or any other known method for generating monoclonal antibodies, high affinity chimeric antibodies to IL-4R are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies, or the variable domains of the light and heavy chains may be isolated directly from antigen-specific lymphocytes.

Initially, high affinity chimeric antibodies are isolated having a human variable region and a mouse constant region. The antibodies are characterized and selected for desirable characteristics, including affinity, selectivity, epitope, etc., using standard procedures known to those skilled in the art. The mouse constant regions are replaced with a desired human constant region to generate a fully human antibody described herein, for example wild-type or modified IgG1 or IgG4. While the constant region selected may vary according to specific use, high affinity antigen-binding and target specificity characteristics reside in the variable region.

In general, the antibodies that can be used in the methods described herein possess high affinities, as described above, when measured by binding to antigen either immobilized on solid phase or in solution phase. The mouse constant regions are replaced with desired human constant regions to generate the fully human antibodies described herein. While the constant region selected may vary according to specific use, high affinity antigen-binding and target specificity characteristics reside in the variable region.

In one embodiment, human antibody or antigen-binding fragment thereof that specifically binds IL-4R that can be used in the context of the methods described herein comprises the three heavy chain CDRs (HCDR1, HCDR2 and HCDR3) contained within a heavy chain variable region (HCVR) having an amino acid sequence of SEQ ID NO: 1. The antibody or antigen-binding fragment may comprise the three light chain CDRs (LCVR1, LCVR2, LCVR3) contained within a light chain variable region (LCVR) having an amino acid sequence of SEQ ID NO: 2. Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

In certain embodiments, the antibody or antigen-binding fragment thereof comprises the six CDRs (HCDR1, HCDR2, HCDR3, LCDR1, LCDR2 and LCDR3) from the heavy and light chain variable region amino acid sequence pairs (HCVR/LCVR) of SEQ ID Nos: 1 and 2.

In certain embodiments, the antibody or antigen-binding fragment thereof comprises six CDRs (HCDR1/HCDR2/HCDR3/LCDR1/LCDR2/LCDR3) having the amino acid sequences of SEQ ID Nos: 3/4/5/6/7/8.

In certain embodiments, the antibody or antigen-binding fragment thereof comprises HCVR/LCVR amino acid sequence pair of SEQ ID Nos: 1 and 2.

In certain embodiments, the antibody is dupilumab, which comprises the HCVR/LCVR amino acid sequence pair of SEQ ID Nos: 1 and 2.

In certain embodiments, the antibody sequence is dupilumab, which comprises the heavy chain/light chain amino acid sequence pair of SEQ ID Nos: 9 and 10.

Dupilumab HCVR amino acid sequence:
(SEQ ID NO: 1)
EVQLVESGGGLEQPGGSLRLSCAGSGFTFRDYAMTWVRQAPGKGLEWVSS
ISGSGGNTYYADSVKGRFTISRDNSKNTLYLQMNSLRAEDTAVYYCAKDR
LSITIRPRYYGLDVWGQGTTVTVS.
Dupilumab LCVR amino acid sequence:
(SEQ ID NO: 2)
DIVMTQSPLSLPVTPGEPASISCRSSQSLLYSIGYNYLDWYLQKSGQSPQ
LLIYLGSNRASGVPDRFSGSGSGTDFTLKISRVEAEDVGFYYCMQALQTP
YTFGQGTKLEIK.
Dupilumab HCDR1 amino acid sequence:
(SEQ ID NO: 3)
GFTFRDYA.
Dupilumab HCDR2 amino acid sequence:
(SEQ ID NO: 4)
ISGSGGNT.
Dupilumab HCDR3 amino acid sequence:
(SEQ ID NO: 5)
AKDRLSITIRPRYYGL.
Dupilumab LCDR1 amino acid sequence:
(SEQ ID NO: 6)
QSLLYSIGYNY.
Dupilumab LCDR2 amino acid sequence:
(SEQ ID NO: 7)
LGS.
Dupilumab LCDR3 amino acid sequence:
(SEQ ID NO: 8)
MQALQTPYT.
Dupilumab HC amino acid sequence:
(SEQ ID NO: 9)
EVQLVESGGGLEQPGGSLRLSCAGSGFTFRDYAMTWVRQAPGKGLEWVSS
ISGSGGNTYYADSVKGRFTISRDNSKNTLYLQMNSLRAEDTAVYYCAKDR
LSITIRPRYYGLDVWGQGTTVTVSSASTKGPSVFPLAPCSRSTSESTAAL
GCLVKDYFPEPVTVSWNSGALTSGVHTFPAVLQSSGLYSLSSVVTVPSSS
LGTKTYTCNVDHKPSNTKVDKRVESKYGPPCPPCPAPEFLGGPSVFLFPP
KPKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTTP
PVLDSDGSFFLYSRLTVDKSRWQE.G.NVFSCSVMHEALHNHYTQKSLSL
SLG
(amino acids 1-124 = HCVR; amino acids
125-451 = HC constant).
Dupilumab LC amino acid sequence:
(SEQ ID NO: 10)
DIVMTQSPLSLPVTPGEPASISCRSSQSLLYSIGYNYLDWYLQKSGQSPQ
LLIYLGSNRASGVPDRFSGSGSGTDFTLKISRVEAEDVGFYYCMQALQTP
YTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
(amino acids 1-112 = LCVR; amino acids
112-219 = LC constant).

In certain embodiments, an antibody or antigen-binding fragment thereof of the disclosure comprises light chain variable region (LCVR) and heavy chain variable region (HCVR) sequence pairs (LCVR/HCVR) selected from the group consisting of SCB-VL-39/SCB-VH-92; SCB-VL-40/SCB-VH-92; SCB-VL-41/SCB-VH-92; SCB-VL-42/SCB-VH-92; SCB-VL-43/SCB-VH-92; SCB-VL-44/SCB-VH-92; SCB-VL-44/SCB-VH-62; SCB-VL-44/SCB-VH-68; SCB-VL-44/SCB-VH-72; SCB-VL-44/SCB-VH-82; SCB-VL-44/SCB-VH-85; SCB-VL-44/SCB-VH-91; SCB-VL-44/SCB-VH-93; SCB-VL-45/SCB-VH-92; SCB-VL-46/SCB-VH-92; SCB-VL-47/SCB-VH-92; SCB-VL-48/SCB-VH-92; SCB-VL-49/SCB-VH-92; SCB-VL-50/SCB-VH-92; SCB-VL-51/SCB-VH-92; SCB-VL-51/SCB-VH-93; SCB-VL-52/SCB-VH-92; SCB-VL-52/SCB-VH-62; SCB-VL-52/SCB-VH-91; SCB-VL-53/SCB-VH-92; SCB-VL-54/SCB-VH-92; SCB-VL-54/SCB-VH-62; SCB-VL-54/SCB-VH-68; SCB-VL-54/SCB-VH-72; SCB-VL-54/SCB-VH-82; SCB-VL-54/SCB-VH-85; SCB-VL-54/SCB-VH-91; SCB-VL-55/SCB-VH-92; SCB-VL-55/SCB-VH-62; SCB-VL-55/SCB-VH-68; SCB-VL-55/SCB-VH-72; SCB-VL-55/SCB-VH-82; SCB-VL-55/SCB-VH-85; SCB-VL-55/SCB-VH-91; SCB-VL-56/SCB-VH-92; SCB-VL-57/SCB-VH-92; SCB-VL-57/SCB-VH-93; SCB-VL-57/SCB-VH-59; SCB-VL-57/SCB-VH-60; SCB-VL-57/SCB-VH-61; SCB-VL-57/SCB-VH-62; SCB-VL-57/SCB-VH-63; SCB-VL-57/SCB-VH-64; SCB-VL-57/SCB-VH-65; SCB-VL-57/SCB-VH-66; SCB-VL-57/SCB-VH-67; SCB-VL-57/SCB-VH-68; SCB-VL-57/SCB-VH-69; SCB-VL-57/SCB-VH-70; SCB-VL-57/SCB-VH-71; SCB-VL-57/SCB-VH-72; SCB-VL-57/SCB-VH-73; SCB-VL-57/SCB-VH-74; SCB-VL-57/SCB-VH-75; SCB-VL-57/SCB-VH-76; SCB-VL-57/SCB-VH-77; SCB-VL-57/SCB-VH-78; SCB-VL-57/SCB-VH-79; SCB-VL-57/SCB-VH-80; SCB-VL-57/SCB-VH-81; SCB-VL-57/SCB-VH-82; SCB-VL-57/SCB-VH-83; SCB-VL-57/SCB-VH-84; SCB-VL-57/SCB-VH-85; SCB-VL-57/SCB-VH-86; SCB-VL-57/SCB-VH-87; SCB-VL-57/SCB-VH-88; SCB-VL-57/SCB-VH-89; SCB-VL-57/SCB-VH-90; SCB-VL-57/SCB-VH-91; SCB-VL-58/SCB-VH-91; SCB-VL-58/SCB-VH-92; and SCB-VL-58/SCB-VH-93.

In certain embodiments, an antibody or antigen-binding fragment thereof of the disclosure comprises a LCVR/HCVR sequence pair of SCB-VL-44/SCB-VH-92.

In certain embodiments, an antibody or antigen-binding fragment thereof of the disclosure comprises a LCVR/HCVR sequence pair of SCB-VL-54/SCB-VH-92.

In certain embodiments, an antibody or antigen-binding fragment thereof of the disclosure comprises a LCVR/HCVR sequence pair of SCB-VL-55/SCB-VH-92.

In certain embodiments, an antibody or antigen-binding fragment thereof of the disclosure comprises an HCVR comprising an HCDR1 sequence of SCB-92-HCDR1, an HCDR2 sequence of SCB-92-HCDR2, and an HCDR3 sequence of SCB-92-HCDR3, and an LCVR comprising an LCDR1 of SCB-55-LCDR1, and LCDR2 of SCB-55-LCDR2, and an LCDR3 of SCB-55-LCDR3.

In certain embodiments, an antibody or antigen-binding fragment thereof of the disclosure comprises an HCVR comprising an HCDR1 sequence of SCB-92-HCDR1, an HCDR2 sequence of SCB-92-HCDR2, and an HCDR3 sequence of SCB-92-HCDR3, and an LCVR comprising an LCDR1 of SCB-55-LCDR1, and LCDR2 of SCB-54-LCDR2, and an LCDR3 of SCB-55-LCDR3.

In certain embodiments, an antibody or antigen-binding fragment thereof of the disclosure comprises an HCVR comprising an HCDR1 sequence of SCB-92-HCDR1, an HCDR2 sequence of SCB-92-HCDR2, and an HCDR3 sequence of SCB-92-HCDR3, and an LCVR comprising an LCDR1 of SCB-55-LCDR1, and LCDR2 of SCB-54-LCDR2, and an LCDR3 of SCB-44-LCDR3.

The antibodies recited below in Table 1 are described in more detail in U.S. Pat. No. 10,774,141, incorporated herein by reference in its entirety for all purposes.

TABLE 1

| Sequence ID | Sequence |
|---|---|
| SCB-VL-39 | EIVLTQSPGTLSLSPGERATLSCRASQSVSNSYLAWYQQKPGQAPRLL |
| | IFGASSRATGIPDRESGSGSGTDFTLTISRLEPEDFAVYYCQQYGSSPP |
| | WTFGQGTKVEIK |
| SCB-VL-40 | EIVLTQSPGTLSLSPGERATLSCRASQSVSSSYLAWYQQKPGQAPRLL |
| | IYGASSRATGIPDRESGSGSGTDFTLTISRLEPEDFAVYYCQQYGSSPP |
| | WTFGQGTKVEIK |
| SCB-VL-41 | EIVLTQSPGTLSLSPGERATLSCRASQSVSSSYLAWYQQKPGQAPRLL |
| | IFGASSRAPGIPDRESGSGSGTDFTLTISRLEPEDFAVYYCQQYGSSPP |
| | WTFGQGTKVEIK |
| SCB-VL-42 | EIVLTQSPGTLSLSPGERATLSCRASQSVSNSYLAWYQQKPGQAPRLL |
| | IYGASSRATGIPDRESGSGSGTDFTLTISRLEPEDFAVYYCQQYGSSPP |
| | WTFGQGTKVEIK |
| SCB-VL-43 | EIVLTQSPGTLSLSPGERATLSCRASQSVSNSYLAWYQQKPGQAPRLL |
| | IFGASSRAPGIPDRESGSGSGTDFTLTISRLEPEDFAVYYCQQYGSSPP |
| | WTFGQGTKVEIK |
| SCB-VL-44 | EIVLTQSPGTLSLSPGERATLSCRASQSVSSSYLAWYQQKPGQAPRLL |
| | IYGASSRAPGIPDRESGSGSGTDFTLTISRLEPEDFAVYYCQQYGSSPP |
| | WTFGQGTKVEIK |
| SCB-VL-45 | EIVLTQSPGTLSLSPGERATLSCRASQSVSSSYLAWYQQKPGQAPRLL |
| | IFGASSRATGIPDRESGSGSGTDFTLTISRLEPEDFAVYYCQQYDHSPP |
| | WTFGQGTKVEIK |
| SCB-VL-46 | EIVLTQSPGTLSLSPGERATLSCRASQSVSSSYLAWYQQKPGQAPRLL |
| | IFGASSRATGIPDRFSGSGSGTDFTLTISRLEPEDFAVYYCQQYGSSAG |
| | WTFGQGTKVEIK |
| SCB-VL-47 | EIVLTQSPGTLSLSPGERATLSCRASQSVSSSYLAWYQQKPGQAPRLL |
| | IFGASSRATGIPDRESGSGSGTDFTLTISRLEPEDFAVYYCQQYDHSAG |
| | WTFGQGTKVEIK |
| SCB-VL-48 | EIVLTQSPGTLSLSPGERATLSCRASQSVSNSYLAWYQQKPGQAPRLL |
| | IFGASSRATGIPDRFSGSGSGTDFTLTISRLEPEDFAVYYCQQYDHSPP |
| | WTFGQGTKVEIK |
| SCB-VL-49 | EIVLTQSPGTLSLSPGERATLSCRASQSVSSSYLAWYQQKPGQAPRLL |
| | IYGASSRATGIPDRFSGSGSGTDFTLTISRLEPEDFAVYYCQQYDHSPP |
| | WTFGQGTKVEIK |
| SCB-VL-50 | EIVLTQSPGTLSLSPGERATLSCRASQSVSSSYLAWYQQKPGQAPRLL |
| | IFGASSRAPGIPDRESGSGSGTDFTLTISRLEPEDFAVYYCQQYDHSPP |
| | WTFGQGTKVEIK |
| SCB-VL-51 | EIVLTQSPGTLSLSPGERATLSCRASQSVSSSYLAWYQQKPGQAPRLL |
| | IYGASSRAPGIPDRFSGSGSGTDFTLTISRLEPEDFAVYYCQQYDHSA |
| | GWTFGQGTKVEIK |
| SCB-VL-52 | EIVLTQSPGTLSLSPGERATLSCRASQSVSNSYLAWYQQKPGQAPRLL |
| | IFGASSRAPGIPDRFSGSGSGTDFTLTISRLEPEDFAVYYCQQYDHSAG |
| | WTFGQGTKVEIK |
| SCB-VL-53 | EIVLTQSPGTLSLSPGERATLSCRASQSVSNSYLAWYQQKPGQAPRLL |
| | IYGASSRATGIPDRESGSGSGTDFTLTISRLEPEDFAVYYCQQYDHSA |
| | GWTFGQGTKVEIK |
| SCB-VL-54 | EIVLTQSPGTLSLSPGERATLSCRASQSVSSSYLAWYQQKPGQAPRLL |
| | IFGASSRAPGIPDRESGSGSGTDFTLTISRLEPEDFAVYYCQQYDHSAG |
| | WTFGQGTKVEIK |
| SCB-VL-55 | EIVLTQSPGTLSLSPGERATLSCRASQSVSSSYLAWYQQKPGQAPRLL |
| | IYGASSRATGIPDRESGSGSGTDFTLTISRLEPEDFAVYYCQQYDHSA |
| | GWTFGQGTKVEIK |
| SCB-VL-56 | EIVLTQSPGTLSLSPGERATLSCRASQSVSNSYLAWYQQKPGQAPRLL |
| | IFGASSRATGIPDRESGSGSGTDFTLTISRLEPEDFAVYYCQQYDHSAG |
| | WTFGQGTKVEIK |
| SCB-VL-57 | EIVLTQSPGTLSLSPGERATLSCRASQSVSSSYLAWYQQKPGQAPRLL |
| | IFGASSRATGIPDRFSGSGSGTDFTLTISRLEPEDFAVYYCQQYGSSPP |
| | WTFGQGTKVEIK |
| SCB-VL-58 | EIVLTQSPGTLSLSPGERATLSCRASQSVSNSYLAWYQQKPGQAPRLL |
| | IYGASSRAPGIPDRESGSGSGTDFTLTISRLEPEDFAVYYCQQYDHSA |
| | GWTFGQGTKVEIK |
| SCB-VH-59 | EVQLVESGGGLVHPGGSLRLSCAGSGFTFSRNAMFWVRQAPGKGLE |
| | WVSGIGTGGATNYADSVKGRFTISRDNAKNSLYLQMNSLRAEDMA |
| | VYYCARGRYYFDYWGQGTLVTVSS |
| SCB-VH-60 | EVQLVQSGGGLVQPGGSLRLSCAGSGFTFSRNAMFWVRQAPGKGLE |
| | WVSGIGTGGATNYADSVKGRFTISRDNAKNSLYLQMNSLRAEDMA |
| | VYYCARGRYYFDYWGQGTLVTVSS |
| SCB-VH-61 | EVQLVQSGGGLVHPGGSLRLSCAASGFTFSRNAMFWVRQAPGKGLE |
| | WVSGIGTGGATNYADSVKGRFTISRDNAKNSLYLQMNSLRAEDMA |
| | VYYCARGRYYFDYWGQGTLVTVSS |
| SCB-VH-62 | EVQLVQSGGGLVHPGGSLRLSCAGSGFTFSRNAMFWVRQAPGKGLE |
| | WVSGIGTGGATSYADSVKGRFTISRDNAKNSLYLQMNSLRAEDMAV |
| | YYCARGRYYFDYWGQGTLVTVSS |
| SCB-VH-63 | EVQLVQSGGGLVHPGGSLRLSCAGSGFTFSRNAMFWVRQAPGKGLE |
| | WVSGIGTGGATNYADSVKGRFTISRDNAKNSLYLQMNSLRAEDTAV |
| | YYCARGRYYFDYWGQGTLVTVSS |
| SCB-VH-64 | EVQLVESGGGLVQPGGSLRLSCAGSGFTFSRNAMFWVRQAPGKGLE |
| | WVSGIGTGGATNYADSVKGRFTISRDNAKNSLYLQMNSLRAEDMA |
| | VYYCARGRYYFDYWGQGTLVTVSS |
| SCB-VH-65 | EVQLVESGGGLVHPGGSLRLSCAASGFTFSRNAMFWVRQAPGKGLE |
| | WVSGIGTGGATNYADSVKGRFTISRDNAKNSLYLQMNSLRAEDMA |
| | VYYCARGRYYFDYWGQGTLVTVSS |
| SCB-VH-66 | EVQLVQSGGGLVQPGGSLRLSCAASGFTFSRNAMFWVRQAPGKGLE |
| | WVSGIGTGGATNYADSVKGRFTISRDNAKNSLYLQMNSLRAEDMA |
| | VYYCARGRYYFDYWGQGTLVTVSS |
| SCB-VH-67 | EVQLVQSGGGLVHPGGSLRLSCAGSGFTFSRNAMFWVRQAPGKGLE |
| | WVSGIGTGGATSYADSVKGRFTISRDNAKNSLYLQMNSLRAEDTAV |
| | YYCARGRYYFDYWGQGTLVTVSS |
| SCB-VH-68 | EVQLVQSGGGLVHPGGSLRLSCAGSGFTFSRNAMFWVRQAPGKGLE |
| | WVSGIGTGGATNYADSVKGRFTISRDNAKNSLYLQMNSLRAEDMA |
| | VYYCARGRYYFPWWGQGTLVTVSS |
| SCB-VH-69 | EVQLVESGGGLVHPGGSLRLSCAGSGFTFSRNAMFWVRQAPGKGLE |
| | WVSGIGTGGATNYADSVKGRFTISRDNAKNSLYLQMNSLRAEDMA |
| | VYYCARGRYYFPWWGQGTLVTVSS |
| SCB-VH-70 | EVQLVQSGGGLVQPGGSLRLSCAGSGFTFSRNAMFWVRQAPGKGLE |
| | WVSGIGTGGATNYADSVKGRFTISRDNAKNSLYLQMNSLRAEDMA |
| | VYYCARGRYYFPWWGQGTLVTVSS |
| SCB-VH-71 | EVQLVQSGGGLVHPGGSLRLSCAASGFTFSRNAMFWVRQAPGKGLE |
| | WVSGIGTGGATNYADSVKGRFTISRDNAKNSLYLQMNSLRAEDMA |
| | VYYCARGRYYFPWWGQGTLVTVSS |
| SCB-VH-72 | EVQLVQSGGGLVHPGGSLRLSCAGSGFTFSRNAMFWVRQAPGKGLE |
| | WVSGIGTGGATSYADSVKGRFTISRDNAKNSLYLQMNSLRAEDMAV |
| | YYCARGRYYFPWWGQGTLVTVSS |
| SCB-VH-73 | EVQLVQSGGGLVHPGGSLRLSCAGSGFTFSRNAMFWVRQAPGKGLE |
| | WVSGIGTGGATNYADSVKGRFTISRDNAKNSLYLQMNSLRAEDTAV |
| | YYCARGRYYFPWWGQGTLVTVSS |
| SCB-VH-74 | EVQLVQSGGGLVHPGRSLRLSCAGSGFTFSRNAMFWVRQAPGKGLE |
| | WVSGIGTGGATNYADSVKGRFTISRDNAKNSLYLQMNSLRAEDMA |
| | VYYCARGRYYFDYWGQGTLVTVSS |
| SCB-VH-75 | EVQLVQSGGGLVHPGGSLRLTCAGSGFTFSRNAMFWVRQAPGKGLE |
| | WVSGIGTGGATNYADSVKGRFTISRDNAKNSLYLQMNSLRAEDMA |
| | VYYCARGRYYFDYWGQGTLVTVSS |
| SCB-VH-76 | EVQLVQSGGGLVHPGGSLRLSCAGSGFTFSRNAMHWVRQAPGKGL |
| | EWVSGIGTGGATNYADSVKGRFTISRDNAKNSLYLQMNSLRAEDMA |
| | VYYCARGRYYFDYWGQGTLVTVSS |
| SCB-VH-77 | EVQLVQSGGGLVHPGGSLRLSCAGSGFTFSRNAMFWVRQAPGE.G.L |
| | EWVSGIGTGGATNYADSVKGRFTISRDNAKNSLYLQMNSLRAEDMA |
| | VYYCARGRYYFDYWGQGTLVTVSS |
| SCB-VH-78 | EVQLVQSGGGLVHPGGSLRLSCAGSGFTFSRNAMFWVRQAPGKGLE |
| | WVSGIGTGGATNYADSVKGRFTISRDEAKNSLYLQMNSLRAEDMAV |
| | YYCARGRYYFDYWGQGTLVTVSS |
| SCB-VH-79 | EVQLVQSGGGLVHPGGSLRLSCAGSGFTFSRNAMFWVRQAPGKGLE |
| | WVSGIGTGGATNYADSVKGRFTISRDNAKNSLYLQMNSLRAGDMA |
| | VYYCARGRYYFDYWGQGTLVTVSS |
| SCB-VH-80 | EVQLVQSGGGLVHPGGSLRLSCAGSGFTFDDYAMFWVRQAPGKGL |
| | EWVSGIGTGGATNYADSVKGRFTISRDNAKNSLYLQMNSLRAEDMA |
| | VYYCARGRYYFDYWGQGTLVTVSS |
| SCB-VH-81 | EVQLVQSGGGLVQPGGSLRLSCAASGFTFSRNAMFWVRQAPGKGLE |
| | WVSGIGTGGATSYADSVKGRFTISRDNAKNSLYLQMNSLRAEDTAV |
| | YYCARGRYYFPWWGQGTLVTVSS |
| SCB-VH-82 | EVQLVESGGGLVHPGGSLRLSCAASGFTFSRNAMFWVRQAPGKGLE |
| | WVSGIGTGGATSYADSVKGRFTISRDNAKNSLYLQMNSLRAEDTAV |
| | YYCARGRYYFPWWGQGTLVTVSS |
| SCB-VH-83 | EVQLVESGGGLVQPGGSLRLSCAGSGFTFSRNAMFWVRQAPGKGLE |
| | WVSGIGTGGATSYADSVKGRFTISRDNAKNSLYLQMNSLRAEDTAV |
| | YYCARGRYYFPWWGQGTLVTVSS |
| SCB-VH-84 | EVQLVESGGGLVQPGGSLRLSCAASGFTFSRNAMFWVRQAPGKGLE |
| | WVSGIGTGGATNYADSVKGRFTISRDNAKNSLYLQMNSLRAEDTAV |
| | YYCARGRYYFPWWGQGTLVTVSS |
| SCB-VH-85 | EVQLVESGGGLVQPGGSLRLSCAASGFTFSRNAMFWVRQAPGKGLE |
| | WVSGIGTGGATSYADSVKGRFTISRDNAKNSLYLQMNSLRAEDMAV |
| | YYCARGRYYFPWWGQGTLVTVSS |
| SCB-VH-86 | EVQLVQSGGGLVHPGGSLRLSCAASGFTFSRNAMFWVRQAPGKGLE |
| | WVSGIGTGGATSYADSVKGRFTISRDNAKNSLYLQMNSLRAEDTAV |
| | YYCARGRYYFPWWGQGTLVTVSS |
| SCB-VH-87 | EVQLVQSGGGLVQPGGSLRLSCAGSGFTFSRNAMFWVRQAPGKGLE |
| | WVSGIGTGGATSYADSVKGRFTISRDNAKNSLYLQMNSLRAEDTAV |
| | YYCARGRYYFPWWGQGTLVTVSS |
| SCB-VH-88 | EVQLVESGGGLVHPGGSLRLSCAGSGFTFSRNAMFWVRQAPGKGLE |
| | WVSGIGTGGATSYADSVKGRFTISRDNAKNSLYLQMNSLRAEDTAV |
| | YYCARGRYYFPWWGQGTLVTVSS |
| SCB-VH-89 | EVQLVQSGGGLVHPGGSLRLSCAGSGFTFSRNAMFWVRQAPGKGLE |
| | WVSGIGTGGATSYADSVKGRFTISRDNAKNSLYLQMNSLRAEDTAV |
| | YYCARGRYYFPWWGQGTLVTVSS |
| SCB-VH-90 | EVQLVESGGGLVQPGGSLRLSCAASGFTFSRNAMFWVRQAPGKGLE |
| | WVSGIGTGGATNYADSVKGRFTISRDNAKNSLYLQMNSLRAEDMA |
| | VYYCARGRYYFPWWGQGTLVTVSS |
| SCB-VH-91 | EVQLVESGGGLVQPGGSLRLSCAASGFTFSRNAMFWVRQAPGKGLE |
| | WVSGIGTGGATSYADSVKGRFTISRDNAKNSLYLQMNSLRAEDTAV |
| | YYCARGRYYFDYWGQGTLVTVSS |
| SCB-VH-92 | EVQLVQSGGGLVHPGGSLRLSCAGSGFTFSRNAMFWVRQAPGKGLE |
| | WVSGIGTGGATNYADSVKGRFTISRDNAKNSLYLQMNSLRAEDMA |
| | VYYCARGRYYFDYWGQGTLVTVSS |
| SCB-VH-93 | EVQLVESGGGLVQPGGSLRLSCAASGFTFSRNAMFWVRQAPGKGLE |
| | WVSGIGTGGATSYADSVKGRFTISRDNAKNSLYLQMNSLRAEDTAV |
| | YYCARGRYYFPWWGQGTLVTVSS |
| SCB-92- | RNAMF |
| HCDR1 | |
| SCB-92- | GIGTGGATSYADSVKG |
| HCDR3 | |
| SCB-92- | GRYYFDY |
| HCDR3 | |
| SCB-55- | RASQSVSSSYLA |
| LCDR1 | |
| SCB-55- | GASSRAT |
| LCDR2 | |
| SCB-55- | QQYDHSAGWT |
| LCDR3 | |
| SCB-54- | GASSRAP |
| LCDR2 | |
| SCB-44- | QQYGSSPPWT |
| LCDR3 | |

In certain embodiments, an antibody or antigen-binding fragment thereof of the disclosure comprises light chain variable region (LCVR) and heavy chain variable region (HCVR) sequence pairs (LCVR/HCVR) selected from the group consisting of MEDI-1-VL/MEDI-1-VH through MEDI-42-VL/MEDI-42-VH.

In certain embodiments, an antibody or antigen-binding fragment thereof of the disclosure comprises a LCVR/HCVR sequence pair of MEDI-37GL-VL/MEDI-37GL-VH.

In certain embodiments, an antibody or antigen-binding fragment thereof of the disclosure comprises an HCVR comprising an HCDR1 sequence of MEDI-37GL-HCDR1, an HCDR2 sequence of MEDI-37GL-HCDR2, and an HCDR3 sequence of MEDI-37GL-HCDR3, and an LCVR comprising an LCDR1 of MEDI-37GL-LCDR1, and LCDR2 of MEDI-37GL-LCDR2, and an LCDR3 of MEDI-37GL-LCDR3.

The antibodies recited below in Table 2 are described in more detail in U.S. Pat. No. 8,877,189, incorporated herein by reference in its entirety for all purposes.

TABLE 2

| Sequence ID | Sequence |
|---|---|
| MEDI-1-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLDYWGKGTLVTVSS |
| MEDI-1-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SLSANYVFGTGTKLTVL |
| MEDI-2-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLYNWGKGTLVTVSS |
| MEDI-2-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SQPPNPLFGTGTKLTVL |
| MEDI-3-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKLLKNPWGKGTLVTVSS |
| MEDI-3-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWFG |
| | TPASNYVFGTGTKLTVL |
| MEDI-4-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLYNWGKGTLVTVSS |
| MEDI-4-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SSPPQPIFGTGTKLTVL |
| MEDI-5-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLYDWGKGTLVTVSS |
| MEDI-5-VL | QSVLTQPPSVSAAPGQKVTISCSGGSSNIGNSYVSWYQQLPGTAPKL |
| | LIYDNNKRPSGIPDRESGSKSGTSATLAITGLQTGDEADYYCGTWDT |
| | SSPPQPIFGTGTKLTVL |
| MEDI-6-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS |
| MEDI-6-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STTYHPIFGTGTKLTVL |
| MEDI-7-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWWQYWGKGTLVTVSS |
| MEDI-7-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SSPPQPIFGTGTKLTVL |
| MEDI-8-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWWQYWGKGTLVTVSS |
| MEDI-8-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STTYHPIFGTGTKLTVL |
| MEDI-9-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLYNWGKGTLVTVSS |
| MEDI-9-VL | QSVLTQPPSVSAAPGQKVTISCSGGSSNIGNSYVSWYQQLPGTAPKL |
| | LIYDNNKRPSGIPDRESGSKSGTSATLAITGLQTGDEADYYCGTWDT |
| | STTMYPLFGTGTKLTVL |
| MEDI-10-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLYDWGKGTLVTVSS |
| MEDI-10-VL | QSVLTQPPSVSAAPGQKVTISCSGGSSNIGNSYVSWYQQLPGTAPKL |
| | LIYDNNKRPSGIPDRESGSKSGTSATLAITGLQTGDEADYYCGTWDT |
| | STVLTPIFGTGTKLTVL |
| MEDI-11-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWFYDWGKGTLVTVSS |
| MEDI-11-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SPSMIPLFGTGTKLTVL |
| MEDI-12-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWFYDWGKGTLVTVSS |
| MEDI-12-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STTMYPLFGTGTKLTVL |
| MEDI-13-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLYDWGKGTLVTVSS |
| MEDI-13-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STTLQPLFGTGTKLTVL |
| MEDI-14-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLYNWGKGTLVTVSS |
| MEDI-14-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SPPTKPLFGTGTKLTVL |
| MEDI-15-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLYNWGKGTLVTVSS |
| MEDI-15-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STHRHPLFGTGTKLTVL |
| MEDI-16-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLYNWGKGTLVTVSS |
| MEDI-16-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STTYHPIFGTGTKLTVL |
| MEDI-17-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWWQHWGKGTLVTVSS |
| MEDI-17-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SPVDRPIFGTGTKLTVL |
| MEDI-18-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWWQHWGKGTLVTVSS |
| MEDI-18-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STTPMPVFGTGTKLTVL |
| MEDI-19-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWWQHWGKGTLVTVSS |
| MEDI-19-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STTYHPIFGTGTKLTVL |
| MEDI-20-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS |
| MEDI-20-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STVWEWPFGTGTKLTVL |
| MEDI-21-VH | QVQLVQSGAEVKKPGASVKVSCKASGYAFTSYYMHWARQAPGQG |
| | LEWMGIINPSGGSASYAQKFQGRVTMTRDTSTSTVYMELSSLRSED |
| | TAVYYCARGKYWMYDWGKGTLVTVSS |
| MEDI-21-VL | QSVLTQPPSVSAAPGQKVTISCSGGSSNIGNSYVSWYQQLPGTAPKL |
| | LIYDNNKRPSGIPDRFSGSKSGTSATLAITGLQTGDEAVYFCGTWDT |
| | STVWEWPFGTGTKLTVL |
| MEDI-22-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS |
| MEDI-22-VL | QPVLTQPPSVSAAPGQKVTISCSGGSSNIGNSYVSWYQQLPGTAPKL |
| | LIYDNNKRPSGIPDRFSGSKSGTSATLAITGLQTGDEADYFCGTWDT |
| | STVWEWPFGTGTKLTVL |
| MEDI-23-VH | QVQLVQSGAEVRKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS |
| MEDI-23-VL | QSVLTQPPSVSAAPGQKVTISCSGGSSNIGNNYVSWYQQLPGTAPKL |
| | LIYDNNKRPPGIPDRESGSKSGTSATLAITGLQTGDEADYYCGTWDT |
| | STVWEWPFGTGTKLTVL |
| MEDI-24-VH | QVQLVQSGAEVKKPGASVKVSCKASGYAFTSYYMHWARQAPGQG |
| | LEWMGIINPRGGSTSYAQKFQGRVTMTRDTSTSTVYMELSSLRSED |
| | TAVYYCARGKYWMYDWGKGTLVTVSS |
| MEDI-24-VL | QSVLTQPPSVSAAPGQKVTISCSGGSSNIGNSYVSWYQQLPGTAPKL |
| | LIYDNNKRPSGIPDRESGSKSGTSATLAITGLQTGDEADYFCGTWDT |
| | STVWEWPFGTGTKLTVL |
| MEDI-25-VH | QVQLVQSGAEVKKPGASVKVSCKASGYAFTSYYMHWARQAPGQG |
| | LEWMGIINPRGGSASYAQKFQGRVSMTRDTSTSTVYMELSSLRSED |
| | TAVYYCARGKYWMYDWGKGTLVTVSS |
| MEDI-25-VL | QSVLTQPPSVSAAPGQKVTISCSGGSSNIGNSYVSWYQQLPGTAPKL |
| | LIYDNNKRPSGIPDRFSGSKSGTTATLAITGLQTGDEADYYCGTWVT |
| | STVWEWPFGTGTKLTVL |
| MEDI-26-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS |
| MEDI-26-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYFCGTWDT |
| | STVWEWPFGTGTKLTVL |
| MEDI-27-VH | QVQLVQSGAEVRKPGASVKVSCKASGYAFTSYYMHWARQAPGQG |
| | LEWMGIINPSGGSTSYAQKFQGRVTMTRDTSTSTVYMELSSLRPED |
| | TAVYYCARGKYWMYDWGKGTQVTVSS |
| MEDI-27-VL | QSVLTQPPLVSAAPGQKVTISCSGGSSNIGNSYVSWYQRLPGTAPKL |
| | LIYDNNKRPSGIPDRESGSKSGTSATLAITGLQTGDEADYYCGTWDT |
| | STVWEWPFGTGTKLTVL |
| MEDI-28-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGNGTLVTVSS |
| MEDI-28-VL | LPVLTQPPSVSAAPGQKVTISCSGGSSSIGNSYVSWYQQLPGAAPKL |
| | LIYDNNKRPSGIPDRFSGFRSGTSATLAITGLQTGDEADYYCGTWDT |
| | SPVWEWPFGTGTKLTVL |
| MEDI-29-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTRVTVSS |
| MEDI-29-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SPVWEWPFGTGTKLTVL |
| MEDI-30-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS |
| MEDI-30-VL | QSVLTQPPSVSAAPGQKVTISCSGGSSNIGNSYVSWYQRLPGAAPKL |
| | LIYDNNKRPSGIPDRFSGSKSGTSATLAITGLQTGDEADYYCGTWDT |
| | STVWEWPFGTGTKLTVL |
| MEDI-31-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS |
| MEDI-31-VL | QSVLTQPPSVSAAPGQKVTISCSGGSSSIGNSYVSWYQQLPGTAPKL |
| | LIYDNNKRPSGIPDRFSGSKSGTSATLAITGLQTGDEADYYCGTWAT |
| | SPVWEWPFGTGTKLTVL |
| MEDI-32-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS |
| MEDI-32-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYFCGTWDT |
| | STAWEWPFGTGTKLTVL |
| MEDI-33-VH | QVQLVQSGAEEKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS |
| MEDI-33-VL | QSALTQPPSVSAAPGQKVTISCSGGSSNIGNSYVSWYQQLPGTAPKL |
| | LIYDNNKRPSGIPDRESGSKSGTSATLAITGLQTGDEADYFCGTWDT |
| | STVWEWPFGTGTKLTVL |
| MEDI-34-VH | QVQLVQSGAEVKKPGASVKVSCKASGYAFTSYYMHWARQAPGQG |
| | LEWMGIINPSGGSTSYAQKFQGRVSMTRDTSTSTVYMELSSLRSEDT |
| | AVYYCARGKYWMYDWGKGTLVTVSS |
| MEDI-34-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYFCGTWDT |
| | STVWEWPFGTGTKLTVL |
| MEDI-35-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS |
| MEDI-35-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SPVWEWPFGTGTKLTVL |
| MEDI-36-VH | QVQLVQSGAEVKKPGASVKVSCKASGYAFTSYYMHWARQAPGQG |
| | LEWMGIINPSGGSASYAQKFQGRVTMTRDTSTSTVYMELSSLRSED |
| | TAVYYCARGKYWMYDWGKGTLVTVSS |
| MEDI-36-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDS |
| | STVWEWPFGTGTKLTVL |
| MEDI-37-VH | QVQLVQSGAEVKKPGASVKVSCKASGYAFTSYYMHWARQAPGQG |
| | LEWMGIINPRGGSTSYAQKFQGRVAMTRDTSTSTVYMELSSLRPED |
| | TAVYYCARGKYWMYDWGKGTLVTVSS |
| MEDI-37-VL | QSVLTQPPSVSAAPGQKVTISCSGGGSSIGNSYVSWYQQLPGTAPKL |
| | LIYDNNKRPSGVPDRFSGSKSGTSATLAITGLQTGDEADYYCGTWD |
| | TSPVWEWPFGTGTKLTVL |
| MEDI-38-VH | QVQLVQSGAEVKKPGASVKVSCKASGYAFTSYYMHWARQAPGQG |
| | LEWMGIINPSGGSASYAQKFQGRVTMTRDTSTSTVYMELSSLRSED |
| | TAVYYCARGKYWMYDWGKGTLVTVSS |
| MEDI-38-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYFCGTWDT |
| | STVWEWPFGTGTKLTVL |
| MEDI-39-VH | QVQLVQSGAEVKKPGASVKVSCKASGYAFTSYYMHWARQAPGQG |
| | LEWMGIINPRGGSTSYAQKFQGRVTMTRDTSTSTVYMELSSLRSED |
| | TAVYYCARGKYWMYDWGKGTLVTVSS |
| MEDI-39-VL | QSVLTQPPSVSAAPGQKVTISCSGGSSNIGNSYVSWYQQLPGTAPKL |
| | LIYDNNKRPSGIPDRESGSKSGTSATLAITGLQTGDEADYYCGTWDT |
| | STAWEWPFGTGTKLTVL |
| MEDI-40-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS |
| MEDI-40-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDS |
| | STVWEWPFGTGTKLTVL |
| MEDI-41-VH | QVQLVQSGAEVRKPGASVKVSCKASGYAFTSYYMHWARQAPGQG |
| | LEWMGIINPSGGSTSYAQKFQGRVTMTRDTSTSTVYMELSSLRPED |
| | TAVYYCARGKYWMYDWGKGTLVTVSG |
| MEDI-41-VL | QSVLTQPPSVSAAPGQKVTISCSGGSTNIGNSYVSWYQRLPGTAPKL |
| | LIYDNNKRPPGIPDRFSGSKSGTSATLAITGLQTGDEADYYCGTWDT |
| | STVWEWPFGTGTKLTVL |
| MEDI-42-VH | QVQLVQSGAEVKKPGASVKVSCKASGYAFTSYYMHWARQAPGQG |
| | LEWVGIINPSGGSTSYAQKFQGRVTMTRDTSTSTVYMELSSLRSGDT |
| | AVYYCARGKYWMYDWGKGTLVTVSS |
| MEDI-42-VL | QAVLTQPPSVSAAPGQKVTISCSGGSSNIGNSYVSWYQRLPGAAPKL |
| | LIYDNNKRPSGIPDRESGSKSGTSATLAITGLQTGDEADYYCGTWDT |
| | STGWEWPFGTGTKLTVL |
| MEDI-37GL- | QVQLVQSGAEVKKPGASVKVSCKASGYAFTSYYMHWVRQAPGQG |
| VH | LEWMGIINPRGGSTSYAQKFQGRVTMTRDTSTSTVYMELSSLRSED |
| | TAVYYCARGKYWMYDWGKGTLVTVSS |
| MEDI-37GL- | QSVLTQPPSVSAAPGQKVTISCSGGGSSIGNSYVSWYQQLPGTAPKL |
| VL | LIYDNNKRPSGIPDRESGSKSGTSATLGITGLQTGDEADYYCGTWDT |
| | SPVWEWPFGTGTKLTVL |
| MEDI-37GL- | SYYMH |
| HCDR1 | |
| MEDI-37GL- | IINPRGGSTSYAQKFQG |
| HCDR2 | |
| MEDI-37GL- | GKYWMYD |
| HCDR3 | |
| MEDI-37GL- | SGGGSSIGNSYVS |
| LCDR1 | |
| MEDI-37GL- | DNNKRPS |
| LCDR2 | |
| MEDI-37GL- | GTWDTSPVWEWP |
| LCDR3 | |

In certain embodiments, an antibody or antigen-binding fragment thereof of the disclosure comprises a LCVR/HCVR sequence pair of AJOU-90-VL/AJOU-83-VH.

In certain embodiments, an antibody or antigen-binding fragment thereof of the disclosure comprises an HCVR comprising an HCDR1 sequence of AJOU-84-HCDR1, an CHDR2 sequence of AJOU-85-HCDR2, and an HCDR3 sequence of AJOU-32-HCDR3, and an LCVR comprising an LCDR1 of AJOU-96-LCDR1, and LCDR2 of AJOU-60-LCDR2, and an LCDR3 of AJOU-68-LCDR3.

The antibodies recited below in Table 3 are described in more detail in WO2020/096381 and Kim et al. (Scientific Reports. 9: 7772. 2019), incorporated herein by reference in their entireties for all purposes.

TABLE 3

| Sequence ID | Sequence |
|---|---|
| AJOU-1-VH | EVQLLESGGGLVQPGGSLRLSCAVSGFTFSNYAMSWVRQAPGKGL |
| | EWVSAISSGGGNIYYADSVKGRFTISRDNSKNTLYLQMNSLRAEDT |
| | AVYYCAKLRRYFDYWGQGTLVTVSS |
| AJOU-2-VH | EVQLLESGGGLVQPGGSLRLSCAASGFTFSDYAMSWVRQAPGKGL |
| | EWVSAISSGGSSIYYADSVKGRFTISRDNSKNTLHLQMNSLRAEDT |
| | AVYYCARGPQRSATAVFDYWGQGTLVTVSS |
| AJOU-3-VH | EVQLLESGGGLVQPGGSLRLSCAASGFTFSNYAMSWVRQAPGKGL |
| | EWVSWISPNSGNIYYADSVKGRFTISRDNSKNTLYLQMNSLRAEDT |
| | AVYYCARRPLSAAWSHSSYYNAMDVWGQGTLVTVSS |
| AJOU-4-VH | EVQLLESGGGLVQPGGSLRLSCAASGFTFSGYAMSWVRQAPGKGL |
| | EWVSLISHSGSNTYYADSVKGRFTISRDNSKNTLYLQMNSLRAEDT |
| | AVYYCARPHRAFDYWGQGTLVTVSS |
| AJOU-5-VH | EVQLLESGGGLVQPGGSLRLSCAASGFTFSNYAMSWVRQAPGKGL |
| | EWVSGISHGSGSIYYADSVKGRFTISRDNSKNTLYLQMNSLRAEDT |
| | AVYYCARPHRAFDYWGQGTLVTVSS |
| AJOU-6-VH | EVQLLESGGGLVQPGGSLRLSCAASGFTFSNYAMSWVRQAPGKGL |
| | EWVSGISHGNGSIYYADSVKGRFTISRDNSKNTLYLQMNSLRAEDT |
| | AVYYCAKTGRHFDYWGQGTLVTVSS |
| AJOU-7-VH | EVQLLESGGGLVQPGGSLRLSCAASGFTFSNYAMSWVRQAPGKGL |
| | EWVSSISPSGSSIYYADSVKGRFTISRDNSKNTLYLQMNSLRAEDTA |
| | VYYCARSYRAFDYWGQGTLVTVSS |
| AJOU-8-VH | EVQLLESGGGLVQPGGSLRLSCAASGFTFSNYAMSWVRQAPGKGL |
| | EWVSAISPSGGSIYYADSVKGRFTISRDNSKNTLYLQMNSLRAEDT |
| | AVYYCARAKRAFDYWGQGTLVTVSS |
| AJOU-9-VH | EVQLLESGGGLVQPGGSLRLSCAASGFTFSNYAMSWVRQAPGKGL |
| | EWVSAISPGSGSTYYADSVKGRFTISRDNSKNTLYLQMNSLRAEDT |
| | AVYYCAKFRRHFDYWGQGTLVTVSS |
| AJOU-10-VH | EVQLLESGGGLVQPGGSLRLSCAASGFTFSNYAMSWVRQAPGKGL |
| | EWVSAISSGGGNIYYADSVKGRFTISRDNSKNTLYLQMNSLRAEDT |
| | AVYYCARVHRAFDYWGQGTLVTVSS |
| AJOU-69-VH | EVQLLESGGGLVQPGGSLRLSCAASGFTFSNYAMSWVRQAPGKGL |
| | EWVSAITSSGRSIYYADSVKGRFTISRDNSKNTLYLQMNSLRAEDT |
| | AVYYCARVHRAFDYWGQGTLVTVSS |
| AJOU-70-VH | EVQLLESGGGLVQPGGSLRLSCAASGFTFSNYAMSWVRQAPGKGL |
| | EWVSAITSSGANIYYADSVKGRFTISRDNSKNTLYLQMNSLRAEDT |
| | AVYYCARVHRAFDYWGQGTLVTVSS |
| AJOU-71-VH | EVQLLESGGGLVQPGGSLRLSCAASGFTFSNYAMSWVRQAPGKGL |
| | EWVSAITSSGGNIYYADSVKGRFTISRDNSKNTLYLQMNSLRAEDT |
| | AVYYCARVHRAFDYWGQGTLVTVSS |
| AJOU-72-VH | EVQLLESGGGLVQPGGSLRLSCAASGFTFSNYAMSWVRQAPGKGL |
| | EWVSAITAGGGSIYYADSVKGRFTISRDNSKNTLYLQMNSLRAEDT |
| | AVYYCARVHRAFDYWGQGTLVTVSS |
| AJOU-83-VH | EVQLLESGGGLVQPGGSLRLSCAASGFTFSRHAMAWVRQAPGKGL |
| | EWVSAITSSGRSIYYADSVKGRFTISRDNSKNTLYLQMNSLRAEDT |
| | AVYYCARVHRAFDYWGQGTLVTVSS |
| AJOU-33-VL | QSVLTQPPSASGTPGQRVTISCSGSSSNIGNNYVNWYQQLPGTAPK |
| | LLIYDNSHRPSGVPDRFSGSKSGTSASLAISGLRSEDEADYYCGTW |
| | DASLSAYVFGGGTKLTVL |
| AJOU-34-VL | QSVLTQPPSASGTPGQRVTISCSGSSSNIGNNNVSWYQQLPGTAPKL |
| | LIYANSKRPSGVPDRESGSKSGTSASLAISGLRSEDEADYYCGSWD |
| | DSLSAYVFGGGTKLTVL |
| AJOU-35-VL | QSVLTQPPSAPGTPGQRVTISCTGSSSNIGSNSVNWYQQLPGTAPKL |
| | LIYDDSHRPSGVPDRFSGSKSGTSASLAISGLRSEDEADYYCDAWD |
| | SSLSAYVFGGGTKLTVL |
| AJOU-36-VL | QSVLTQPPSASGTPGQRVTLSCTGSSSNIGSNYVSWYQQLPGTAPK |
| | LLIYADSQRPSGVPDRFSGSKSGTSASLAISGLRSEDEADYYCGTW |
| | DDSLSGYVFGGGTKLTVL |
| AJOU-37-VL | QSVLTQPPSASGTPGQRVTISCSSSSSNIGSNYVSWYQQLPGTAPKL |
| | LIYSDSHRPSGVPDRESGSKSGTSASLAISGLRSEDEADYYCGSWDY |
| | SLSAYVFGGGTKLTVL |
| AJOU-38-VL | QSVLTQPPSASGTPGQRVTISCTGSSSNIGNNTVSWYQQLPGTAPKL |
| | LIYDNSHRPSGVPDRFSGSKSGTSASLAISGLQSEDEADYYCGSWD |
| | YSLSAYVFGGGTKLTVL |
| AJOU-39-VL | QSVLTQPPSASGTPGQRVTISCTGSSSNIGNNDVNWYQQLPGTAPK |
| | LLIYYDSQRPSGVPDRESGSKSGTSASLAISGLRSEDEADYYCATW |
| | DASLSAYVFGGGTKLTVL |
| AJOU-40-VL | QSVLTQPPSASGTPGQRVTISCSGSSSNIGSNAVNWYQQLPGTAPKL |
| | LIYYDNQRPSGVPDRFSGSKSGTSASLAISGLRSEDEADYYCGTWD |
| | DSLNGYVFGGGTKLTVL |
| AJOU-41-VL | QSVLTQPPSASGTPGQRVTISCSGSSSNIGNNAVTWYQQLPGTAPK |
| | LLIYDDSHRPSGVPDRESGSKSGTSASLAISGLRSEDEADYYCGSW |
| | DYSLSAYVFGGGTKLTVL |
| AJOU-42-VL | QSVLTQPPSASGTPGQRVTISCSGSSSNIGSNTFNWYQQLPGTAPKL |
| | LIYADSHRPSGVPDRFSGSKSGTSASLAISGLRSEDEADYYCGTWD |
| | YSLSGYVLGGGTKLTVL |
| AJOU-77-VL | QSVLTQPPSASGTPGQRVTISCSGSSSNIGSNTFNWYQQLPGTAPKL |
| | LIYADSHRPSGVPDRESGSKSGTSASLAISGLRSEDEADYYCGTWD |
| | YSLSGYVLGGGTKLTVL |
| AJOU-78-VL | QSVLTQPPSASGTPGQRVTISCSGSSSNIGSNTENWYQQLPGTAPKL |
| | LIYADSHRPSGVPDRFSGSKSGTSASLAISGLRSEDEADYYCGTWD |
| | YSLRGYVLGGGTKLTVL |
| AJOU-79-VL | QSVLTQPPSASGTPGQRVTISCSGSSSNIGSNTFNWYQQLPGTAPKL |
| | LIYADSHRPSGVPDRFSGSKSGTSASLAISGLRSEDEADYYCGYWD |
| | YSLSGYVLGGGTKLTVL |
| AJOU-80-VL | QSVLTQPPSASGTPGQRVTISCSGSSSNIGSNTFNWYQQLPGTAPKL |
| | LIYADSHRPSGVPDRESGSKSGTSASLAISGLRSEDEADYYCGTWD |
| | YSLSGYVLGGGTKLTVL |
| AJOU-86-VL | QSVLTQPPSASGTPGQRVTISCSGSSANSRTDGFNWYQQLPGTAPK |
| | LLIYADSHRPSGVPDRFSGSKSGTSASLAISGLRSEDEADYYCGTW |
| | DYSLSGYVLGGGTKLTVLG |
| AJOU-87-VL | QSVLTQPPSASGTPGQRVTISCSGSAQFGSRDNFNWYQQLPGTAPK |
| | LLIYADSHRPSGVPDRESGSKSGTSASLAISGLRSEDEADYYCGTW |
| | DYSLSGYVLGGGTKLTVLG |
| AJOU-88-VL | QSVLTQPPSASGTPGQRVTISCSGSTKQMHNYQFNWYQQLPGTAP |
| | KLLIYADSHRPSGVPDRFSGSKSGTSASLAISGLRSEDEADYYCGT |
| | WDYSLSGYVLGGGTKLTVLG |
| AJOU-89-VL | QSVLTQPPSASGTPGQRVTISCSGSLLRGENLQFNWYQQLPGTAPK |
| | LLIYADSHRPSGVPDRFSGSKSGTSASLAISGLRSEDEADYYCGTW |
| | DYSLSGYVLGGGTKLTVLG |
| AJOU-90-VL | QSVLTQPPSASGTPGQRVTISCSGSPLFPDSGSFNWYQQLPGTAPKL |
| | LIYADSHRPSGVPDRFSGSKSGTSASLAISGLRSEDEADYYCGTWD |
| | YSLSGYVLGGGTKLTVLG |
| AJOU-91-VL | QSVLTQPPSASGTPGQRVTISCSGSAALDLSPSFNWYQQLPGTAPKL |
| | LIYADSHRPSGVPDRFSGSKSGTSASLAISGLRSEDEADYYCGTWD |
| | YSLSGYVLGGGTKLTVLG |
| AJOU-84- | RHAMA |
| HCDR1 | |
| AJOU-85- | AITSSGRSIYYADSVKG |
| HCDR2 | |
| AJOU-32- | VHRAFDY |
| HCDR3 | |
| AJOU-96- | SGSPLFPDSGSFN |
| LCDR1 | |
| AJOU-60- | ADSHRPS |
| LCDR2 | |
| AJOU-68- | GTWDYSLSGYV |
| LCDR3 | |

In certain embodiments, an antibody or antigen-binding fragment thereof of the disclosure comprises light chain variable region (LCVR) and heavy chain variable region (HCVR) sequence pairs (LCVR/HCVR) selected from the group consisting of 11/3, 27/19, 43/35, 59/51, 75/67, 91/83, 107/99, 123/115, 155/147, and 171/163.

The antibodies recited below in Table 4 are described in more detail in U.S. Pat. Nos. 7,605,237 and 7,608,693, incorporated herein by reference in their entireties for all purposes.

TABLE 4

| Sequence ID | Sequence |
|---|---|
| REGN-VH-3 | QVQLVQSGAEVKKPGASVKVSCKASGYTFTNYGISWVRQAPGQG |
| | LEWMGWISVYNGKTNYAQKLQGRVTMTTDTSTTTAYMEMRSLR |
| | SDDTAVYYCARGSGYDLDYWGQGTLVSVSS |
| REGN-VH-19 | EVQLVESGGGLVQPGGSLRLSCAASGFTFSSFWMTWVRQAPGKG |
| | LEWVANIKQDGSEKYYVDSVKGRFTISRDNAKNSLYLQMNSLRA |
| | EDTAVYYCARDPGRTMVRGGIRYYYGMDVWGQGTTVTVSS |
| REGN-VH-35 | EVKLAESGGGLVQPGGSLRLSCAASGFTFSSHWMNWVRQAPGKG |
| | LEWVANIKQDGSDKYYVDSVKGRFTISRDNAKNSLYLQLNSLIAE |
| | DTAVYYCARDRGVRPPRGAFDIWGQGTMVTVSS |
| REGN-VH-51 | QVQLVQSGAEVKKPGASVKVSCKASGYTFNSYGISWVRQAPGQG |
| | LEWMGWIRTYNGNTNYAQKLQGRVTMTTDTSTSTAYMELRSLR |
| | SDDTAVYYCARDEARIVVAGTTPYYYGMDVWGQGTTVTVSS |
| REGN-VH-67 | QVQLVESGGGLVQPGGSLRLSCAVSGFTISDHYMSWIRQAPGKGL |
| | EWISYISSSGSKIYYADSVKGRFTISRDNAKNSLFLQMNSLRAEDT |
| | AVYYCARTRQLVGDYWGQGTLVTVSS |
| REGN-VH-83 | EVQLVESGGGLVQPGRSLRLSCAASGFTFDNYAMHWVRQAPGK |
| | GLEWVSGIRWNSGSIGYADSVKGRFTISRDNAKNSLYLQMNSLRA |
| | EDTALYYCAKE.G.GYSGYRPGPFFDYWGQGTLVTVSS |
| REGN-VH-99 | QVQLVQSGAEVKKPGASVKVSCKASGYTFTNYGISWVRQAPGQG |
| | LEWMGWISVYNGHTNYAQKLQGRVTMTTDTSTSTAYMELRSLR |
| | SDDTAVYYCARGSGYDFDSWGQGTLVTVSS |
| REGN-VH-115 | QVQLVQSGAEVKKPGASVKVSCKASRYTFTSYDINWVRQATGQG |
| | LEWMGWMNPNSGNTGYAQKFQGRVTMTRNTSTSTAYMELSSLR |
| | SEDTAVYYCARVRRFFDYWGQGTLVTVSS |
| REGN-VH-147 | QVQLVQSGPEVKKPGASVKVSCKASGYTFTNYGISWVRQAPGQG |
| | LEWMGWISVYNGNINYAQKLQGRVTMTTDTSTSTAYMDLRSLRS |
| | DDTAVYYCARGSGYDFDYWGQGTLVTVSS |
| REGN-VH-163 | QVQLVQSGAEVKKPGASVKVSCKDSAYTFNRYGISWVRQAPGQG |
| | LEWMGWISAYTGNTVYAQKLQGRVTMTTDNSTSTAYMELRSLR |
| | SDDTAVYYCARDKSIFGVVRGFDYWGQGTLVTVSS |
| REGN-VL-11 | AIQMTQSPSSLSASVGDRVTITCRASQGIRNALGWYQQKPGKAPK |
| | LLIYAASSLQSGVPSRFSGSGSGTDFTLTFSSLQPEDFATYYCLQDF |
| | NYPYTFGQGTKLEIK |
| REGN-VL-27 | DIQMTQSPSSVSASVGDRVTISCRASQGVSSWLAWYQQKPGNAP |
| | KLLISAASSIQSGVPSRFSGSGSGTDFTLTISSLQPEDFATYYCQQA |
| | NSFPLTFGGGTKVEIK |
| REGN-VL-43 | DIQMTQSPSSVSASVGDRVTITCRASQGISSWLAWYQQKPGKAPK |
| | LLIYAASSFQSGVPSRFSGSGSGTDFTLTISSLQPEDFATYFCQQAN |
| | SFPLTFGGGTTVEIK |
| REGN-VL-59 | DIQMTQSPSSVSASVGDRVTITCRASQDISIWLAWYQQSPGKAPKL |
| | LINVASRLQSGVPSRFSGSGSGTDFTLTINSLQPEDFVTYYCQQAN |
| | SFPITFGQGTRLATK |
| REGN-VL-75 | DIQLTQSPSFLSASVGDRVTITCWASQGISSYLAWYQQKPGKAPKL |
| | LIFAASTLQSGVPSRFSGSGSGTEFTLTISSLQPEDFATYYCQQLNS |
| | YPLTFGGGTKVEIR |
| REGN-VL-91 | EIVMTQSPATLSVSPGERATLSCRASQSVNYNLAWYQHKPGQAPR |
| | LLIYGASTRATGIPARFSGSGSGTEFTLTISSLQSEDFAVYYCQQYN |
| | NWPLTFGGGTKVEIK |
| REGN-VL-107 | AIQMTQSSSSLSASVGDRVTITCRASQAIRNALGWYQQKPGKAPK |
| | VLIYAASSLQSGIPSRFSGSGSGTDFTLTISSLQPEDFATYYCLQDY |
| | DYPYTFGQGTKLEIK |
| REGN-VL-123 | DIQLTQSPSFLSASVGDRVTITCWASQGIISYLAWYQQKPGKAPKL |
| | LIYAASTLHSGVPSRFSGSGSGTEFTLTISSLQPEDFATYYCHQLKS |
| | YPITFGQGTRLEIK |
| REGN-VL-155 | AIQMTQSPSSLSASVGDRVTITCRASQDIRNALGWYQQKPGKAPK |
| | LLIYAASSLQSGVPSRFSGSASGTDFTLTISSLQPEDFAAYYCLQDY |
| | NYPYTFGQGTKLEIK |
| REGN-VL-171 | EIVMTQSPVTLSLSPGERATLPCRASQSVSSSLAWYQQKAGQSPRL |
| | LIYGASTRATGIPARFSGSGSGTEFTLTISNLQSEDFAVYYCQQYN |
| | NWPLTFGGGTKVEIK |

The antibodies recited below in Table 5 are described in more detail in WO2022/052974, incorporated herein by reference in its entirety for all purposes.

TABLE 5

| | |
|---|---|
| EVQLLESGGGLVQPGGSLRLSCAASGFTLSSYAMHWVR | STSA-C27-VH |
| QAPGKGLEYVSGISSNGGSTYYANSVKGRFTISRDNPKN | |
| TLFLQMSSLRAEDTAVYYCVRVKVGYRGGMDVWGQG | |
| TTVTVSS | |
| EVQLLESGGGLVQPGGSLRLSCAASGFTLSSYAMHWVR | STSA-C27-6-33-VH |
| QAPGKGLEYVSGISPSGSSTYYANSVKGRFTISRDNPKNT | |
| LFLQMSSLRAEDTAVYYCVRSKVRYRGGMDVWGQGTT | |
| VTVSS | |
| EVQLLESGGGLVQPGGSLRLSCAASGFTLSSYAMHWVR | STSA-C27-7-33-VH |
| QAPGKGLEYVSGISPSGVSTYYANSVKGRFTISRDNPKN | |
| TLFLQMSSLRAEDTAVYYCVRVKVKYRGGMDVWGQG | |
| TTVTVSS | |
| EVQLLESGGGLVQPGGSLRLSCAASGFTLSSYAMHWVR | STSA-C27-24-56- |
| QAPGKGLEYVSGISPTSGSTYYANSVKGRFTISRDNPKNT | VH |
| LFLQMSSLRAEDTAVYYCVRVKVRYRGGMDVWGQGTT | |
| VTVSS | |
| EVQLLESGGGLVQPGGSLRLSCAASGFTLSSYAMHWVR | STSA-C27-47-56- |
| QAPGKGLEYVSGISPTGTSTYYANSVKGRFTISRDNPKNT | VH |
| LFLQMSSLRAEDTAVYYCVRVKGAYRGGMDVWGQGT | |
| TVTVSS | |
| EVQLLESGGGLVQPGGSLRLSCAASGFTLSSYAMHWVR | STSA-C27-33-33- |
| QAPGKGLEYVSGISSSGSSTYYANSVKGRFTISRDNPKNT | VH |
| LFLQMSSLRAEDTAVYYCVRVKVAYRGGMDVWGQGT | |
| TVTVSS | |
| EVQLLESGGGLVQPGGSLRLSCAASGFTLSSYAMHWVR | STSA-C27-56-56- |
| QAPGKGLEYVSGISPSSTSTYYANSVKGRFTISRDNPKNT | VH |
| LFLQMSSLRAEDTAVYYCVRVKVLYRGGMDVWGQGTT | |
| VTVSS | |
| EVQLLESGGGLVQPGGSLRLSCAASGFTLSSYAMHWVR | STSA-C27-78-78- |
| QAPGKGLEYVSGISPSSASTYYANSVKGRFTISRDNPKNT | VH |
| LFLQMSSLRAEDTAVYYCVRVKSKYRGGMDVWGQGTT | |
| VTVSS | |
| EVQLLESGGGLVQPGGSLRLSCAASGFTLSSYAMHWVR | STSA-C27-82-58- |
| QAPGKGLEYVSGISGNSASTYYANSVKGRFTISRDNPKN | VH |
| TLFLQMSSLRAEDTAVYYCVRVKLKYRGGMDVWGQGT | |
| TVTVSS | |
| EVQLLESGGGLVQPGGSLRLSCAASGFTLSSYAMHWVR | STSA-C27-54-54- |
| QAPGKGLEYVSGISHSGTSTYYANSVKGRFTISRDNPKN | VH |
| TLFLQMSSLRAEDTAVYYCVRVRVLYRGGMDVWGQGT | |
| TVTVSS | |
| EVQLLESGGGLVQPGGSLRLSCAASGFTLSSYAMHWVR | STSA-C27-36-36- |
| QAPGKGLEYVSGISPSGVSTYYANSVKGRFTISRDNPKN | VH |
| TLFLQMSSLRAEDTAVYYCVRVKVKYRGGMDVWGQG | |
| TTVTVSS | |
| EVQLLESGGGLVQPGGSLRLSCAASGFTLSSYAMHWVR | STSA-C27-53-53- |
| QAPGKGLEYVSGISSNGGSTYYANSVKGRFTISRDNPKN | VH |
| TLFLQMSSLRAEDTAVYYCVRVFVRYRGGMDVWGQGT | |
| TVTVSS | |
| EVQLLESGGGLVQPGGSLRLSCAASGFTLSSYAMHWVR | STSA-C27-67-67- |
| QAPGKGLEYVSGISPTSASTYYANSVKGRFTISRDNPKNT | VH |
| LFLQMSSLRAEDTAVYYCVRVKGRYRGGMDVWGQGTT | |
| VTVSS | |
| EVQLLESGGGLVQPGGSLRLSCAASGFTLSSYAMHWVR | STSA-C27-55-55- |
| QAPGKGLEYVSGISPTGGSTYYANSVKGRFTISRDNPKN | VH |
| TLFLQMSSLRAEDTAVYYCVRVKGRYRGGMDVWGQGT | |
| TVTVSS | |
| EVQLLESGGGLVQPGGSLRLSCAASGFTLSSYAMHWVR | STSA-C27-59-59- |
| QAPGKGLEYVSGISHSGNSTYYANSVKGRFTISRDNPKN | VH |
| TLFLQMSSLRAEDTAVYYCVRVKRRYRGGMDVWGQGT | |
| TVTVSS | |
| EVQLLESGGGLVQPGGSLRLSCAASGFTLSSYAMHWVR | STSA-C27-58-58- |
| QAPGKGLEYVSGISPSSNSTYYANSVKGRFTISRDNPKNT | VH |
| LFLQMSSLRAEDTAVYYCVRVKVRYRGGMDVWGQGTT | |
| VTVSS | |
| EVQLLESGGGLVQPGGSLRLSCAASGFTLSSYAMHWVR | STSA-C27-52-52- |
| QAPGKGLEYVSGISSSGSSTYYANSVKGRFTISRDNPKNT | VH |
| LFLQMSSLRAEDTAVYYCVRVKPAYRGGMDVWGQGTT | |
| VTVSS | |
| EVQLLESGGGLVQPGGSLRLSCAASGFTLSSYAMHWVR | STSA-C27-Y2-Y2- |
| QAPGKGLEYVSGISYSSASTYYANSVKGRFTISRDNPKN | VH |
| TLFLQMSSLRAEDTAVYYCVRVKVRYRGGMDVWGQGT | |
| TVTVSS | |
| ETTLTQSPDTLPLSPGDRASLSCRASQSVSSAYLAWYQQ | STSA-C27-VL |
| KPGQAPRLLIYGTSRRATGVPGRFSGSGSGTDFTLTISRL | |
| EPEDFAVYYCQLYGSSSVTFGQGTKLEIK | |
| EIVLTQSPGTLSLSPGERATLSCRASQGISSAYLAWYQQK | STSA-C27-6-33-VL |
| PGQAPRLLIYGTSRRATGIPDRESGSGSGTDFTLTISRLEP | |
| EDFAVYYCQLYGATSVTFGQGTKLEIK | |
| EIVLTQSPGTLSLSPGERATLSCRASQGISSAYLAWYQQK | STSA-C27-7-33-VL |
| PGQAPRLLIYGTSRRATGIPDRESGSGSGTDFTLTISRLEP | |
| EDFAVYYCQLYGATSVTFGQGTKLEIK | |
| EIVLTQSPGTLSLSPGERATLSCRASQSVSSAYLAWYQQ | STSA-C27-24-56- |
| KPGQAPRLLIYGTSRRATGIPDRFSGSGSGTDFTLTISRLE | VL |
| PEDFAVYYCQLYGASSVTFGQGTKLEIK | |
| EIVLTQSPGTLSLSPGERATLSCRASQSVSSAYLAWYQQ | STSA-C27-47-56- |
| KPGQAPRLLIYGTSRRATGIPDRFSGSGSGTDFTLTISRLE | VL |
| PEDFAVYYCQLYGASSVTFGQGTKLEIK | |
| EIVLTQSPGTLSLSPGERATLSCRASQGISSAYLAWYQQK | STSA-C27-33-33- |
| PGQAPRLLIYGTSRRATGIPDRESGSGSGTDFTLTISRLEP | VL |
| EDFAVYYCQLYGATSVTFGQGTKLEIK | |
| EIVLTQSPGTLSLSPGERATLSCRASQSVSSAYLAWYQQ | STSA-C27-56-56- |
| KPGQAPRLLIYGTSRRATGIPDRFSGSGSGTDFTLTISRLE | VI |
| PEDFAVYYCQLYGASSVTFGQGTKLEIK | |
| EIVLTQSPGTLSLSPGERATLSCRASQSISTAYLAWYQQK | STSA-C27-78-78- |
| PGQAPRLLIYGTSRRATGIPDRESGSGSGTDFTLTISRLEP | VL |
| EDFAVYYCQLYGASSVTFGQGTKLEIK | |
| EIVLTQSPGTLSLSPGERATLSCRASQDISSAYLAWYQQK | STSA-C27-82-58- |
| PGQAPRLLIYGTSRRATGIPDRESGSGSGTDFTLTISRLEP | VL |
| EDFAVYYCQLYGATSVTFGQGTKLEIK | |
| EIVLTQSPGTLSLSPGERATLSCRASQDVSSAYLAWYQQ | STSA-C27-54-54- |
| KPGQAPRLLIYGTSRRATGIPDRFSGSGSGTDFTLTISRLE | VI |
| PEDFAVYYCQLYGATSVTFGQGTKLEIK | |
| EIVLTQSPGTLSLSPGERATLSCRASQNISTAYLAWYQQK | STSA-C27-36-36- |
| PGQAPRLLIYGTSRRATGIPDRESGSGSGTDFTLTISRLEP | VI |
| EDFAVYYCQLYGATSVTFGQGTKLEIK | |
| EIVLTQSPGTLSLSPGERATLSCRASQDASNAYLAWYQQ | STSA-C27-53-53- |
| KPGQAPRLLIYGTSRRATGIPDRFSGSGSGTDFTLTISRLE | VI |
| PEDFAVYYCQLYGSSSVTFGQGTKLEIK | |
| EIVLTQSPGTLSLSPGERATLSCRASQGVSSAYLAWYQQ | STSA-C27-67-67- |
| KPGQAPRLLIYGTSRRATGIPDRFSGSGSGTDFTLTISRLE | VL |
| PEDFAVYYCQLYGRSSVTFGQGTKLEIK | |
| EIVLTQSPGTLSLSPGERATLSCRASQNISTAYLAWYQQK | STSA-C27-55-55- |
| PGQAPRLLIYGTSRRATGIPDRESGSGSGTDFTLTISRLEP | VL |
| EDFAVYYCQLYGTSSVTFGQGTKLEIK | |
| EIVLTQSPGTLSLSPGERATLSCRASQSVSTAYLAWYQQ | STSA-C27-59-59- |
| KPGQAPRLLIYGTSRRATGIPDRFSGSGSGTDFTLTISRLE | VL |
| PEDFAVYYCQLYGATSVTFGQGTKLEIK | |
| EIVLTQSPGTLSLSPGERATLSCRASQDISSAYLAWYQQK | STSA-C27-58-58- |
| PGQAPRLLIYGTSRRATGIPDRESGSGSGTDFTLTISRLEP | VL |
| EDFAVYYCQLYGATSVTFGQGTKLEIK | |
| EIVLTQSPGTLSLSPGERATLSCRASQGVSTAYLAWYQQ | STSA-C27-52-52- |
| KPGQAPRLLIYGTSRRATGIPDRFSGSGSGTDFTLTISRLE | VL |
| PEDFAVYYCQLYGATSVTFGQGTKLEIK | |
| EIVLPQSPGTLSLSPGERATLSCRASQGVSSAYLAWYQQ | STSA-C27-Y2-Y2- |
| KPGQAPRLLIYGTSRRATGIPDRFSGSGSGTDFTLTISRLE | VL |
| PEDFAVYYCQLYGSTSVTFGQGTKLEIK | |

In certain embodiments, an antibody or antigen-binding fragment thereof of the disclosure comprises heavy chain variable region (HCVR) and light chain variable region (LCVR) sequence pairs (HCVR/LCVR) selected from the group consisting of: Y0188-1/Y0188-1; Y0188-2/Y0188-2; Y0188-3/Y0188-3; Y0188-4/Y0188-4; Y0188-6/Y0188-6; Y0188-8/Y0188-8; Y0188-9/Y0188-9; Y0188-10/Y0188-10; Y0188-14/Y0188-14; HV3-15-14/YO1-14; HV3-15-14/164-14; HV3-15-14/KV4-14; HV3-15-14/KV1-27-14; HV3-15-14/KV1-9-14; HV3-15-14/KV1-NL1-14; HV3-15-14/KV1D-43-14; HV3-48-14/YO1-14; HV3-48-14/164-14; HV3-48-14/KV4-14; HV3-48-14/KV1-27-14; HV3-48-14/KV1-9-14; HV3-48-14/KV1-NL1-14; HV3-48-14/KV1D-43-14; HV3-73*2-14/YO1-14; HV3-73*2-14/164-14; HV3-73*2-14/KV4-14; HV3-73*2-14/KV1-27-14; HV3-73*2-14/KV1-9-14; HV3-73*2-14/KV1-NL1-14; HV3-73*2-14/KV1D-43-14; HV3-72-14/YO1-14; HV3-72-14/164-14; HV3-72-14/KV4-14; HV3-72-14/KV1-27-14; HV3-72-14/KV1-9-14; HV3-72-14/KV1-NL1-14; HV3-72-14/KV1D-43-14; YO1-14/YO1-14; YO1-14/164-14; YO1-14/KV4-14; YO1-14/KV1-27-14; YO1-14/KV1-9-14; YO1-14/KV1-NL1-14; YO1-14/KV1D-43-14; 162-14/Y01-14; 162-14/164-14; 162-14/KV4-14; 162-14/KV1-27-14; 162-14/KV1-9-14; 162-14/KV1-NL1-14; 162-14/KV1D-43-1L; VH73-14/Y01-14; VH73-14/164-14; VH73-14/KV4-14; VH73-14/KV1-27-14; VH73-14/KV1-9-14; VH73-14/KV1-NL1-14; and VH73-14/KV1D-43-14.

The antibodies recited below in Table 6 are described in more detail in WO2021/213329, incorporated herein by reference in its entirety for all purposes.

TABLE 6

| | |
|---|---|
| Y0188-1 | EVQLVESGGGLVQPKGSLKLSCAASGFTFNTYGMHWVRQAPG |
| VH | KGLEWVAHIRSKSSNYATYYADSVKDRFTISRDDSQSMLYLQM |
| | NNLKTEDTAMYYCVRWFRAMDYWGQGTSVTVSS |
| Y0188-2 | EVQLIESGGGLVQPKGSLKLSCAASGFTFNMYAMDWVRQAPGK |
| VH | GLEWVARIRSKGSNFETNYADSVKDRFTISRDDSQSMVYLQMIN |
| | LKTEDTAMYYCVRHRGGAWFAYWGQGTLVSVSA |
| Y0188-3 | QVQLVETGGGLVRPGNSLKLSCVTSGFTFSNYRMHWLRQPPGK |
| VH | RLEWIAVITVKSNNYGANYAESVKGRFAISRDDSKSSVYLEMNR |
| | LREEDTATYFCSRERAYGNPFDYWGQGTTLTVSS |
| Y0188-4 | EVQLVESGGGLVQPKGSLKLSCAASGFTFNMYAMNWVRQAPG |
| VH | QGLEWVARIRSKSNNYATYYADSVKDRFIISRDDSESMVYLQMS |
| | NLRAADTAMYYCVRHLRAMDYWGQGTSVTVSS |
| Y0188-6 | EVQLVESGGGLVQPKGSLKLSCAASGFSFNMYAMNWVRQAPG |
| VH | KGLEWVARIRTKSNHYSTYYADSVKDRFTISRDDSASMFYLQM |
| | NNLKTEDTAMYFCVRHLRAMDYWGQGTSVTVSS |
| Y0188-8 | EVQLIESGGGLVQPKGSLKLSCAASGFTFNMYAMDWVRQAPGK |
| VH | GLEWVARIRSKGSNFETNYADSVKDRFTISRDDSQSMVYLQMN |
| | NLKTEDTAMYYCVRHRGGAWFAYWGQGTLVTVSA |
| Y0188-9 | EVQLVESGGGLVRPKGSLKLSCAASGFSFNTYAMNWVRQAPGK |
| VH | GLEWIVWIRSKSHNYATYYADSVKDRFTISRDDSESMLYLQMN |
| | NLKTEDTAMYYCVRHLRAMDYWGQGTSVTVSS |
| Y0188-10 | EVRLVESGGGLVQPKGSLKLSCEASGFSFNMYAMNWVRQAPG |
| VH | KGLEWITHIRSKSNNYATYYADSVKDRFIISRDDSESMVYLQMN |
| | NLKTEDTAMYYCVRLLRALDYWGQGTSVTVSS |
| Y0188-14 | EVQLVESGGGLVQPKGSLKLSCAASGFTFNMYGMHWVRQAPG |
| VH | KGLEWVAHIRSKSSNYATYYADSVKDRLTISRDDSQSMLYLQM |
| | NNLKTEDTAMYYCVRWFRAMDYWGQGTSVTVSS |
| HV3-15-14 | EVQLVESGGGLVKPGGSLRLSCAASGFTFSMYGMHWVRQAPG |
| VH | KGLEWVGHIRSKSSNYATYYADSVKDRFTISRDDSKNTLYLQM |
| | NSLKTEDTAVYYCTTWFRAMDYWGQGTLVTVSS |
| HV3-48-14 | EVQLVESGGGLVQPGGSLRLSCAASGFTFSMYGMHWVRQAPG |
| VH | KGLEWVSHIRSKSSNYATYYADSVKDRFTISRDNAKNSLYLQM |
| | NSLRAEDTAVYYCARWFRAMDYWGQGTLVTVSS |
| HV3-73*2- | EVQLVESGGGLVQPGGSLKLSCAASGFTFSMYGMHWVRQASG |
| 14 | KGLEWVGHIRSKSSNYATYYADSVKDRFTISRDDSKNTAYLQM |
| VH | NSLKTEDTAVYYCTRWFRAMDYWGQGTLVTVSS |
| HV3-72-14 | EVQLVESGGGLVQPGGSLRLSCAASGFTFSMYGMHWVRQAPG |
| VH | KGLEWVGHIRSKSSNYATYYADSVKDRFTISRDDSKNSLYLQM |
| | NSLKTEDTAVYYCARWFRAMDYWGQGTLVTVSS |
| Y01-14 | EVQLVESGGGLVQPGGSLRLSCAASGFTFSMYGMHWVRQAPG |
| VH | KGLEWVSHIRSKSSNYATYYADSVKDRFTISRDNAKNSLYLQM |
| | NSLRAEDTAVYYCARWFRAMDYWGQGTLVTVSS |
| 162-14 | EVQLVESGGGLEQPGGSLRLSCAGSGFTFRMYGMHWVRQAPG |
| VH | KGLEWVSHIRSKSSNYATYYADSVKDRFTISRDNSKNTLYLQM |
| | NSLRAEDTAVYYCAKWFRAMDYWGQGTTVTVSS |
| VH73-14 | EVQLVESGGGLVQPGGSLKLSCAASGFTFSMYGMHWVRQASG |
| VH | KGLEWVGHIRSKSSNYATYYADSVKDRFTISRDDSKNTAYLQM |
| | NSLKTEDTAVYYCTRWFRAMDYWGQGTTVTVSS |
| Y0188-1 | DIVMTQSHKFMSTSVGDRVSITCKASQDVSTAVAWYQEKPGQS |
| VL | PKLLIYWASTRHTGVPDRFTGSGSGTDYTLTISSVQAEDLALYY |
| | CQQHYSTPLTFGAGTKLELK |
| Y0188-2 | DIVVTQSPASLAVSLGQRATISCRASKSVSTSGYSYMHWYQQKP |
| VL | GQPPKLLIYLASNLESGVPARFSGSGSGTDFTLNIHPVEEEDVAIY |
| | YCQHSRELPLTFGAGTKLELK |
| Y0188-3 | DIQMTQSPSSLSASLGERVSLTCRASQEISGYLSWLQQKPDGTIK |
| VL | RLIYAASTLDSGVPKRFSGSRSGSDYSLTISSLESEDFADYYCLQY |
| | GSYPYTFGGGTKLEIK |
| Y0188-4 | DIVLTQSPASLTVSLGQRATISCRASKSVSTSGYSYMHWYQQKP |
| VL | GQPPKLLIYLASNLESGVPARFSGSGSGTDFTLNIHPVEEEDAAT |
| | YYCQHSRELPITFGSGTKLEIK |
| Y0188-6 | DIVLTQSPASLVVSLGQRATISCRASQSVSTSGYSYMHWYQQKP |
| VL | GQPPKLLIYLASNVQSGVPARFSGSGSGTDFTLNIHPVEEEDVAT |
| | YYCHHNRDLPFTFGSGTKLEIK |
| Y0188-8 | DIVVTQSPASLAVSLGQRATISCRASKSVSTSGYSYMHWYQQKP |
| VL | GQPPKLLIYLASNLESGVPARFSGSGSGTDFTLNIHPVEEEDVAIY |
| | YCQHSRELPLTFGAGTKLELK |
| Y0188-9 | DIVLTQSPASLAVSLGQRATISCRASKSVSASGYSYMHWYQQKP |
| VL | GQPPKLLIYLASNLQSGVPARFSGSGSGTDFTLNIHPVEEEDAAT |
| | YYCQHSRELPPTFGGGTKLEIK |
| Y0188-10 | DIVLTQSPASLAVFLGQRATISCRASKSVSTSGYSYMHWYQQKA |
| VL | GQPPKLLIYLASNLESGVPARFSGSGSGTDFTLNIHPVEEEDAAT |
| | YYCHHSRELPITFGSGTKLEMK |
| Y0188-14 | DIVMTQSHKFMSTSVGDRVSITCKASQDVSTAVAWYQEKPGQS |
| VL | PKLLIYWASTRHTGVPDRFTGSGSGTDYTLTISSVQAEDLALYY |
| | CQQHYSTPLTFGAGTKLELK |
| Y01-14 | EIVLTQSPGTLSLSPGERATLSCKASQDVSTAVAWYQQKPGQAP |
| VL | RLLIYWASTRHTGIPDRFSGSGSGTDFTLTISRLEPEDFAVYYCQ |
| | QHYSTPLTFGQGTKVEIK |
| 164-14 | DIVMTQSPLSLPVTPGEPASISCKASQDVSTAVAWYLQKSGQSP |
| VL | QLLIYWASTRHTGVPDRFSGSGSGTDFTLKISRVEAEDVGFYYC |
| | QQHYSTPLTFGQGTKLEIK |
| KV4-14 | DIVMTQSPDSLAVSLGERATINCKASQDVSTAVAWYQQKPGQP |
| VL | PKLLIYWASTRHTGVPDRFSGSGSGTDFTLTISSLQAEDVAVYYC |
| | QQHYSTPLTFGGGTKVEIK |
| KV1-27-14 | DIQMTQSPSSLSASVGDRVTITCKASQDVSTAVAWYQQKPGKVP |
| VL | KLLIYWASTRHTGVPSRFSGSGSGTDFTLTISSLQPEDVATYYCQ |
| | QHYSTPLTFGGGTKVEIK |
| KV1-9-14 | DIQLTQSPSFLSASVGDRVTITCKASQDVSTAVAWYQQKPGKAP |
| VL | KLLIYWASTRHTGVPSRFSGSGSGTEFTLTISSLQPEDFATYYCQ |
| | QHYSTPLTFGGGTKVEIK |
| KV1-NL1-14 | DIQMTQSPSSLSASVGDRVTITCKASQDVSTAVAWYQQKPGKAP |
| VL | KLLLYWASTRHTGVPSRFSGSGSGTDYTLTISSLQPEDFATYYCQ |
| | QHYSTPLTFGGGTKVEIK |
| KV1D-43-14 | AIRMTQSPFSLSASVGDRVTITCKASQDVSTAVAWYQQKPAKAP |
| VL | KLFIYWASTRHTGVPSRFSGSGSGTDYTLTISSLQPEDFATYYCQ |
| | QHYSTPLTFGGGTKVEIK |

The antibodies recited below in Table 7 are described in more detail in U.S. Pat. No. 11,725,057 B2, incorporated herein by reference in its entirety for all purposes.

TABLE 7

| | |
|---|---|
| 1A6 | QVQLQQSGTELVKPGASVKMSCKASVNTFTGYNMHWIKQTP |
| VH | GQGLEWIGGLHPGNGDSSYNQKFKGRATLTDKSSNTAYMQL |
| | SSLTSEDSAVYYCALTTAGRAWFPYWGQGTLVTVSA |
| 1A6 | DIQMTQTTSSLSASLGDRVTISCRASQDISNYLNWYQQKPD |
| VL | GTVKLLIYYTSRLHSGVPSRFSGSGSGTDYSLTISNLEQED |
| | IATYFCQQGNTLPPTFGGGTKLEIK |
| 1D8 | QVQLQQSGTELVKPGASVKMSCKASVNTFTGYNMHWIKQTP |
| VH | GQGLEWIGGLHPGNGDSSYNQKFKGRATLTADKSSNTAYMQ |
| | LSSLTSEDSAVYYCALTTAGRAWFAYWGQGTLVTVSA |
| 1D8 | DIQMTQTTSSLSASLGDRVTISCRASQDISNYLNWYQQKPD |
| VL | GTVKLLIYYTSRLHSGVPSRFSGSGSGTDYSLTISNLEQED |
| | IATYFCQQGNTLHTFGGGTKLEIK |
| 1H9 | QVQLQQSGAELVKPGASVKMSCKASVNTFAGYNMHWVKQTP |
| VH | GQGLEWIGGLHPGNGDTSYNQKFKGKATLTADKSSNTAYMQ |
| | LSSLTSEDSAVYYCALTTAGRAWFAYWGQGTLVTVSA |
| 1H9 | DIQMIQSTSSLSASLGDRVTISCRASQDISNYLNWYQQKPD |
| VL | GTVKLLIYYTSRLHSGVPSRFSGSGSGTDYSLTISNLEQED |
| | IATYFCQQGNTLPWTFGGGTKLEIK |
| 2H1 | QVQLQQSGAELVKPGASVKMSCKASVNTFTGYNMHWVKQTP |
| VH | GQGLEWIGGLHPGNGDTSYNQKFKGKATLTADKSSNTAYMQ |
| | LSSLTSEDSAVYYCALTTAGRAWFAYWGQGTLVTVSA |
| 2H1 | DIQMIQSTSSLSASLGDRVTISCRASQDISNYLNWYQQKPD |
| VL | GTVKLLIYYTSRLHSGVPSRFSGSGSGTDYSLTISNLEQED |
| | IATYFCQQGNTLPLTFGAGTKLELK |
| 2F8 | QVQLQQSGAELVKPGASVKMSCKASVNTFTGYNMHWVKQTP |
| VH | GQGLEWIGGLHPGNGDTSYNQKFKGKATLTADKSSNTAYMQ |
| | LSSLTSEDSAVYYCALTTAGRAWFPYWGQGTLVTVSA |
| 2F8 | DIQMIQSTSSLSASLGDRVTISCRASQDISNYLNWYQQKPD |
| VL | GTVKLLIYYTSRLHSGVPSRFSGSGSGTDYSLTISNLEQED |
| | IATYFCQQGNTLPWTFGGGTKLELK |
| 9B4 | QVQLQQSGAELVKPGASVKMSCKASVNTFTGYNMHWIKQTP |
| VH | GQGLEWIGGLHPGNGDTSYNQKFKGKATLTADKSSNTAYMQ |
| | LSSLTSEDSAVYYCALTTAGRAWFAYWGQGTLVTVSA |
| 9B4 | DIQMIQSTSSLSASLGDRVTISCRASQDISNYLNWYQQKPD |
| VL | GTVKLLIYYTSRLHSGVPSRFSGSGSGTDYSLTISNLEQED |
| | IATYFCQQGNTLPWTFGGGTKLELK |
| 9E7 | EVQLQQSGAELVKPGASVKMSCKASVNIFTGYNMHWVKQTP |
| VH | GQGLEWIGGLHPGNGDTSYNQKFKDKATLTADKSSNTAYMQ |
| | LSSLTSEDSAVYYCALTTAGRAWFAYWGQGTLVTVSA |
| 9E7 | DIQMTQTTSSLSASLGDRVTISCRASQDISNYLNWYQQKPD |
| VL | GTVKLLIYYTSRLHSGVPSRFSGSGSGTDYSLTISNLEQED |
| | IATYFCQQGNTLPWTFGGGTKLELK |
| 24G10 | EVKLVESGGGLVKPGGSLKLSCAASGFTFSRYAMSWVRQTP |
| VH | EKRLEWVATISSGGSYTNYADSVKGRFTISRDNVKNTLYLQ |
| | MNSLRAEDTAVYYCARATARATEFAYWGQGTLVTVSS |
| 24G10 | DIQMTQTTSSLSASLGDRVTISCRASQDISNYLNWYQQKPD |
| VL | GTVKLLIYYTSRLHSGVPSRFSGSGSGTDYSLTISNLEQED |
| | IATYFCQQGNTLPPTFGGGTKLEL |
| 25D6 | EVQLQQSGAELVKPGASVKMSCKASVNIFTGYNMHWVKQTP |
| VH | GQGLEWIGGLHPGNGDTSYNQKFKDKATLTADKSSNTAYMQ |
| | LSSLTSEDSAVYYCALTTAGRAWFAYWGQGTLVTVSA |
| 25D6 | DIQMTQSTSSLSASLGDRVTISCRASQDISNYLNWYQQKPD |
| VL | GTVKLLIYYTSRLHSGVPSRFSGSGSGTDYSLTISNLEQED |
| | IATYFCQQGNTLPWTFGGGTKLEIK |
| 25G9 | EVQLKESGAELVKPGASVKMSCKASVNIFTGYNMHWVKQTP |
| VH | GQGLEWIGGLHPGNGDTSYNQKFKDKATLTADRSSNTAYMQ |
| | LSSLTSEDSAVYYCALTTAGRAWFAYWGQGTLVTVSA |
| 25G9 | DIQMTQSTSSLSASLGDRVTISCRASQDISNYLNWYQQKPD |
| VL | GTVKLLIYYTSRLHSGVPSRFSGSGSGTDYSLTISNLEQED |
| | IATYFCQQGNTLPWTFGGGTKLEIK |
| 35A7-1 | QVQLKQSGAELVKPGASVKMSCTASVNIFTGYNMHWIKQTP |
| VH | GQGLEWIGGLHPGNGDTSYNQKFKDKATLTADRSSNTAYMQ |
| | LSSLTSEDSAVYYCALTTAGRAWFAYWGQGTLVTVSA |
| 35A7-1 | DIQMNQSTSSLSASLGDRVTISCRASQDISNYLNWYQQKPD |
| VL | GTIKLLIYYTSRLYSGVPSRFSGSGSGTDYSLTISNLEEED |
| | IATYFCQQGNTIPYTFGGGTKLEIK |
| 31B9 | EVQLQQSGAELVKPGASVKMSCKASVNIFTGYNMHWVKQTP |
| VH | GQGLEWIGGLHPGNGDTSYNQKFKDKATLTADRSSNTAYMQ |
| | LSSLTSEDSAVYYCALTTAGRAWFAYWGQGTLVTVSA |
| 31B9 | DIQMTQSTSSLSASLGDRVTISCRASQDISNYLNWYQQKPD |
| VL | GTIKLLIYYTSRLHSGVPSRFSGSGSGTDYSLTISNLEEED |
| | IATYFCQQGNTLPWTFGGGTKLEIK |
| 34A2 | EVQLQQSGAELVKPGASVKMSCKASVNIFTSYNMHWVKQTP |
| VH | GQGLEWIGGLHPGNGDTSYNQKFKGKATLTADKSSSTAYMQ |
| | VSMLTSEDSAVYYCVLTTAGRAWFAYWGQGTLVTVSA |
| 34A2 | DIQMTQSTSSLSASLGDRVTISCRASQDISNYLNWYQQKPD |
| VL | GTIKLLIYYTSRLHSGVPSRFSGSGSGTDYSLTISNLEEED |
| | IATYFCQQGNTLPWTFGGGTKLEIK |
| 34H11 | QVQLQESGAELVKPGASVKMSCKASVNTFTGYNMHWVKQTP |
| VH | GQGLEWIGGLHPGNGDTSYNQKFKGKATLTADRSSNTAYMQ |
| | LSSLTSEDSAVYYCALTTAGRAWFAYWGQGTLVTVSA |
| 34H11 | DIQMTQTTSSLSASLGDRVTISCRASQDISNYLNWYQQKPD |
| VL | GTVKLLIYYTSRLHSGVPSRFSGSGSGTDYSLTISNLEEED |
| | IATYFCQQGNTLPWTFGGGTKLEIK |
| 35D5 | QVQLKQSGAELVKPGASVKMSCKASVNTFTGYNMHWVKQTP |
| VH | GQGLEWIGGLHPGNGDTSYNQKFKGKATLTADKSSNTAYMQ |
| | LSSLTSEDSAVYYCALTTAGRAWFAYWGQGTLVTVSA |
| 35D5 | DIQMTQSTSSLSASLGDRVTISCRASQDISNYLNWYQQKPD |
| VL | GTVKLLIYYTSRLHSGVPSRFSGSGSGTDYSLTISNLEEED |
| | IATYFCQQGNTLPWTFGGGTKLEIK |
| 35A7-2 | QVQLKQSGAELVKPGASVKMSCKASVNTFTGYNMHWIKQTP |
| VH | GQGLEWIGGLHPGNGDTSYNQKFKGKATLTADKSSNTAYMQ |
| | LSSLTSEDSAVYYCALTTAGRAWFAYWGQGTLVTVSA |
| 35A7-2 | DIQMNQSTSSLSASLGDRVTISCRASQDISNYLNWYQQKPD |
| VL | GTIKLLIYYTSRLYSGVPSRFSGSGSGTDYSLTISNLEEED |
| | IATYFCQQGNTLPYTFGGGTKLEIK |
| 36F4 | QVQLQQSGAELVKPGASVKMSCKASVNTITGYNMHWVKQTP |
| VH | GQGLEWIGGLHPGNGDTSYNQKFKDKATLTADKSSNTAYMQ |
| | LSSLTSEDSAVYYCALTTAGRAWFAYWGQGTLVTVSA |
| 36F4 | DIKMTQSTSSLSASLGDRVTTSCRASQDISNYLNWYQQKPD |
| VL | GTVKLLIYYTSRLHSGVPSRFSGSGSGTDYSLTISNLEEED |
| | IATYFCQQGNTLPPWTFGGGTKLEIK |
| VH1021 | EVQLVESGGGLVKPGGSLRLSCAASGFTFSRYAMSWVRQAP |
| | GKGLEWVSTISSGGSYTNYADSVKGRFTISRDNVKNTLYLQ |
| | MNSLRAEDTAVYYCARATARATEFAYWGQGTLVTVSS |
| VH1022 | EVQLLESGGGLVQPGGSLRLSCAASGFTFSRYAMSWVRQAP |
| | GKGLEWVSTISSGGSYTNYADSVKGRFTISRDNVKNTLYLQ |
| | MNSLRAEDTAVYYCARATARATEFAYWGQGTLVTVSS |
| VH1023 | QVQLVQSGAELKKPGASVKVSCKASGNTFTGYNMHWIQQSP |
| | GQGLEWMGGLHPGNGDSSYNQKFQGRVTLTADKSSNTAYME |
| | LSSLRSEDSAVYYCALTTAGRAWFPYWGQGTLVTVSS |
| VH1024 | QVQLVQSGAEVKKPGASVKMSCKASVYTFTGYNMHWIQQSP |
| | GQGLEWMGGLHPGNGDSSYNQKFQGRATLTADKSSNTAYME |
| | LSSLRSEDSAVYYCALTTAGRAWFPYWGQGTLVTVSS |
| VH1025 | QVQLVQSGAEVKKPGASVKVSCKASGNIFTGYNMHWVRQAP |
| | GQGLEWMGGLHPGNGDSSYNQKFQGRVTLTADKSSNTAYME |
| | LSSLRSEDSAVYYCALTTAGRAWFAYWGQGTLVTVSS |
| VH1026 | QVQLVQSGAEVKKPGASVKMSCKASVYTFTGYNMHWVRQAP |
| | GQGLEWIGGLHPGNGDSSYNQKFQGRATLTADKSSNTAYME |
| | LSSLRSEDSAVYYCALTTAGRAWFAYWGQGTLVTVSS |
| VH1027 | EVQLVQSGAEVKKPGATVKISCKASGNIFTSYNMHWVRQAP |
| | GQGLEWMGGLHPGNGDTSYNQKFQGRVTLTADKSSSTAYME |
| | LSSLRSEDTAVYYCVLTTAGRAWFAYWGQGTLVTVSS |
| VH1028 | EVQLVQSGAEVKKPGATVKMSCKASVYTFTSYNMHWVRQAP |
| | GQGLEWIGGLHPGNGDTSYNQKFKGRATLTADKSSSTAYME |
| | LSSLRSEDTAVYYCVLTTAGRAWFAYWGQGTLVTVSS |
| VL1011 | DIQMTQSPSSLSASVGDRVTITCQASQDISNYLNWYQQKPG |
| | KAPKLLIYYTSRLHSGVPSRFSGSGSGTDYTFTISSLQPED |
| | IATYFCQQGNTLPWTFGGGTKVEIK |
| VL1012 | DIQMTQSPSSLSASVGDRVTITCRASQDISNYLNWYQQKPG |
| | KAPKLLIYYTSRLHSGVPSRFSGSGSGTDYTLTISSLQPED |
| | FATYFCQQGNTLPWTFGGGTKVEIK |
| VL1013 | DIQMTQSPSSLSASVGDRVTITCQASQDISNYLNWYQQKPG |
| | KAPKLLIYYTSRLHSGVPSRFSGSGSGTDYTLTISSLQPED |
| | FATYFCQQGNTLPLTFGGGTKVEIK |
| VL1014 | DIQMTQSPSSLSASVGDRVTITCQASQDISNYLNWYQQKPG |
| | KAPKLLIYYTSRLHSGVPSRFSGSGSGTDYTLTISSLQPED |
| | FATYFCQQGNTLPWTFGGGTKVEIK |

The antibodies recited below in Table 8 are described in more detail in EP 3904385 A1 and US 2023/0295312, incorporated herein by reference in their entireties for all purposes.

TABLE 8

| | |
|---|---|
| AK-9 | GYTFTEYT |
| AK-10 | INPNNGGT |
| AK-11 | ARVRRGMDY |
| AK-12 | QDVTTA |
| AK-13 | SAS |
| AK-14 | QQHYSAPWT |
| AK-15 | GFTFSSSY |
| AK-16 | INSNGGKT |
| AK-17 | TRQRGNYVGAMDY |
| AK-18 | QDVSTA |
| AK-19 | SAS |
| AK-20 | HQYYGSPPT |
| AK-130 | GFSLSTSGMG |
| AK-131 | IWWADDK |
| AK-132 | ARITRGNSAMDF |
| AK-133 | ENVYSY |
| AK-134 | NAK |
| AK-135 | QHHYGIPWT |
| AK-2 | EVQLQQSGPELVKPGASVKISCKTSGYTFTEYTIH |
| | WVKQNHGKSLEWIGGINPNNGGTVYNQNFKGKA |
| | TLTVDKSSSTAYMELRSLTSEDSAVYYCARVRRGM |
| | DYWGQGTSVTVSS |
| AK-4 | DIVMTQSHKFMSTSVGDRVSITCKASQDVTTAVAW |
| | YQQKPGQSPKLLIYSASYRYTGVPDRFTGSGSGTDF |
| | TFTISSVQAEDLAVYYCQQHYSAPWTFGGGTNLEIK |
| AK-22 | QVQLQQSGAEVVKPGASVKISCKTSGYTFTEYTIHW |
| | VKQAHGQSLEWIGGINPNNGGTVYNQKFQGKATLT |
| | VDKSTSTAYMELRSLTSEDTAVYYCARVRRGMDYW |
| | GQGTSVTVSS |
| AK-24 | DIQMTQSPKSLSTSVGDRVTITCRASQDVTTAVAW |
| | YQQKPGKSPKLLIYSASYRYTGVPSRFSGSGSGTDF |
| | TFTISSVQPEDLATYYCQQHYSAPWTFGGGTNLEIK |
| AK-VH 34 | QVQLVQSGAEVVKPGASVKVSCKTSGYTFTEYT |
| | IHWVRQAPGQSLEWIGGINPNNGGTVYNQKFQG |
| | KVTLTVDKSTSTAYMELSSLRSEDTAVYYCARV |
| | RRGMDYWGQGTSVTVSS |
| AK-VL 36 | DIQMTQSPSSLSASVGDRVTITCRASQDVTTAVA |
| | WYQQKPGKAPKLLIYSASYRYTGVPSRFSGSGSG |
| | TDFTLTISSVQPEDLATYYCQQHYSAPWTFGGGT |
| | NLEIK |

In certain embodiments, an antibody or antigen-binding fragment thereof of the disclosure comprises heavy chain CDR and light chain CDR sequence pairs (SN-HC CDRs/SN-LC CDRs) of: SN-1, SN-2, SN-3/SN-4, SN-5, SN-6; or SN-14, SN-15, SN-16/SN-17, SN-18, SN-19.

In certain embodiments, an antibody or antigen-binding fragment thereof of the disclosure comprises an SN-HCVR/SN-LCVR sequence pair of: SN-7/SN-8; SN-12/SN-13; or SN-VH 12/SN-VL 13.

In certain embodiments, an antibody or antigen-binding fragment thereof of the disclosure comprises a SN-HCVR/SN-LCVR sequence pair of SN-VH 12/SN-VL 13.

In certain embodiments, an antibody or antigen-binding fragment thereof of the disclosure comprises the HCVR/LCVR pair of anti-IL-4Rα monoclonal antibody SNC005 (also referred to as QXHG5N and PD2-31).

The antibodies recited below in Table 9 are described in more detail in US 2022/0073631, incorporated herein by reference in its entirety for all purposes.

TABLE 9

| | |
|---|---|
| SN-1 | TNSMS |
| SN-2 | IIGSSGYMDYASWAKG |
| SN-3 | HGDSSSFAL |
| SN-4 | RASESVYKNNRLS |
| SN-5 | EASKVAS |
| SN-6 | AGAYRGNIYP |
| SN-14 | SNAVG |
| SN-15 | FINFSGITYYANWAKG |
| SN-16 | GSAGSFDL |
| SN-17 | QASESVYKNNYLA |
| SN-18 | RASNLAS |
| SN-19 | QGGYSSGMYP |
| SN-7 | EVQLVESGGGLVQPGGSLRLSCAASGIDLRTNSMSW |
| | VRQAPGKGLEWVGIIGSSGYMDYASWAKGRFTISRD |
| | NSKNTLYLQMNSLRAEDTAVYYCARHGDSSSFALW |
| | GQGTLVTVSS |
| SN-8 | DIQMTQSPSSLSASVGDRVTITCRASESVYKNNRLSW |
| | YQQKPGKAPKLLIYEASKVASGVPSRFSGSGSGTDFT |
| | LTISSLQPEDFATYYCAGAYRGNIYPFGQGTKVEIK |
| SN-12 | EVQLVESGGGLVQPGGSLRLSCAASGFSLNSNAVGW |
| | VRQAPGKGLEYIGFINFSGITYYANWAKGRFTISRD |
| | NSKNTLYLQMNSLRAEDTAVYYCARGSAGSFDLW |
| | GQGTLVTVSS |
| SN-13 | DIQMTQSPSSLSASVGDRVTITCQASESVYKNNYLAW |
| | YQQKPGKAPKLLIYRASNLASGVPSRFSGSGSGTDFTL |
| | TISSLQPEDFATYYCQGGYSSGMYPFGQGTKVEIK |
| SN-VH 12 | EVQLVESGGGLVQPGGSLRLSCAASGFSLNSNAVGW |
| | VRQAPGKGLEYIGFINFSGITYYANWAKGRFTISRDN |
| | SKNTLYLQMNSLRAEDTAVYYCARGSAGSFDLWGQ |
| | GTLVTVSS |
| SN-VH 13 | DIQMTQSPSSLSASVGDRVTITCQASESVYKNNYLAW |
| | YQQKPGKAPKLLIYRASNLASGVPSRFSGSGSGTDFT |
| | LTISSLQPEDFATYYCQGGYSSGMYPFGQGTKVEIK |

In certain embodiments, an antibody or antigen-binding fragment thereof of the disclosure comprises heavy chain CDR and light chain CDR sequence pairs (SHR-HC CDRs/SHR-LC CDRs) of: SHR-3, SHR-4, SHR-5/SHR-6, SHR-7, SHR-8; SHR-3, SHR-4, SHR-5/SHR-38, SHR-7, SHR-40; or SHR-3, SHR-4, SHR-5/SHR-42, SHR-39, SHR-8.

In certain embodiments, an antibody or antigen-binding fragment thereof of the disclosure comprises an SHR-HCVR/SHR-LCVR sequence pair of: SHR-1/SHR-2; SHR-43/SHR-37; SHR-43/SHR-41; or SHR-VH 25/SHR-VL 34.

In certain embodiments, an antibody or antigen-binding fragment thereof of the disclosure comprises a SHR-HCVR/SHR-LCVR sequence pair of SHR-VH 25/SHR-VL 34.

In certain embodiments, an antibody or antigen-binding fragment thereof of the disclosure comprises the HCVR/LCVR pair of anti-IL-4Rα monoclonal antibody SHR-1819.

The antibodies recited below in Table 10 are described in more detail in US 2020238294, incorporated herein by reference in its entirety for all purposes.

TABLE 10

| | |
|---|---|
| SHR-3 | GFTFSDYGMH |
| SHR-4 | FISSGSSIIYYADIVK |
| SHR-5 | GNKRGFFDY |
| SHR-6 | NASSSVSYMY |
| SHR-7 | LTSNLAS |
| SHR-8 | QQWRSNPPMLT |
| SHR-38 | RASSSVPYMY |
| SHR-40 | QQWRAYPPMLT |
| SHR-42 | RASPGVPPLA |
| SHR-39 | LASSRPS |
| SHR-1 | EVQLVESGGGLVKPGGSLKLSCAASGFTFSDYGMHW |
| | VRQAPEKGLEWVAFISSGSSIIYYADIVKGRSTISR |
| | DNAKNTLFLQMTSLRSEDTAMYYCTRGNKRGFFDYW |
| | GQGTILTVSS |
| SHR-2 | QIVLTQSPALMSASPGEKVTMTCNASSSVSYMYWYQ |
| | RKPRSSPKPWIYLTSNLASGVPVRFSGSGSGTSYSL |
| | TISSMEAEDAATYYCQQWRSNPPMLTFGSGTKLEVK |
| SHR-43 | EVQLVESGGGLVQPGGSLRLSCAASGFTFSDYGMHW |
| | VRQAPGKGLEWVAFISSGSSIIYYADIVKGRSTISR |
| | DNAKNTLYLQMNSLRAEDTAVYYCTRGNKRGFFDYW |
| | GQGTLVTVSS |
| SHR-37 | EIVLTQSPATLSLSPGERATLSCRASSSVPYMYWYQ |
| | QKPGQAPRLLIYLTSNLASGIPARFSGSGSGTDFTL |
| | TISSLEPEDFAVYYCQQWRAYPPMLTFGGGTKVEIK |
| SHR-VL 41 | EIVLTQSPATLSLSPGERATLSCRASPGVPPLAWYQ |
| | QKPGQAPRLLIYLASSRPSGIPARFSGSGSGTDFTL |
| | TISSLEPEDFAVYYCQQWRSNPPMLTFGGGTKVEIK |
| SHR-VH 25 | EVQLVESGGGLVQPGGSLRLSCAASGFTFSDYGMHW |
| | VRQAPGKGLEWVAFISSGSSIIYYADIVKGRFTISR |
| | DNAKNSLYLQMNSLRAEDTAVYYCARGNKRGFFDYW |
| | GQGTLVTVSS |
| SHR-VL 34 | DIVMTQTPLSLSVTPGQPASISCKASQSVDYGGDSY |
| | MNWYLQKPGQPPQLLIYAASNLESGVPDRFSGSGSG |
| | TDFTLKISRVEAEDVGVYYCQHSNENPPTFGGGTKV |
| | EIK |

In certain embodiments, an antibody or antigen-binding fragment thereof of the disclosure comprises heavy chain CDR and light chain CDR sequence pairs (MG-HC CDRs/MG-LC CDRs) of: MG-129, MG-230, MG-131/MG-132, MG-133, MG-134 (or MG-134 with an L8F mutation).

In certain embodiments, an antibody or antigen-binding fragment thereof of the disclosure comprises an MG-HCVR/MG-LCVR sequence pair of: MG-18/MG-20; or MG-VH 88/MG-VH 91.

In certain embodiments, an antibody or antigen-binding fragment thereof of the disclosure comprises an MG-HCVR/MG-LCVR sequence pair of MG-VH 88/MG-VH 91.

In certain embodiments, an antibody or antigen-binding fragment thereof of the disclosure comprises an MG heavy chain (HC)/MG light chain (LC) pair of MG-89/MG-92.

In certain embodiments, an antibody or antigen-binding fragment thereof of the disclosure comprises the HCVR/LCVR pair of anti-IL-4Rα monoclonal antibody MG-K10.

The antibodies recited below in Table 11 are described in more detail in U.S. Pat. No. 11,939,387 and EP4137517, incorporated herein by reference in their entireties for all purposes.

TABLE 11

| | |
|---|---|
| MG-129 | DYGMH |
| MG-130 | YISSGSTTIYYADTVKG |
| MG-131 | ARISTVVAKRYAMDY |
| MG-132 | RASQDISNYLN |
| MG-133 | YTSRLHS |
| MG-134 | QQINALPLT |
| MG-18 | EVQLVESGGGLVKPGGSLKLSCAASGFTFSDYGMHW |
| | VRQAPEKGLEWVAYISSGSTTIYYADTVKGRFTISRDN |
| | GKNTLFLQMTSLRSEDTAMYYCARISTVVAKRYAMD |
| | YWGQGTSVTVSS |
| MG-20 | DIQMTQTASSLSASLGDRVTISCRASQDISNYLNWYQQ |
| | KPDGTVKLLIYYTSRLHSGVPSRFSGSGSGTDYSLTISN |
| | LEQEDIATYFCQQINALPLTFGAGTKLELK |
| MG-89 | EVQLVESGGGLVQPGGSLRLSCAASGFTFSDYGMHW |
| | VRQAPGKGLEWVSYISSGSTTIYYADTVKGRFTISRDN |
| | AKNSLYLQMNSLRAEDTAVYYCARISTVVAKRYAMD |
| | YWGQGTLVTVSSASTKGPSVFPLAPCSRSTSESTAALG |
| | CLVKDYFPEPVTVSWNSGALTSGVHTFPAVLQSSG |
| | LYSLSSVVTVPSSSLGTKTYTCNVDHKPSNTKVDK |
| | RVESKYGPPCPPCPAPEFLGGPSVFLFPPKPKDTLM |
| | ISRTPEVTCVVVDVSQEDPEVQFNWYVDGVEVHN |
| | AKTKPREEQFNSTYRVVSVLTVLHQDWLNGKEYK |
| | CKVSNKGLPSSIEKTISKAKGQPREPQVYTLPPSQEE |
| | MTKNQVSLTCLVKGFYPSDIAVEWESNGQPENNY |
| | KTTPPVLDSDGSFFLYSRLTVDKSRWQEGNVFSCSV |
| | LHEALHSHYTQKSLSLSLG |
| MG-92 | DIQMTQSPSSLSASVGDRVTITCRASQDISNYLNWY |
| | QQKPGKAPKLLIYYTSRLHSGVPSRFSGSGSGTDFT |
| | LTISSLQPEDIATYYCQQINALPFTFGQGTKLEIKRTV |
| | AAPSVFIFPPSDEQLKSGTASVVCLLNNFYPREAKV |
| | QWKVDNALQSGNSQESVTEQDSKDSTYSLSSTLTLS |
| | KADYEKHKVYACEVTHQGLSSPVTKSFNRGEC |
| MG-VH 88 | EVQLVESGGGLVQPGGSLRLSCAASGFTFSDYGMHW |
| | VRQAPGKGLEWVSYISSGSTTIYYADTVKGRFTISRDN |
| | AKNSLYLQMNSLRAEDTAVYYCARISTVVAKRYAMD |
| | YWGQGTLVTVSS |
| MG-VL 91 | DIQMTQSPSSLSASVGDRVTITCRASQDISNYLNWYQQ |
| | KPGKAPKLLIYYTSRLHSGVPSRFSGSGSGTDFTLTISS |
| | LQPEDIATYYCQQINALPFTFGQGTKLEIK |

In certain embodiments, an antibody or antigen-binding fragment thereof of the disclosure comprises heavy chain CDR and light chain CDR sequence pairs (GR-HC CDRs/GR-LC CDRs) of: GR-33, GR-34, GR-35/GR-36, GR-37, GR-38; GR-33, GR-34, GR-35/GR-39, GR-40, GR-41; GR-33, GR-34, GR-35/GR-42, GR-43, GR-41; GR-33, GR-34, GR-35/GR-42, GR-40, GR-44; GR-33, GR-34, GR-35/GR-45, GR-46, GR-47; GR-33, GR-34, GR-35/GR-48, GR-37, GR-49; GR-VH 18/SHR-VL 26; or GR-VH 18/GR-VL 30.

In certain embodiments, an antibody or antigen-binding fragment thereof of the disclosure comprises an GR-HCVR/GR-LCVR sequence pair of: GR-18/GR-26; GR-18/GR-27; GR-18/GR-28; GR-18/GR-29; GR-18/GR-30; GR-18/GR-31; GR-VH 18/SHR-VL 26; or GR-VH 18/GR-VL 30.

In certain embodiments, an antibody or antigen-binding fragment thereof of the disclosure comprises an GR-HCVR/GR-LCVR sequence pair of: GR-VH 18/SHR-VL 26; or GR-VH 18/GR-VL 30.

In certain embodiments, an antibody or antigen-binding fragment thereof of the disclosure comprises the GR-HCVR/GR-LCVR pair of anti-IL-4Rα monoclonal antibody GR-1802.

The antibodies recited below in Table 12 are described in more detail in U.S. Pat. No. 11,897,960, incorporated herein by reference in its entirety for all purposes.

TABLE 12

| | |
|---|---|
| GR-33 | GFTFSSYAMS |
| GR-34 | SITGGGGGIYYADSVKG |
| GR-35 | DRISITIRPRYFGLDF |
| GR-36 | RSSQSLLYSIGYNYLD |
| GR-37 | LGSNRAS |
| GR-38 | MQSFKAPYT |
| GR-39 | RSSRNVIYGNGYNYLD |
| GR-40 | LGNNVAA |
| GR-41 | MQSLQAPYT |
| GR-42 | RSSQNVYGNGYNYLD |
| GR-43 | LGTNVAA |
| GR-44 | MQSLKAPYT |
| GR-45 | RSSHNLLYSNGYNYLD |
| GR-46 | LGSNRAY |
| GR-47 | MQALQSPYT |
| GR-48 | RSSQSLLYSNGYNYLD |
| GR-49 | MQALETPYA |
| GR-18 | EVQLVESGGGLVQPGGSLRLSCAVSGFTFSSYAMSW |
| | VRQAPGKGLEWVSSITGGGGGIYYADSVKGRFTISRD |
| | NSKNTVYLQMNSLRAEDTAVYYCAKDRISITIRPRYF |
| | GLDFWGQGTTVTVSS |
| GR-26 | DIVMTQSPLSLPVTPGEPASISCRSSQSLLYSIGYNYLD |
| | WYLQKSGQSPQLLIYLGSNRASGVPDRFSGSGSGTDF |
| | TLKISRVEAEDVGFYYCMQSFKAPYTFGQGTKLEIK |
| GR-27 | DIVMTQSPLSLPVTPGEPASISCRSSRNVIYGNGYNYLD |
| | WYLQKSGQSPQLLIYLGNNVAAGVPDRFSGSGSGTDF |
| | TLKISRVEAEDVGFYYCMQSLQAPYTFGQGTKLEIK |
| GR-28 | DIVMTQSPLSLPVTPGEPASISCRSSQNVVYGNGYNYLD |
| | WYLQKSGQSPQLLIYLGTNVAAGVPDRFSGSGSGTDF |
| | TLKISRVEAEDVGFYYCMQSLQAPYTFGQGTKLEIK |
| GR-29 | DIVMTQSPLSLPVTPGEPASISCRSSQNVVYGNGYNYLD |
| | WYLQKSGQSPQLLIYLGNNVAAGVPDRFSGSGSGTDF |
| | TLKISRVEAEDVGFYYCMQSLKAPYTFGQGTKLEIK |
| GR-30 | DIVMTQSPLSLPVTPGEPASISCRSSHNLLYSNGYNYLD |
| | WYLQKPGQSPQLLIYLGSNRAYGVPDRFSGSGSGTDF |
| | TLKISRVEAEDVGVYHCMQALQSPYTFGQGTKVDIK |
| GR-31 | DIVMTQSPLSLPVTAGAPASISCRSSQSLLYSNGYNYLD |
| | WYMQKPGQSPQLLIYLGSNRASGVPDRFSGSGSGTDF |
| | TLKISRVEAEDVGVYYCMQALETPYAFGQGTKVEIK |
| GR-VH 18 | EVQLVESGGGLVQPGGSLRLSCAVSGFTFSSYAMSW |
| | VRQAPGKGLEWVSSITGGGGGIYYADSVKGRFTISRD |
| | NSKNTVYLQMNSLRAEDTAVYYCAKDRISITIRPRYF |
| | GLDFWGQGTTVTVSS |
| GR-VL 26 | DIVMTQSPLSLPVTPGEPASISCRSSQSLLYSIGYNYLD |
| | WYLQKSGQSPQLLIYLGSNRASGVPDRFSGSGSGTDF |
| | TLKISRVEAEDVGFYYCMQSFKAPYTFGQGTKLEIK |
| GR-VL 30 | DIVMTQSPLSLPVTPGEPASISCRSSHNLLYSNGYNY |
| | LDWYLQKPGQSPQLLIYLGSNRAYGVPDRFSGSGSG |
| | TDFTLKISRVEAEDVGVYHCMQALQSPYTFGQGTKVDIK |

In certain embodiments, an antibody or antigen-binding fragment thereof of the disclosure comprises heavy chain CDR and light chain CDR sequence pairs (GR-HC CDRs/GR-LC CDRs) of: 3S-6, 3S-7, 3S-8/3S-9, 3S-10, 3S-11.

In certain embodiments, an antibody or antigen-binding fragment thereof of the disclosure comprises a 3S-HCVR/3S-LCVR sequence pair of: 3S-3/3S-5; 3S-13/3S-15; or 3S-VH 13/3S-VL 5.

In certain embodiments, an antibody or antigen-binding fragment thereof of the disclosure comprises a 3S-HCVR/3S-LCVR sequence pair of: 3S-VH 13/3S-VL 5.

In certain embodiments, an antibody or antigen-binding fragment thereof of the disclosure comprises a 3S heavy chain (HC)/3S light chain (LC) pair of: 3S-16/3S-18; 3S-17/3S-18; or 3S-19/3S-18.

In certain embodiments, an antibody or antigen-binding fragment thereof of the disclosure comprises the 3S-HCVR/3S-LCVR pair of anti-IL-4Rα monoclonal antibody SSGJ-611.

The antibodies recited below in Table 13 are described in more detail in U.S. Pat. No. 11,667,717, incorporated herein by reference in its entirety for all purposes.

TABLE 13

| | |
|---|---|
| 3S-6 | DDYIN |
| 3S-7 | WIFPGNGNSYYNEKFKD |
| 3S-8 | GLVRYRALFDY |
| 3S-9 | RASSSINYMH |
| 3S-10 | AASNLAS |
| 3S-11 | QQWSSYPIT |
| 3S-3 | QVQLQQSGPELVKPGASVKISCTASGSTLTDDYINW |
| | VKQRPGRGLEWVGWIFPGNGNSYYNEKFKDKATLIV |
| | DKSSSTAYMLLSSLTSEDSAVYFCARGLVRYRALFDY |
| | WGQGTTLTVSS |
| 3S-5 | QIVLSQSPAILSASPGEKVTMTCRASSSINYMHWYQQK |
| | PGSSPKPWIYAASNLASGVPARFSGSGSGTSFSLTISR |
| | VEAEDAATYYCQQWSSYPITFGSGTKLEIK |
| 3S-13 | QVQLVQSGAEVKKPGASVKVSCKASGSTLTDDYINW |
| | VRQAPGQRLEWVGWIFPGNGNSYYNEKFKDRATLTV |
| | DKSASTAYMELSSLRSEDTAVYFCARGLVRYRALFDY |
| | WGQGTLVTVSS |
| 3S-15 | DIQMTQSPSSLSASVGDRVTITCRASSSINYMHWYQQ |
| | KPGKAPKPWIYAASNLASGVPSRFSGSGSGTDFTLTIS |
| | SLQPEDFATYYCQQWSSYPITFGQGTKVEIK |
| 3S-16 | QVQLVQSGAEVKKPGASVKVSCKASGSTLTDDYINW |
| | VRQAPGQRLEWVGWIFPGNGNSYYNEKFKDRATLTV |
| | DKSASTAYMELSSLRSEDTAVYFCARGLVRYRALFDY |
| | WGQGTLVTVSSASTKGPSVFPLAPSSKSTSGGTAALG |
| | CLVKDYFPEPVTVSWNSGALTSGVHTFPAVLQSSGLY |
| | SLSSVVTVPSSSLGTQTYICNVNHKPSNTKVDKKVEPK |
| | SCDKTHTCPPCPAPELLGGPSVFLFPPKPKDTLMISRT |
| | PEVTCVVVDVSHEDPEVKFNWYVDGVEVHNAKTKPRE |
| | EQYNSTYRVVSVLTVLHQDWLNGKEYKCKVSNKALP |
| | APIEKTISKAKGQPREPQVYTLPPSREEMTKNQVSLTC |
| | LVKGFYPSDIAVEWESNGQPENNYKTTPPVLDSDGSFF |
| | LYSKLTVDKSRWQQGNVFSCSVMHEALHNHYTQKSL |
| | SLSPGK |
| 3S-17 | QVQLVQSGAEVKKPGASVKVSCKASGSTLTDDYINWV |
| | RQAPGQRLEWVGWIFPGNGNSYYNEKFKDRATLTVDK |
| | SASTAYMELSSLRSEDTAVYFCARGLVRYRALFDYWGQ |
| | GTLVTVSSASTKGPSVFPLAPCSRSTSESTAALGCLVK |
| | DYFPEPVTVSWNSGALTSGVHTFPAVLQSSGLYSLSSV |
| | VTVPSSSLGTKTYTCNVDHKPSNTKVDKRVESKYGPPC |
| | PPCPAPEFLGGPSVFLFPPKPKDTLMISRTPEVTCVVV |
| | DVSQEDPEVQFNWYVDGVEVHNAKTKPREEQFNSTYRV |
| | VSVLTVLHQDWLNGKEYKCKVSNKGLPSSIEKTISKAK |
| | GQPREPQVYTLPPSQEEMTKNQVSLTCLVKGFYPSDIA |
| | VEWESNGQPENNYKTTPPVLDSDGSFFLYSRLTVDKSR |
| | WQEGNVFSCSVMHEALHNHYTQKSLSLSLGK |
| 3S-19 | QVQLVQSGAEVKKPGASVKVSCKASGSTLTDDYINWVR |
| | QAPGQRLEWVGWIFPGNGNSYYNEKFKDRATLTVDKSA |
| | STAYMELSSLRSEDTAVYFCARGLVRYRALFDYWGQGT |
| | LVTVSSASTKGPSVFPLAPSSKSTSGGTAALGCLVKDY |
| | FPEPVTVSWNSGALTSGVHTFPAVLQSSGLYSLSSVVT |
| | VPSSSLGTQTYICNVNHKPSNTKVDKKVEPKSCDKTHT |
| | CPPCPAPELLGGPSVFLFPPKPKDTLMISRTPEVTCVV |
| | VDVEHEDPEVKFNWYVDGVEVHNAKTKPREEQYNSTYR |
| | VVSVLTVLHQDWLNGKEYKCKVSNKAFPAPIEKTISKA |
| | KGQPREPQVYTLPPSREEMTKNQVSLTCLVKGFYPSDI |
| | AVEWESNGQPENNYKTTPPVLDSDGSFFLYSKLTVDKS |
| | RWQQGNVFSCSVMHEALHNHYTQKSLSLSPGK |
| 3S-18 | DIQMTQSPSSLSASVGDRVTITCRASSSINYMHWYQQK |
| | PGKAPKPWIYAASNLASGVPSRFSGSGSGTDFTLTISS |
| | LQPEDFATYYCQQWSSYPITFGQGTKVEIKRTVAAPSV |
| | FIFPPSDEQLKSGTASVVCLLNNFYPREAKVQWKVDNA |
| | LQSGNSQESVTEQDSKDSTYSLSSTLTLSKADYEKHKV |
| | YACEVTHQGLSSPVTKSFNRGEC |
| 3S-VH 13 | QVQLVQSGAEVKKPGASVKVSCKASGSTLTDDYINW |
| | VRQAPGQRLEWVGWIFPGNGNSYYNEKFKDRATLTV |
| | DKSASTAYMELSSLRSEDTAVYFCARGLVRYRALFDY |
| | WGQGTLVTVSS |
| 3S-VL 5 | QIVLSQSPAILSASPGEKVTMTCRASSSINYMHW |
| | YQQKPGSSPKPWIYAASNLASGVPARFSGSGSGTSF |
| | SLTISRVEAEDAATYYCQQWSSYPITFGSGTKLEIK |

In certain embodiments, an antibody or antigen-binding fragment thereof of the disclosure comprises heavy chain CDR and light chain CDR sequence pairs (BA-HC CDRs/BA-LC CDRs) of: BA-14, BA-15, BA-16/BA-11, BA-12, BA-13; or BA-6, BA-7, BA-8/BA-3, BA-4, BA-5.

In certain embodiments, an antibody or antigen-binding fragment thereof of the disclosure comprises a BA-HCVR/BA-LCVR sequence pair of: BA-VH 2/BA-VL 1. In certain embodiments, an antibody or antigen-binding fragment thereof of the disclosure comprises a BA-HCVR/BA-LCVR sequence pair of: BA-VH 10/BA-VL 9 (e.g., of BA173).

In certain embodiments, an antibody or antigen-binding fragment thereof of the disclosure comprises the BA-HCVR/BA-LCVR pair of anti-IL-4Rα monoclonal antibody BA2101.

The antibodies recited below in Table 14 are described in more detail in US 2022/0162328 and EP3978528A1, incorporated herein by reference in their entireties for all purposes.

TABLE 14

| | |
|---|---|
| BA-3 | QNIGSR |
| BA-4 | KAS |
| BA-5 | QQYNSYSWT |
| BA-6 | GFTFSSYA |
| BA-7 | ISYDGSNK |
| BA-8 | ARGLTTVRGVLY |
| BA-11 | QSISTR |
| BA-12 | WAS |
| BA-13 | QQYTSYSWT |
| BA-14 | GFTFSSYA |
| BA-15 | ISYDGSNK |
| BA-16 | ARGLTTVRGVLY |
| BA-VH 2 | QVQLVESGGGVVQPGRSLRLSCAASGFTFSSYAM |
| | HWVRQAPGKGLEWVAVISYDGSNKYYADSVKGR |
| | FTISRDNSKNTLYLQMNSLRAEDTAVYYCARGLTT |
| | VRGVLYWGQGTLVTVSS |
| BA-VL 1 | EIVMTQSPSSLSASLGDRVTITCRASQNIGSRLAWY |
| | QQKPGKAPKLLIYKASSLESGVPSRFSGSGSGTEFT |
| | LTISSLQPDDFATYYCQQYNSYSWTFGQGTKLEIK |
| BA-VL 9 | DIVMTQSPSTLSASVGDRVTITCRASQSISTRLAWYQQ |
| | KPGKAPKLLVYWASSLESGVPSRFSGSGSGTEFTLA |
| | ISSLQPDDFGTYYCQQYTSYSWTFGQGTKLEIK |
| BA-VH 10 | QVQLVESGGGVVQPGRSLRLSCAASGFTFSSYAMH |
| | WVRQAPGKGLEWVAVISYDGSNKYYADSVKGRFTI |
| | SRDNSKNTLYLQMNSLRAEDTAVYYCARGLTTVRGV |
| | LYWGQGTLVTVSS |

In certain embodiments, an antibody or antigen-binding fragment thereof of the disclosure comprises a TQH-HCVR/TQH-LCVR sequence pair of: TQH-VH 43/TQH-VL 46 (e.g., of BSG11F2B7G5E8-V11). In certain embodiments, an antibody or antigen-binding fragment thereof of the disclosure comprises a TQH-HCVR/TQH-LCVR sequence pair of: TQH-VH 44/TQH-VL 46 (e.g., of BSG11F2B7G5E8-V13). In certain embodiments, an antibody or antigen-binding fragment thereof of the disclosure comprises a TQH-HCVR/TQH-LCVR sequence pair of: TQH-VH 47/TQH-VL 48 (e.g., of B8D10G7G6E4). In certain embodiments, an antibody or antigen-binding fragment thereof of the disclosure comprises a TQH-HCVR/TQH-LCVR sequence pair of: TQH-VH 49/TQH-VL 50 (e.g., of B9A7C9A4H5). In certain embodiments, an antibody or antigen-binding fragment thereof of the disclosure comprises a TQH-HCVR/TQH-LCVR sequence pair of: TQH-VH 51/TQH-VL 52 (e.g., of B9D1D11F8D8). In certain embodiments, an antibody or antigen-binding fragment thereof of the disclosure comprises a TQH-HCVR/TQH-LCVR sequence pair of: TQH-VH 53/TQH-VL 54 (e.g., of B1D2F7D3B5).

In certain embodiments, an antibody or antigen-binding fragment thereof of the disclosure comprises the heavy chain CDRs of TQH-VH 33 and the light chain CDRs of TQH-VL 46.

In certain embodiments, an antibody or antigen-binding fragment thereof of the disclosure comprises a BA-HCVR/BA-LCVR sequence pair of: TQH-VH 33/TQH-VL 46.

In certain embodiments, an antibody or antigen-binding fragment thereof of the disclosure comprises the TQH-HCVR/TQH-LCVR pair of anti-IL-4Rα monoclonal antibody TQH2722.

The antibodies recited below in Table 15 are described in more detail in US 2023/0105029, incorporated herein by reference in its entirety for all purposes.

TABLE 15

| | |
|---|---|
| TQH-VH 33 | EVQLVESGGGLVQPGGSLRLSCAASGFTFSTYGMSW |
| | VRQAPGKGLVXVXTINSNGGSTSYPDSVKGRFTISRD |
| | NAKNTLYLQMNSLRAEDTAVYYCARFFRFRNAMDY |
| | WGQGTLVTVSS |
| TQH-VH 43 | EVQLVQSGAEVKKPGATVKISCKVSGFNIKDTYMHW |
| | VQQAPGKGLEWMGLIDPTNGYTIYAEKFQGRVTITAD |
| | TSTDTAYMELSSLRSEDTAVYYCVSRRPWFAYWGQGT |
| | LVTVSS |
| TQH-VH 44 | QVQLQQSGAELVKPGASVKLSCTASGFNIKDTYMHW |
| | VKQRPEQGLEWVGRIDPTNGYTIYASKFQGKATITAD |
| | TSSNTAYMQLSSLTSEDTAVYHCVSRRPWFAYWGQGT |
| | TLTVSS |
| TQH-VH 47 | EVQLVESGGGLVKPGGSLKLSCAASGFTFSSYAMSW |
| | VRQTPEKRLEWVAGIRSGGSYTYYPDTVKGRFTISRD |
| | NARNTLYLQMNSLRSEDTAIYYCARGDKLRPYHFDYW |
| | GQGTTLTVSS |
| TQH-VH 49 | EVQLVESGGGLVKPGGSLKLSCAASGFTFSNYAMSW |
| | VRQTPEKRLEWVAGIRSGGSYTYYPDTVKGRFTISRD |
| | NARNTLYLQMSSLRSEDTAIYYCARGDKLRPYHFDYW |
| | GQGTTLTVSS |
| TQH-VH 51 | EVKLVESGGGLVKPGGSLKLSCAASGFTFSSYAMSW |
| | VRQTPEKRLEWVASISSGDSTYYLDSVKGRFTISRD |
| | NARNILYLQVSSLRSEDTAMYYCERSGGSAPYWGQG |
| | TLVTVSA |
| TQH-VH 53 | EVKLVESGGGLVKPGGSLKLSCAASGFTFSSYAMSW |
| | VRQTPEKRLEWVASISSGDSTYYLDSVKGRFTISRD |
| | NAMNILYLQMSSLRSEDTAVYYCERSGGSAPYWGQG |
| | TLVSVSA |
| TQH-VL 46 | DIVMTQTPLSLSVTPGQPASISCRSSQSIVHSNGNTYL |
| | EWYLQKPGQSPQLLIYKVTNRFSGVPDRFSGSGSGTDF |
| | TLKISRVEAEDVGVYYCFQGSHVPYTFGQGTKLEIK |
| TQH-VL 48 | DIVMTQSHKFMSTSVGDKVSITCKASQDVTTAVAW |
| | YQQKPGQSPKLLIYSASYRYTGVPDRFAGSGSGTDF |
| | TVTISTVQAEDLAVYYCQQHYSDPYTFGGGTKLEIK |
| TQH-VL 50 | DIVMTQSHKFMSTSVGDRVSITCKASQDVSTAVVW |
| | YQQKPGQSPKLLIYSASYRYTGVPDRFTGSGSGTDF |
| | TFTIITVQAEDLAVYYCQQHYSAPYTFGGGTQLEIK |
| TQH-VL 52 | QIVLTQSPAIMSASPGDMVTISCSASSSVNYMYW |
| | YQQKPGSSPKPWIYRTSNLASGVPARFSGSGSGTSY |
| | SLTISSMEAEDAATYYCQQYHSFPLTFGAGTKLELK |
| TQH-VL 54 | QIVLTQSPAIMSASPGEMVTISCSASSSVSYMYW |
| | YQQKPGSSPKPWIYRTSNLASGVPARFSGSGSGTSY |
| | SLTISSMEAEDAATYYCQQYHSFPLTFGAGTKLELK |

In certain embodiments, an antibody or antigen-binding fragment thereof of the disclosure comprises a mAb422-HCVR/mAb422-LCVR sequence pair of Ab422-HCVR 121/mAb422-LCVR 158. In certain embodiments, an antibody or antigen-binding fragment thereof of the disclosure comprises a mAb471-HCVR/mAb471-LCVR sequence pair of mAb472-HCVR 134/mAb47-LCVR 178.

In certain embodiments, an antibody or antigen-binding fragment thereof of the disclosure comprises three heavy chain CDRs of mAb422 2, mAb422 10, mAb422 20, and three light chain CDRs of mAb422 27, mAb422 43, mAb422 61.

In certain embodiments, an antibody or antigen-binding fragment thereof of the disclosure comprises three heavy chain CDRs of mAb471 2, mAb471 6, mAb471 20, and three light chain CDRs of mAb471 30, mAb471 47, mAb471 61.

The antibodies recited below in Table 16 are described in more detail in WO 2024/173847 A2, incorporated herein by reference in its entirety for all purposes.

TABLE 16

| | |
|---|---|
| mAb422- | EVQLVESGGGLEQPGGSLRLSCAGSGFTFRDFAMTWVR |
| HCVR 121 | QAPGKGLEWVSSVSGSGGNTYYADSVKGRFTISRDNSK |
| | NTLYLQMNSLRAEDTAVYYCAKDRLSVTVRPRYYGLDV |
| | WGQGTTVTVSS |
| mAb422- | DIVMTQSPLSLPVTPGEPASISCRSSQSVLYSIGYNYV |
| LCVR 158 | DWYLQKSGQSPQLLIYLGNKRASGVPDRFSGSGSGTDF |
| | TLKISRVEAEDVGFYYCMQSLTSPYTFGQGTKLEIK |
| mAb471- | EVQLVESGGGLEQPGGSLRLSCAGSGFTFRDFAMTWVR |
| HCVR 134 | QAPGKGLEWVSSISGSGGNIYYADSVKGRFTISRDNSK |
| | NTLYLQMNSLRAEDTAVYYCAKDRLSVTVRPRYYGLDV |
| | WGQGTTVTVSS |
| mAb471- | DIVMTQSPLSLPVTPGEPASISCRSSQSLLYSIGKNYV |
| LCVR 178 | DWYLQKSGQSPQLLIYEGNKRASGVPDRFSGSGSGTDF |
| | TLKISRVEAEDVGFYYCMQSLTSPYTFGQGTKLEIK |
| mAb422 2 | DFAMT |
| mAb422 10 | SVSGSGGNTYYADSVKG |
| mAb422 20 | DRLSVTVRPRYYGLDV |
| mAb422 27 | RSSQSVLYSIGYNYVD |
| mAb422 43 | LGNKRAS |
| mAb422 61 | MQSLTSPYT |
| mAb471 2 | DFAMT |
| mAb471 6 | SISGSGGNIYYADSVKG |
| mAb471 20 | DRLSVTVRPRYYGLDV |
| mAb471 30 | RSSQSLLYSIGKNYVD |
| mAb471 47 | EGNKRAS |
| mAb471 61 | MQSLTSPYT |

In certain embodiments, an antibody or antigen-binding fragment thereof of the disclosure comprises any BK-HCVR/BK-LCVR sequence pair listed in Table 17. In certain embodiments, an antibody or antigen-binding fragment thereof of the disclosure comprises a BK-HCVR/BK-LCVR sequence pair selected from the group consisting of BK-HCVR 1/BK-LCVR 3, BK-HCVR 1/BK-LCVR 5, BK-HCVR 1/BK-LCVR 7, BK-HCVR 1/BK-LCVR 9, BK-HCVR 11/BK-LCVR 2, BK-HCVR 13/BK-LCVR 2, BK-HCVR 15/BK-LCVR 2, BK-HCVR 17/BK-LCVR 2, BK-HCVR 19/BK-LCVR 2, BK-HCVR 21/BK-LCVR 2, BK-HCVR 23/BK-LCVR 2, and BK-HCVR 25/BK-LCVR 2.

In certain embodiments, an antibody or antigen-binding fragment thereof of the disclosure comprises any CDRs from a BK-HCVR/BK-LCVR sequence pair listed in Table 17. In certain embodiments, an antibody or antigen-binding fragment thereof of the disclosure comprises the CDRs from a BK-HCVR/BK-LCVR sequence pair selected from the group consisting of BK-HCVR 1/BK-LCVR 3, BK-HCVR 1/BK-LCVR 5, BK-HCVR 1/BK-LCVR 7, BK-HCVR 1/BK-LCVR 9, BK-HCVR 11/BK-LCVR 2, BK-HCVR 13/BK-LCVR 2, BK-HCVR 15/BK-LCVR 2, BK-HCVR 17/BK-LCVR 2, BK-HCVR 19/BK-LCVR 2, BK-HCVR 21/BK-LCVR 2, BK-HCVR 23/BK-LCVR 2, and BK-HCVR 25/BK-LCVR 2.

The antibodies recited below in Table 17 are described in more detail in WO 2024/173847 A2, incorporated herein by reference in its entirety for all purposes.

TABLE 17

| | |
|---|---|
| BK-HCVR 1 | GVGLVGSGGGLGGPGGSLALSCAGSGPTPAATAMTT |
| | VAGAPGLGLGTVSSISGSGGATTT |
| | AASVLGAPTISAAASLATLTLGMASLAAGATAVTTC |
| | ALAALSITIAPATTGLAVTGGGTT |
| | VTVS |
| BK-HCVR 11 | GVGLVGSGGGLGGPGGSLALSCAGSGPTPAATAMTT |
| | VAGAPGLGLGTVSSISGSGGATTT |
| | AASVLGAPTISAAASLATLTLGMASLAAGATAVTTC |
| | ALAALSITIAPATTGLAVTGGGTT |
| | VTVS |
| BK-HCVR 13 | GVGLVGSGGGLGGPGGSLALSCAGSGPTPAATAMTT |
| | VAGAPGLGLGTVSSISGSGGATTT |
| | AASVLGAPTISAAASLATLTLGMASLAAGATAVTTC |
| | ALAALSITIAPATTGLAVTGGGTT |
| | VTVS |
| BK-HCVR 15 | GVGLVGSGGGLGGPGGSLALSCAGSGPTPAATAMTT |
| | VAGAPGLGLGTVSSISGSGGATTT |
| | AASVLGAPTISAAASLATLTLGMASLAAGATAVTTC |
| | ALAALSITIAPATTGLAVTGGGTT |
| | VTVS |
| BK-HCVR 17 | GVGLVGSGGGLGGPGGSLALSCAGSGPTPAATAMTT |
| | VAGAPGLGLGTVSSISGSGGATTT |
| | AASVLGAPTISAAASLATLTLGMASLAAGATAVTTC |
| | ALAALSITIAPATTGLAVTGGGTT |
| | VTVS |
| BK-HCVR 19 | GVGLVGSGGGLGGPGGSLALSCAGSGPTPAATAMTT |
| | VAGAPGLGLGTVSSISGSGGATTT |
| | AASVLGAPTISAAASLATLTLGMASLAAGATAVTTC |
| | ALAALSITIAPATTGLAVTGGGTT |
| | VTVS |
| BK-HCVR 21 | GVGLVGSGGGLGGPGGSLALSCAGSGPTPAATAMTT |
| | VAGAPGLGLGTVSSISGSGGATTT |
| | AASVLGAPTISAAASLATLTLGMASLAAGATAVTTC |
| | ALAALSITIAPATTGLAVTGGGTT |
| | VTVS |
| BK-HCVR 23 | GVGLVGSGGGLGGPGGSLALSCAGSGPTPAATAMTT |
| | VAGAPGLGLGTVSSISGSGGATTT |
| | AASVLGAPTISAAASLATLTLGMASLAAGATAVTTC |
| | ALAALSITIAPATTGLAVTGGGTT |
| | VTVS |
| BK-HCVR 25 | GVGLVGSGGGLGGPGGSLALSCAGSGPTPAATAMTT |
| | VAGAPGLGLGTVSSISGSGGATTT |
| | AASVLGAPTISAAASLATLTLGMASLAAGATAVTTC |
| | ALAALSITIAPATTGLAVTGGGTT |
| | VTVS |
| BK-LCVR 2 | AIVMTGSPLSLPVTPGGPASISCASSGSLLTSIGTA |
| | TLATTLGLSGGSPGLLITLGSAAA |
| | SGVPAAPSGSGSGTAPTLLISAVGAGAVGPTTCMGA |
| | LGTPTTPGGGTLLGIL |
| BK-LCVR 3 | AIVMTGSPLSLPVTPGGPASISCASSGSLLTSIGTA |
| | TLATTLGLSGGSPGLLITLGSAAA |
| | SGVPAAPSGSGSGTAPTLLISAVGAGAVGPTTCMGA |
| | LGTPTTPGGGTLLGIL |
| BK-LCVR 5 | AIVMTGSPLSLPVTPGGPASISCASSGSLLTSIGTA |
| | TLATTLGLSGGSPGLLITLGSAAA |
| | SGVPAAPSGSGSGTAPTLLISAVGAGAVGPTTCMGA |
| | LGTPTTPGGGTLLGIL |
| BK-LCVR 7 | AIVMTGSPLSLPVTPGGPASISCASSGSLLTSIGTA |
| | TLATTLGLSGGSPGLLITLGSAAA |
| | SGVPAAPSGSGSGTAPTLLISAVGAGAVGPTTCMGA |
| | LGTPTTPGGGTLLGIL |
| BK-LCVR 9 | AIVMTGSPLSLPVTPGGPASISCASSGSLLTSIGTA |
| | TLATTLGLSGGSPGLLITLGSAAA |
| | SGVPAAPSGSGSGTAPTLLISAVGAGAVGPTTCMGA |
| | LGTPTTPGGGTLLGIL |

### Pharmaceutical Compositions

Methods that comprise administering an IL-4R antagonist to a patient, wherein the IL-4R antagonist is contained within a pharmaceutical composition are provided. The pharmaceutical compositions described herein are formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol. 52:238-311.

The dose of antibody administered to a patient may vary depending upon the age and the size of the patient, symptoms, conditions, route of administration, and the like. The dose is typically calculated according to body weight or body surface area. Depending on the severity of the condition, the frequency and the duration of the treatment can be adjusted. Effective dosages and schedules for administering pharmaceutical compositions comprising anti-IL-4R antibodies may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical compositions described herein, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of administration include, but are not limited to, intradermal, intramuscular, intraperitoneal, intravenous, subcutaneous, intranasal, intra-tracheal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents.

A pharmaceutical composition described herein can be delivered subcutaneously or intravenously with a standard needle and syringe. In addition, with respect to subcutaneous delivery, a pen delivery device (e.g., an autoinjector pen) readily has applications in delivering a pharmaceutical composition described herein. Such a pen delivery device can be reusable or disposable. A reusable pen delivery device generally utilizes a replaceable cartridge that contains a pharmaceutical composition. Once all the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition described herein include, but are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.), and the HUMIRA™ Pen (Abbott Labs, Abbott Park IL), to name only a few. Examples of large-volume delivery devices (e.g., large-volume injectors) include, but are not limited to, bolus injectors such as, e.g., BD Libertas West SMARTDOSE™, Enable Injections, STEADYMED PATCHPUMP™ SENSILE SENSETRIAL™, YPSOMED YPSODOSE™, BESPAK LAPAS™, and the like.

An example drug delivery device may involve a needle-based injection system. Needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

An example sleeve-triggered auto-injector with manual needle insertion is described in International Publication WO2015/004052. Example audible end-of-dose feedback mechanisms are described in International Publications WO2016/193346 and WO2016/193348. An example needle-safety mechanism after using an auto-injector is described in International Publication WO2016/193352. An example needle sheath remover mechanism for a syringe auto-injector is described in International Publication WO2016/193353. An example support mechanism for supporting an axial position of a syringe is described in International Publication WO2016/193355.

For direct administration to the sinuses, the pharmaceutical compositions described herein may be administered using, e.g., a microcatheter (e.g., an endoscope and microcatheter), an aerosolizer, a powder dispenser, a nebulizer or an inhaler. The methods include administration of an IL-4R antagonist to a subject in need thereof, in an aerosolized formulation. For example, aerosolized antibodies to IL-4R may be administered to treat CRSsNP in a patient. Aerosolized antibodies can be prepared as described in, for example, U.S. Pat. No. 8,178,098, incorporated herein by reference in its entirety.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant (e.g., polysorbate 80, HCO-50 (polyoxyethylene (50 mol) adduct of hydrogenated castor oil)), etc. As the oily medium, there are employed, e.g., sesame oil, soybean oil, etc., which may be used in combination with a solubilizing agent such as benzyl benzoate, benzyl alcohol, etc. The injection thus prepared is typically filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

Exemplary pharmaceutical compositions comprising an anti-IL-4R antibody that can be used as described herein are disclosed, e.g., in U.S. Pat. No. 8,945,559.

### Dosage

The amount of IL-4R antagonist (e.g., an anti-IL-4R antibody or antigen-binding fragment thereof) administered to a subject according to the methods featured in the disclosure or for use according to the disclosure is, generally, a therapeutically effective amount. As used herein, the phrase “therapeutically effective amount” means an amount of IL-4R antagonist that results in one or more of: (a) a reduction in the incidence of CRSsNP exacerbations; (b) an improvement in one or more CRSsNP-associated parameters (as defined elsewhere herein); and/or (c) a detectable improvement in one or more symptoms or indicia of an upper airway inflammatory condition. A “therapeutically effective amount” also includes an amount of IL-4R antagonist that inhibits, prevents, lessens, or delays the progression of CRSsNP in a subject.

In the case of an anti-IL-4R antibody, a therapeutically effective amount can be from about 0.05 mg to about 700 mg, e.g., about 0.05 mg, about 0.1 mg, about 1.0 mg, about 1.5 mg, about 2.0 mg, about 3.0 mg, about 5.0 mg, about 7.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, or about 700 mg of the anti-IL-4R antibody. In certain embodiments, about 300 mg of an anti-IL-4R antibody is administered. In certain embodiments, about 200 mg of an anti-IL-4R antibody is administered.

The amount of IL-4R antagonist contained within the individual doses may be expressed in terms of milligrams of antibody per kilogram of subject body weight (i.e., mg/kg). For example, the IL-4R antagonist may be administered to a patient at a dose of about 0.0001 to about 10 mg/kg of subject body weight. For example, the IL-4R antagonist can be administered at a dose of 1 mg/kg, 2 mg/kg, 3 mg/kg, 4 mg/kg, 5 mg/kg or 6 mg/kg.

In certain embodiments, the initial dose is about the same as the loading dose. In certain embodiments, the initial dose is about 1.1×, about 1.2×, about 1.3×, about 1.4×, about 1.5×, about 1.6×, about 1.7×, about 1.8×, about 1.9×, about 2.0×, about 2.5×, about 3.0×, or more of the loading dose.

In certain embodiments, two or more (e.g., 2, 3, 4, or 5 or more) doses are administered at the beginning of the treatment regimen as “initial doses” or “loading doses” followed by subsequent doses that are administered on a less frequent basis (e.g., “secondary doses” or “maintenance doses”). In one embodiment, the maintenance dose may be lower than the loading or initial dose. For example, one or more initial doses of 600 mg of IL-4R antagonist may be administered followed by maintenance doses of about 75 mg to about 300 mg. In certain embodiments, the methods comprise an initial dose or loading dose of about 400 mg or about 600 mg of an IL-4R antagonist. In certain embodiments, the methods comprise one or more secondary doses or maintenance doses of about 200 mg or about 300 mg of the IL-4R antagonist.

In one embodiment, the maintenance dose is the same dose as the loading or initial dose. For example, both the loading dose and the maintenance doses of the IL-4R antagonist may be administered in doses of about 75 mg to about 300 mg. In certain embodiments, the methods comprise an initial dose and maintenance doses of about 300 mg of an IL-4R antagonist.

In certain exemplary embodiments, a subject is a pediatric subject having a body weight of more than 30 kg, and the IL-4R antagonist is administered at a dose of about 150 mg, about 200 mg, about 250 mg, about 300 mg, about 350 mg, about 400 mg, about 450 mg, about 500 mg, about 550 mg, or about 600 mg. In some embodiments, a subject is a pediatric subject having a body weight of more than 30 kg, and the IL-4R antagonist is administered at an initial dose or loading dose of about 400 mg and one or more secondary doses or maintenance doses of about 200 mg, and the secondary doses are administered every other week (q2w). In some embodiments, a subject is a pediatric subject having a body weight of more than 30 kg, and the IL-4R antagonist is administered at an initial dose and maintenance doses of about 200 mg, and the maintenance doses are administered every other week (q2w).

In certain exemplary embodiments, a subject is a pediatric subject having a body weight of 30 kg or less and a body weight of at least 15 kg, and the IL-4R antagonist is administered at a dose of about 50 mg, about 100 mg, about 150 mg, about 200 mg, about 250 mg, about 300 mg, about 350 mg, about 400 mg, about 450 mg, about 500 mg, about 550 mg, or about 600 mg. In some embodiments, a subject is a pediatric subject having a body weight of 30 kg or less and a body weight of at least 15 kg, and the IL-4R antagonist is administered at an initial dose of about 600 mg and one or more secondary doses or maintenance doses of about 300 mg, and the secondary doses are administered every four weeks (q4w). In some embodiments, a subject is a pediatric subject having a body weight of 30 kg or less and a body weight of at least 15 kg, and the IL-4R antagonist is administered at an initial dose and maintenance doses of about 300 mg, and the maintenance doses are administered every four weeks (q4w).

In certain exemplary embodiments, a subject is an adolescent subject having a body weight of less than 60 kg, and the IL-4R antagonist is administered at a dose of about 50 mg, about 100 mg, about 150 mg, about 200 mg, about 250 mg, about 300 mg, about 350 mg, about 400 mg, about 450 mg, about 500 mg, about 550 mg, or about 600 mg. In some embodiments, a subject is an adolescent subject having a body weight of less than 60 kg, and the IL-4R antagonist is administered at an initial dose of about 400 mg and one or more secondary doses or maintenance doses of about 200 mg, and the secondary doses are administered every other week (q2w). In other embodiments, a subject is an adolescent subject having a body weight of less than 60 kg, and the IL-4R antagonist is administered at an initial dose and maintenance doses of about 200 mg, and the maintenance doses are administered every other week (q2w). In certain embodiments, a subject is an adolescent subject having a body weight that is greater than or equal to 30 kg and less than 60 kg, and the IL-4R antagonist is administered at a dose of about 50 mg, about 100 mg, about 150 mg, about 200 mg, about 250 mg, about 300 mg, about 350 mg, about 400 mg, about 450 mg, about 500 mg, about 550 mg, or about 600 mg. In some embodiments, a subject is an adolescent subject having a body weight that is greater than or equal to 30 kg and less than 60 kg, and the IL-4R antagonist is administered at an initial dose of about 400 mg and one or more secondary doses or maintenance doses of about 200 mg, and the secondary doses are administered every other week (q2w). In other embodiments, a subject is an adolescent subject having a body weight that is greater than or equal to 30 kg and less than 60 kg, and the IL-4R antagonist is administered at an initial dose and maintenance doses of about 200 mg, and the maintenance doses are administered every other week (q2w).

In certain exemplary embodiments, a subject is an adolescent subject having a body weight of at least 60 kg, and the IL-4R antagonist is administered at a dose of about 50 mg, about 100 mg, about 150 mg, about 200 mg, about 250 mg, about 300 mg, about 350 mg, about 400 mg, about 450 mg, about 500 mg, about 550 mg, or about 600 mg. In exemplary embodiments, a subject is an adolescent subject having a body weight of at least 60 kg, and the IL-4R antagonist is administered at an initial dose of about 600 mg and one or more secondary doses or maintenance doses of about 300 mg, and the secondary doses are administered every other week (q2w). In other embodiments, a subject is an adolescent subject having a body weight of at least 60 kg, and the IL-4R antagonist is administered at an initial dose and maintenance doses of about 300 mg, and the maintenance doses are administered every other week (q2w). In other embodiments, a subject is an adolescent subject having a body weight of greater than or equal to 60 kg, and the IL-4R antagonist is administered at an initial dose and maintenance doses of about 300 mg, and the maintenance doses are administered every other week (q2w). In other embodiments, a subject is an adolescent subject having a body weight of greater than or equal to 30 kg and less than 60 kg, and the IL-4R antagonist is administered at an initial dose and maintenance doses of about 200 mg, and the maintenance doses are administered every other week (q2w).

In certain exemplary embodiments, a subject is an adult, and the IL-4R antagonist is administered at a dose of about 50 mg, about 100 mg, about 150 mg, about 200 mg, about 250 mg, about 300 mg, about 350 mg, about 400 mg, about 450 mg, about 500 mg, about 550 mg, or about 600 mg. In exemplary embodiments, a subject is an adult, and the IL-4R antagonist is administered at an initial dose of about 600 mg and one or more secondary doses or maintenance doses of about 300 mg, and the secondary doses are administered every other week (q2w). In other exemplary embodiments, a subject is an adult, and the IL-4R antagonist is administered at an initial dose of about 400 mg and one or more secondary doses or maintenance doses of about 200 mg, and the secondary doses are administered every other week (q2w). In certain embodiments, a subject is an adult, the initial dose comprises about 300 mg of the IL-4R antagonist, and the one or more subsequent doses comprise about 300 mg of the IL-4R antagonist administered every other week (q2w). In certain exemplary embodiments, the subject has an age between 40 years and 85 years.

In certain exemplary embodiments, an IL-4R antagonist is administered at a concentration of 150 mg/mL using a prefilled device. In some embodiments, a 150 mg/mL IL-4R antagonist solution in a pre-filled device is used to deliver about 300 mg IL-4R antagonist in a 2 mL injection. In certain exemplary embodiments, an IL-4R antagonist is administered at a concentration of 175 mg/mL using a prefilled device. In some embodiments, a 175 mg/mL IL-4R antagonist solution in a pre-filled device is used to deliver about 200 mg IL-4R antagonist in a 1.14 mL injection.

### Combination Therapies

Certain embodiments of the methods described herein comprise administering to the subject one or more additional therapeutic agents in combination with the IL-4R antagonist. As used herein, the expression “in combination with” means that the additional therapeutic agents are administered before, after, or concurrent with the pharmaceutical composition comprising the IL-4R antagonist. In some embodiments, the term “in combination with” includes sequential or concomitant administration of an IL-4R antagonist and a second therapeutic agent. Methods to treat CRSsNP or an associated condition or complication or to reduce at least one CRSsNP exacerbation comprising administration of an IL-4R antagonist in combination with a second therapeutic agent for additive or synergistic activity, are provided.

For example, when administered “before” the pharmaceutical composition comprising the IL-4R antagonist, the additional therapeutic agent may be administered about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes, or about 10 minutes prior to the administration of the pharmaceutical composition comprising the IL-4R antagonist. When administered “after” the pharmaceutical composition comprising the IL-4R antagonist, the additional therapeutic agent may be administered about 10 minutes, about 15 minutes, about 30 minutes, about 1 hour, about 2 hours, about 4 hours, about 6 hours, about 8 hours, about 10 hours, about 12 hours, about 24 hours, about 36 hours, about 48 hours, about 60 hours, or about 72 hours after the administration of the pharmaceutical composition comprising the IL-4R antagonist. Administration “concurrent” with the pharmaceutical composition comprising the IL-4R antagonist means that the additional therapeutic agent is administered to the subject in a separate dosage form within less than 5 minutes (before, after, or at the same time) of administration of the pharmaceutical composition comprising the IL-4R antagonist, or administered to the subject as a single combined dosage formulation comprising both the additional therapeutic agent and the IL-4R antagonist.

The additional therapeutic agent may be, e.g., another IL-4R antagonist, an IL-33 antagonist, an IL-1 antagonist (including, e.g., an IL-1 antagonist as set forth in U.S. Pat. No. 6,927,044), an IL-6 antagonist, an IL-6R antagonist (including, e.g., an anti-IL-6R antibody as set forth in U.S. Pat. No. 7,582,298), a TNF antagonist, an IL-8 antagonist, an IL-9 antagonist, an IL-17 antagonist, an IL-5 antagonist, an IgE antagonist, a CD48 antagonist, a leukotriene inhibitor, an anti-fungal agent, an NSAID, a long-acting muscarinic antagonist (LAMA), a long-acting beta₂ agonist (LABA), an inhaled corticosteroid (ICS), a systemic corticosteroid (e.g., oral or intravenous), methylxanthine, nedocromil sodium, cromolyn sodium, or combinations thereof. In exemplary embodiments, an additional therapeutic agent administered in combination with the IL-4R antagonist is a background therapy.

In certain embodiments, the pharmaceutical composition comprising an IL-4R antagonist is administered with a combination comprising a LABA, a LAMA, and an ICS. In other embodiments, an ICS is contraindicated and the pharmaceutical composition comprising an IL-4R antagonist is administered with a combination comprising a LABA and a LAMA.

In certain embodiments, the pharmaceutical composition comprising an IL-4R antagonist is administered with a background therapy comprising a LABA, a LAMA, and an ICS. In other embodiments, an ICS is contraindicated and the pharmaceutical composition comprising an IL-4R antagonist is administered with a background therapy comprising a LABA and a LAMA.

In certain embodiments, the pharmaceutical composition comprising an IL-4R antagonist is administered with a high dose ICS. In some embodiments, the pharmaceutical composition comprising an IL-4R antagonist is administered with a high dose ICS, a LAMA, and a LABA. In some embodiments, the high dose ICS is beclometasone dipropionate (CFC) and the dose is greater than 1000 mcg. In some embodiments, the high dose ICS is beclometasone dipropionate (HFA) and the dose is greater than 400 μg. In some embodiments, the high dose ICS is budesonide (DPI) and the dose is greater than 800 mcg. In some embodiments, the high dose ICS is ciclesonide (HFA) and the dose is greater than 320 mcg. In some embodiments, the high dose ICS is fluticasone propionate (DPI or HFA) and the dose is greater than 500 μg. In some embodiments, the high dose ICS is mometasone furoate and the dose is greater than 440 μg. In some embodiments, the high dose ICS is triamcinolone acetonide and the dose is greater than 2000 μg.

In certain embodiments, the pharmaceutical composition comprising an IL-4R antagonist is administered with a non-high dose ICS. In some embodiments, the pharmaceutical composition comprising an IL-4R antagonist is administered with a non-high dose ICS, a LAMA, and a LABA. In some embodiments, the non-high dose ICS is beclometasone dipropionate (CFC) and the dose is equal or less than 1000 mcg. In some embodiments, the non-high dose ICS is beclometasone dipropionate (HFA) and the dose is equal or below 400 μg. In some embodiments, the non-high dose ICS is budesonide (DPI) and the dose is equal or below 800 mcg. In some embodiments, the non-high dose ICS is ciclesonide (HFA) and the dose is equal or below 320 mcg. In some embodiments, the non-high dose ICS is fluticasone propionate (DPI or HFA) and the dose is equal or below 500 μg. In some embodiments, the non-high dose ICS is mometasone furoate and the dose is equal or below 440 μg. In some embodiments, the non-high dose ICS is triamcinolone acetonide and the dose is equal or below 2000 μg.

Suitable LABAs include, but are not limited to, salmeterol (e.g., SEREVENT®), formoterol (e.g., FORADIL®, PERFOROMIST®), indacaterol (e.g., ARCAPTA®), arformoterol (e.g., BROVANA®), olodaterol (e.g., STIVERDI®), and the like.

Suitable ICSs include, but are not limited to, fluticasone (e.g., fluticasone propionate, e.g., FLOVENT®), budesonide, mometasone (e.g., mometasone furoate, e.g., ASMANEX®), flunisolide (e.g., AEROBID®), dexamethasone acetate/phenobarbital/theophylline (e.g., AZMACORT®), beclomethasone dipropionate HFA (QVAR®), beclomethasone dipropionate (CFC), ciclesonide (HFA), triamcinolone acetonide and the like.

Suitable LAMAs include, but are not limited to, tiotropium bromide (e.g., SPIRIVA®), aclidinium bromide (e.g., EKLIRA®, TUDORZA®), glycopyrronium bromide (e.g., SEEBRI®), umeclidinium (e.g., INCRUSE®) and the like.

Suitable LAMA and LABA combinations include, but are not limited to, umeclidinium and vilanterol (e.g., Anoro), olodaterol and tiotropium (e.g., Stiolto), indacaterol and glycopyrrolate (e.g., Utibron), and glycopyrrolate and formoterol (e.g., Bevespi).

Suitable oral corticosteroids include, but are not limited to, prednisone, prednisolone, methylprednisolone, hydrocortisone, dexamethasone, cortisone acetate and the like.

### Administration Regimens

According to certain embodiments, multiple doses of an IL-4R antagonist may be administered to a subject over a defined time course. Such methods comprise sequentially administering to a subject multiple doses of an IL-4R antagonist. As used herein, “sequentially administering” means that each dose of IL-4R antagonist is administered to the subject at a different point in time, e.g., on different days separated by a predetermined interval (e.g., hours, days, weeks, or months). Methods that comprise sequentially administering to the patient a single initial dose of an IL-4R antagonist, followed by one or more secondary doses of the IL-4R antagonist, and optionally followed by one or more tertiary doses of the IL-4R antagonist, are provided.

Methods comprising administering to a subject a pharmaceutical composition comprising an IL-4R antagonist at a dosing frequency of about four times a week, twice a week, once a week (qw or q1w), once every two weeks (every two weeks is used interchangeably with every other week, bi-weekly or q2w), once every three weeks (tri-weekly or q3w), once every four weeks (monthly or q4w), once every five weeks (q5w), once every six weeks (q6w), once every seven weeks (q7w), once every eight weeks (q8w), once every nine weeks (q9w), once every ten weeks (q10w), once every eleven weeks (q11w), once every twelve weeks (q12w), or less frequently so long as a therapeutic response is achieved, are provided.

In certain embodiments involving the administration of a pharmaceutical composition comprising an anti-IL-4R antibody, once a week dosing of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every two weeks dosing (every two weeks is used interchangeably with every other week, bi-weekly or q2w) of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every three weeks dosing of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every four weeks dosing (monthly dosing) of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every five weeks dosing of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every six weeks dosing of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every eight weeks dosing of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every twelve weeks dosing of an amount of about 100 mg, about 200 mg, about 300 mg, about 400 mg, about 500 mg or about 600 mg can be employed. In certain exemplary embodiments, the route of administration is subcutaneous.

The term “week” or “weeks” refers to a period of (n×7 days)±3 days, e.g., (n×7 days)±2 days, (n×7 days)±1 day, or (n×7 days), wherein “n” designates the number of weeks, e.g., 1, 2, 3, 4, 5, 6, 8, 12 or more.

The terms “initial dose,” “secondary doses,” and “tertiary doses,” refer to the temporal sequence of administration of the IL-4R antagonist. Thus, the “initial dose” is the dose that is administered at the beginning of the treatment regimen (also referred to as the “baseline dose” or “loading dose”); the “secondary doses” are the doses that are administered after the initial dose; and the “tertiary doses” are the doses that are administered after the secondary doses. The initial, secondary, and tertiary doses may all contain the same amount of IL-4R antagonist or may differ from one another in terms of frequency of administration. In certain embodiments, however, the amount of IL-4R antagonist contained in the initial, secondary and/or tertiary doses varies from one another (e.g., adjusted up or down as appropriate) during treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”). In one embodiment, the maintenance dose may be lower than the loading dose. For example, one or more initial doses or loading doses of 600 mg or 400 mg of IL-4R antagonist may be administered followed by secondary doses or maintenance doses of about 75 mg to about 400 mg. In one embodiment, the secondary dose/maintenance dose may be equal to the initial dose/loading dose. For example, one or more initial doses/loading doses of 300 mg or 200 mg of IL-4R antagonist may be administered followed by secondary doses/maintenance doses of about 300 mg or about 200 mg, respectively. In one embodiment, a loading dose may be split, e.g., two or more doses administered at different time points, e.g., two loading doses wherein a second loading dose is administered two weeks after a first loading dose.

In certain embodiments, the initial dose is about 50 mg to about 600 mg of the IL-4R antagonist. In one embodiment, the initial dose is about 300 mg of the IL-4R antagonist. In another embodiment, the initial dose is about 200 mg of the IL-4R antagonist.

In certain embodiments, the secondary dose(s) are about 50 mg to about 600 mg of the IL-4R antagonist. In one embodiment, the secondary dose is about 300 mg of the IL-4R antagonist. In one embodiment, the secondary dose is about 200 mg of the IL-4R antagonist. In one embodiment, the secondary dose is about 300 mg of the IL-4R antagonist.

In certain embodiments, an initial dose is three times a secondary dose. In certain embodiments, an initial dose is two times a secondary dose. In certain embodiments, an initial dose is equal to a secondary dose. In an exemplary embodiment, the initial dose is about 300 mg and the maintenance dose(s) are about 300 mg. In another exemplary embodiment, the initial dose is about 200 mg and the secondary dose(s) are about 200 mg.

In some embodiments, the initial dose comprises 300 mg of the antibody or antigen-binding fragment thereof, and the one or more secondary doses comprises 300 mg of the antibody or antigen-binding fragment thereof administered every other week (every other week is used interchangeably with every two weeks, bi-weekly or q2w). In other embodiments, the subject is an adult, the initial dose comprises about 300 mg of the antibody or antigen-binding fragment thereof, and the one or more secondary doses comprises about 300 mg of the antibody or antigen-binding fragment thereof administered every other week (every other week is used interchangeably with every two weeks, bi-weekly or q2w).

In one exemplary embodiment, each secondary and/or tertiary dose is administered 1 to 14 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, or more) weeks after the immediately preceding dose. The phrase “the immediately preceding dose” means, in a sequence of multiple administrations, the dose of IL-4R antagonist that is administered to a patient prior to the administration of the very next dose in the sequence with no intervening doses.

The methods may include administering to a patient any number of secondary and/or tertiary doses of an IL-4R antagonist. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during treatment by a physician depending on the needs of the individual patient following clinical examination.

Methods comprising sequential administration of an IL-4R antagonist and a second therapeutic agent, to a patient to treat CRSsNP or an associated condition are provided. In some embodiments, the methods comprise administering one or more doses of an IL-4R antagonist followed by one or more doses (e.g., 2, 3, 4, 5, 6, 7, 8, or more) of a second therapeutic agent. For example, one or more doses of about 75 mg to about 600 mg of the IL-4R antagonist may be administered after which one or more doses (e.g., 2, 3, 4, 5, 6, 7, 8, or more) of a second therapeutic agent (e.g., a LAMA, a LABA, an ICS, an oral or systemic corticosteroid, or a combination thereof) may be administered to treat, alleviate, reduce or ameliorate one or more symptoms of CRSsNP. In some embodiments, the IL-4R antagonist is administered at one or more doses (e.g., 2, 3, 4, 5, 6, 7, 8, or more) resulting in an improvement in one or more CRSsNP-associated parameters followed by the administration of a second therapeutic agent to prevent recurrence of at least one symptom of CRSsNP. Alternative embodiments pertain to concomitant administration of an IL-4R antagonist and a second therapeutic agent. For example, one or more doses (e.g., 2, 3, 4, 5, 6, 7, 8, or more) of an IL-4R antagonist are administered and a second therapeutic agent is administered at a separate dosage at a similar or different frequency relative to the IL-4R antagonist. In some embodiments, the second therapeutic agent is administered before, after or concurrently with the IL-4R antagonist.

In certain embodiments, the IL-4R antagonist is administered every other week for 12 weeks, 14 weeks, 16 weeks, 18 weeks, 20 weeks, 22 weeks, 24 weeks, 26 weeks, 28 weeks, 30 weeks, 32 weeks, 34 weeks, 36 weeks, 38 weeks, 40 weeks, 42 weeks, 44 weeks, 46 weeks, 48 weeks, 50 weeks, 52 weeks, or more. In other embodiments, the IL-4R antagonist is administered every four weeks for 12 weeks, 16 weeks, 20 weeks, 24 weeks, 28 weeks, 32 weeks, 36 weeks, 40 weeks, 44 weeks, 48 weeks, 52 weeks or more. In specific embodiments, the IL-4R antagonist is administered for at least 52 weeks.

In certain embodiments, a kit comprising a dosage form of an antibody, or an antigen-binding fragment thereof, that specifically binds interleukin-4 receptor (IL-4R), wherein the antibody or antigen-binding fragment thereof comprises three heavy chain CDR sequences comprising SEQ Id NOs: 3, 4, and 5, respectively, and three light chain CDR sequences comprising SEQ Id NOs: 6, 7, and 8, respectively, for the treatment of CRSsNP is provided. In certain embodiments, the antibody or antigen-binding fragment thereof comprises a heavy chain variable region (HCVR) sequence of SEQ ID NO: 1 and a light chain variable region (LCVR) sequence of SEQ ID NO: 2. In certain embodiments, the antibody is dupilumab.

The kit can comprise a label or package insert, wherein the label or package insert comprises instructions for administering the dosage form for the treatment of CRSsNP. The instructions can recite a dosing regimen described further herein for the treatment of CRSsNP.

### Treatment Populations

The methods featured in the present disclosure include administering to a subject in need thereof a therapeutic composition comprising an IL-4R antagonist. As used herein, the expression “a subject in need thereof” means a human or non-human animal that exhibits one or more CRSsNP symptoms, Type 2 mediated inflammatory response, characteristic CT findings, sinus opacification on computerized tomography (CT) scan or any combination thereof, or a subject who has been diagnosed with one of these conditions. In some embodiments, a subject in need thereof is one who is at least 12 years of age. In other embodiments, the subject is an adolescent (between the ages of 12-17 years). In other embodiments, the subject is an adult (18 years or older). In some embodiments, a “subject in need thereof” is an adult or an adolescent. In some embodiments, a “subject in need thereof” may be a subject with blood eosinophils≥300 cells/mm³, 200-299 cells/mm³, or <300 cells/mm³. In some embodiments, the subjects are stratified into the following groups: a blood eosinophil count≥300 cells/μL (Eos) or 300-499 cells/μL or ≥500 cells/μL, a blood eosinophil count of 200 to 299 cells/μL, or a blood eosinophil count<300 cells/μL, and are administered an anti-IL-4R antibody or antigen binding fragment thereof at a dose or dosing regimen based upon the eosinophil level.

A subject in need thereof may further have been diagnosed based on one or more of the following: (a) 22-item Sino Nasal Outcome Test (SNOT-22) score; (b) subject-assessed nasal congestion/obstruction, anterior rhinorrhea, posterior rhinorrhea, facial pain or pressure and loss of sense of smell; (c) Visual Analog Score (VAS) to assess patient-rated rhinosinusitis symptom severity; (e) six-item Asthma Control Questionnaire (ACQ6) score in patients with asthma; (g) Nasal Peak Inspiratory Flow (NPIF); (h) smell test (University of Pennsylvania Smell Identification Test (UPSIT); (i) physiological parameters, such as measured by nasal endoscopy and CT scan; (j) Lund-Mackay Score; and (k) Three Dimensional volumetric measurement of the maxillary sinus.

For example, in some embodiments, a “subject in need thereof” is a human patient with chronic symptoms of CRSsNP, which are the presence of CRSsNP symptoms.

In some embodiments, a “subject in need thereof” is a subject with elevated levels of CRSsNP-associated biomarkers. In some embodiments, a “subject in need thereof” is a subject with elevated levels of one or any combination of IgE, TARC, eotaxin-3, periostin, IL-5, tryptase, eosinophil cationic protein, secreted P-glycoprotein, eosinophils.

In certain embodiments, a “subject in need thereof” is a human patient with a SNOT-22 score of greater than about 7, greater than about 10, greater than about 15, greater than about 20, greater than about 25, greater than about 30, greater than about 35, greater than about 40, greater than about 45, or greater than about 50. A “subject in need thereof” may also be a human patient who exhibits a Lund-Mackay score of greater than about 4, greater than about 5, greater than about 6, greater than about 7, greater than about 8, greater than about 9, greater than about 10, greater than about 11, greater than about 12, or greater than about 13. A “subject in need thereof” may also be a human patient who exhibits a sTSS score of greater than about 4, greater than about 5, greater than about 6, or greater than about 7.

In a related embodiment, a “subject in need thereof” may be a subject who, prior to receiving an IL-4R antagonist underwent or plans to undergo endoscopic nasal surgery for CRSsNP. In a related embodiment, a “subject in need thereof” may be a subject who, prior to receiving an IL-4R antagonist, has been prescribed or is currently taking another medication, “a background therapy.” In another related embodiment, a “subject in need thereof” may be a subject who, prior to receiving an IL-4R antagonist, has been prescribed or is currently taking another medication, “a background therapy following sinonasal surgery such as endoscopic sinus surgery (ESS). The background therapy can be, for example, an oral or topical corticosteroid. In some embodiments, a “subject in need thereof” is an asthma patient who prior to receiving an IL-4R antagonist, has been prescribed or is currently taking oral or topical corticosteroid particular following ESS. In some embodiments, the “subject in need thereof” continues the background therapy after the subject receives the IL-4R antagonist, and in other embodiments, the subject in need thereof stops receiving the background therapy (e.g., at once or gradually) before receiving the IL-4R antagonist.

### Methods for Assessing CRSsNP-Associated Parameters

Methods for assessing one or more CRSsNP symptoms in a subject in need thereof, caused by administration of a pharmaceutical composition comprising an IL-4R antagonist, are provided. In some embodiments, administration of the pharmaceutical composition comprising an IL-4R antagonist results in the reduction or improvement of sinus opacification and total symptom score (sTSS). In some embodiments, methods of assessing efficacy are by measuring changes in baseline in the LMK CT scan score and sTSS at week 24, which will provide objective imaging-based measurement of benefit and evidence of symptomatic improvement in cardinal clinical manifestations of CRSsNP patients. In some embodiments, methods of assessing efficacy are by measuring changes in baseline in the LMK CT scan score and sTSS at week 52 which will provide evidence of the durability of response and the long-term safety of the anti-IL-4R antibody.

In some embodiments, a reduction in the incidence of CRSsNP symptoms or an improvement in an CRSsNP-associated parameters may correlate with an improvement in one or more pharmacodynamic CRSsNP-associated parameters; however, such a correlation is not necessarily observed in all cases. Examples of “pharmacodynamic CRSsNP-associated parameters” include, for example, the following: (a) biomarker expression levels and (b) serum protein and RNA analysis. An “improvement in a pharmacodynamic CRSsNP-associated parameter” means, for example, a decrease from baseline of one or more biomarkers, such as pulmonary and activation-regulated chemokine (PARC), eotaxin-3, fibrinogen, IgE, blood or sputum eosinophils, blood or sputum neutrophils, or FeNO. As used herein, the term “baseline,” regarding a pharmacodynamic CRSsNP-associated parameter, means the numerical value of the pharmacodynamic CRSsNP-associated parameter for a patient prior to or at the time of administration of a pharmaceutical composition described herein.

To assess a pharmacodynamic CRSsNP-associated parameter, the parameter is quantified at baseline and at a time point after administration of the pharmaceutical composition. For example, a pharmacodynamic CRSsNP-associated parameter may be measured at about day 1, about day 2, about day 3, day 4, about day 5, about day 6, about day 7, about day 8, about day 9, about day 10, about day 11, about day 12, about day 14, or at about week 3, about week 4, about week 5, about week 6, about week 7, about week 8, about week 9, about week 10, about week 11, about week 12, about week 13, about week 14, about week 15, about week 16, about week 17, about week 18, about week 19, about week 20, about week 21, about week 22, about week 23, about week 24, or week 52, after the initial treatment with the pharmaceutical composition. The difference between the value of the parameter at a particular time point following initiation of treatment and the value of the parameter at baseline is used to establish whether there has been change, such as an “improvement,” in the pharmacodynamic CRSsNP associated parameter (e.g., an increase or decrease depending on the specific parameter being measured).

In certain embodiments, administration of an IL-4R antagonist to a patient causes a change, such as a decrease or increase, in expression of a particular biomarker. CRSsNP-associated biomarkers include, but are not limited to IgE, TARC, eotaxin-3, periostin, IL-5, tryptase, eosinophil cationic protein, secreted P-glycoprotein, and eosinophils. For example, administration of an IL-4R antagonist to a CRSsNP patient can cause a decrease in IgE mediated inflammatory response to fungal hyphae. The decrease can be detected at about week 1, about week 2, about week 3, about week 4, about week 5, week 12, week 16, week 24, or week 52 following administration of the IL-4R antagonist. Biomarker expression can be assayed by methods known in the art. For example, protein levels can be measured by ELISA (enzyme linked immunosorbent assay). RNA levels can be measured, for example, by reverse transcription coupled to polymerase chain reaction (RT-PCR).

Biomarker expression, as discussed above, can be assayed by detection of protein or RNA in serum. The serum samples can also be used to monitor additional protein or RNA biomarkers related to response to treatment with an IL-4R antagonist or IL-4/IL-13 signaling (e.g., by measuring soluble IL-4Rα, IL-4, IL-13, etc.). In some embodiments, RNA samples are used to determine RNA levels (non-genetic analysis), e.g., RNA levels of biomarkers; and in other embodiments, RNA samples are used for transcriptome sequencing (e.g., genetic analysis).

### Formulations

In some embodiments, the antibody or antigen binding fragment thereof is formulated in a composition comprising: i) about 150 mg/mL of antibody or an antigen-binding fragment thereof that specifically binds to IL-4R, ii) about 20 mM histidine, iii) about 12.5 mM acetate, iv) about 5% (w/v) sucrose, v) about 25 mM arginine hydrochloride, vi) about 0.2% (w/v) polysorbate 80, wherein the pH of the formulation is about 5.9, and wherein the viscosity of the formulation is about 8.5 cPoise.

In alternative embodiments, the antibody or antigen binding fragment thereof is formulated in a composition comprising: i) about 175 mg/mL of antibody or an antigen-binding fragment thereof that specifically binds to IL-4R, ii) about 20 mM histidine, iii) about 12.5 mM acetate, iv) about 5% (w/v) sucrose, v) about 50 mM arginine hydrochloride, and vi) about 0.2% (w/v) polysorbate 80, wherein the pH of the formulation is about 5.9, and wherein the viscosity of the formulation is about 8.5 cPoise.

In specific embodiments, the antibody or antigen-binding fragment thereof comprises an HCVR comprising the amino acid sequence of SEQ ID NO: 1 and an LCVR comprising the amino acid sequence of SEQ ID NO: 2.

In specific embodiments, the antibody comprises dupilumab. Unless otherwise specified, the term “dupilumab” also includes any biosimilars thereof.

Suitable stabilized formulations are also set forth in U.S. Pat. No. 8,945,559, which is incorporated herein by reference in its entirety for all purposes.

The present disclosure is further illustrated by the following example which should not be construed as further limiting. The contents of the figures, tables and all references, patents and published patent applications cited throughout this application are expressly incorporated herein by reference for all purposes.

Furthermore, in accordance with the present disclosure there may be employed molecular biology, microbiology, and recombinant DNA techniques within the skill of the art. Such techniques are explained fully in the literature. See, e.g., Green & Sambrook, Molecular Cloning: A Laboratory Manual, Fourth Edition (2012) Cold Spring Harbor Laboratory Press, Cold Spring Harbor, New York; DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription and Translation [B. D. Hames & S. J. Higgins, eds. (1984)]; Animal Cell Culture [R. I. Freshney, ed. (1986)]; Immobilized Cells and Enzymes [IRL Press, (1986)]; B. Perbal, A Practical Guide to Molecular Cloning (1984); F. M. Ausubel et al. (eds.), Current Protocols in Molecular Biology, John Wiley & Sons, Inc. (1994).

In some embodiments, a subject has CRSsNP, and the initial dose comprises 300 mg of the antibody or antigen-binding fragment thereof, the one or more subsequent doses comprises 300 mg of the antibody or antigen-binding fragment thereof administered every other week.

In some embodiments, a subject has CRSsNP with Type 2 inflammation, and the initial dose comprises 300 mg of the antibody or antigen-binding fragment thereof, and the one or more subsequent doses comprises 300 mg of the antibody or antigen-binding fragment thereof administered every other week.

In some embodiments, a subject has uncontrolled CRSsNP with Type 2 inflammation, and the initial dose comprises 300 mg of the antibody or antigen-binding fragment thereof, and the one or more subsequent doses comprises 300 mg of the antibody or antigen-binding fragment thereof administered every other week.

In some embodiments, a subject has uncontrolled CRSsNP with Type 2 inflammation, and the initial dose comprises 300 mg of the antibody or antigen-binding fragment thereof, and the one or more subsequent doses comprises 300 mg of the antibody or antigen-binding fragment thereof administered every other week, wherein the subject is 18 years or older, or the subject is 12-17 years and is greater than or equal to 60 kg.

In some embodiments, a subject has uncontrolled CRSsNP with Type 2 inflammation, and the initial dose comprises 200 mg of the antibody or antigen-binding fragment thereof, and the one or more subsequent doses comprises 200 mg of the antibody or antigen-binding fragment thereof administered every other week, wherein the subject is 12-17 years and is greater than or equal to 30 kg and less than 60 kg.

### EXAMPLE

The following examples are put forth to provide those of ordinary skill in the art with a complete disclosure and description of how to make and use the methods and compositions featured in the disclosure and is not intended to limit the scope of what the inventors regard as their disclosure. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

The exemplary IL-4R antagonist used in the following Example is the human anti-IL-4R antibody named dupilumab (also referred to herein as “mAb1” or DUPIXENT®).

### Example 1. A Randomized, Double-Blind, Placebo-Controlled, 2-Part Study Evaluating the Efficacy and Safety of Dupilumab in Patients with Uncontrolled CRSsNP

### Study Objectives and Overview

This is a Phase 2/3 multicenter, 2-part study to evaluate the efficacy and safety of dupilumab compared to placebo, in participants with uncontrolled CRSsNP. Part A is a randomized, double-blind, placebo-controlled study in adult participants regardless of screening eosinophil count (52 weeks) to evaluate the treatment effect of dupilumab on sinus opacification as assessed by the LMK sinus CT scan total score and on CRSsNP symptoms as measured by the sTSS, and to provide long-term data on durability of response to dupilumab and safety. Part B is a randomized, double-blind, placebo-controlled study, in adult and adolescent participants (24 weeks) to confirm and provide duplicate evidence of the effectiveness of dupilumab in participants with CRSsNP with evidence of elevated blood eosinophil count.

### Objectives

### Primary Objectives for Part A

To evaluate the efficacy of dupilumab as assessed by the reduction at week 24 in sinus opacification on computerized tomography (CT) scan and sinus total symptom score (sTSS) compared to placebo.

### Secondary Objective for Part A

To evaluate the efficacy of dupilumab as assessed by the reduction at week 52 in sinus opacification on CT scan and sinus total symptom score (sTSS) compared to placebo. To evaluate the efficacy of dupilumab in improving CRSsNP symptoms at Weeks 24 and 52 compared to placebo.

To evaluate the effect of dupilumab on health-related quality of life at weeks 24 and 52 (HRQoL) compared to placebo.

To evaluate the effect of dupilumab on CRSsNP overall disease severity at weeks 24 and 52 compared to placebo.

To evaluate the effect of dupilumab at weeks 24 and 52 in the subgroups of participants with comorbid asthma compared to placebo.

To evaluate the ability of dupilumab to reduce the incidence of participants with CRSsNP worsening/acute sinusitis who require treatment with antibiotics, SCS or sinus surgery compared to placebo.

To evaluate the effects of dupilumab on transcriptomic signatures associated with CRSsNP and type-2 inflammation.

To evaluate the effect of dupilumab in the subgroup of participants with screening blood eosinophils count≥300 cells/mm³ compared to placebo.

To evaluate the safety and tolerability of dupilumab in CRSsNP patients compared to placebo.

To evaluate the pharmacokinetics (PK) of dupilumab in CRSsNP patients compared to placebo.

Assessment of immunogenicity to dupilumab over time compared to placebo.

### Tertiary/Exploratory Objectives for Part A

To evaluate the effect of dupilumab on overall health status, global impression of disease severity/change, and health care resource use/productivity compared to placebo.

To characterize the effect of dupilumab in CRSsNP patients by evaluating blood, urine, and nasal biomarkers compared to placebo.

To characterize the effect of dupilumab on gene expression in nasal brushings and blood in consenting participants.

To investigate genetic factors that may predict dupilumab safety or efficacy.

### Primary Objectives for Part B

To evaluate the efficacy of dupilumab as assessed by the reduction at week 24 in sinus opacification on computerized tomography (CT) scan and sinus total symptom score (sTSS) compared to placebo.

### Secondary Objectives for Part B

To evaluate the efficacy of dupilumab in improving CRSsNP symptoms at week 24 compared to placebo in patients.

To evaluate the effect of dupilumab on health-related quality of life (HRQoL) compared to placebo.

To evaluate the effect of dupilumab on CRSsNP overall disease severity compared to placebo.

To evaluate the effect of dupilumab in the subgroups of participants with comorbid asthma compared to placebo.

To evaluate the effect of dupilumab to reduce the incidence of participants with CRSsNP worsening/acute sinusitis who require treatment with antibiotics, SCS, or sinus surgery compared to placebo.

To evaluate the effects of dupilumab transcriptomic signatures associated with CRSsNP and type-2 inflammation.

To evaluate the safety and tolerability of dupilumab in CRSsNP patients compared to placebo.

To evaluate the pharmacokinetics (PK) of dupilumab in CRSsNP patients compared to placebo.

Assessment of immunogenicity to dupilumab over time compared to placebo.

### Tertiary/Exploratory Objectives for Part B

To evaluate the effect of dupilumab on overall health status, global impression of disease severity/change, and health care resource use/productivity compared to placebo.

To characterize the effect of dupilumab in CRSsNP patients by evaluating blood, urine, and nasal biomarkers compared to placebo.

To characterize the effect of dupilumab on gene expression in nasal brushings and blood in consenting participants.

To investigate genetic factors that may predict dupilumab safety or efficacy.

### Endpoints

### Primary Part A

Change from baseline to week 24 in opacification of sinuses assessed by CT scan using the Lund-Mackay (LMK) score.

Change from baseline to week 24 in the sTSS.* (*Composite severity score consisting of nasal congestion, anterior/posterior rhinorrhea, facial pain/pressure items of the CRSsNP sinonasal symptom e-diary).

### Secondary Part A

Change from baseline to week 52 in opacification of sinuses assessed by CT scan using the LMK score.

Change from baseline to week 52 in sTSS.

Change from baseline to weeks 24 and 52 in:
Nasal congestion (NC) symptom severity score using the daily CRSsNP sinonasal symptom e-diary
Anterior/posterior rhinorrhea severity score using the daily CRSsNP sinonasal symptom e-diary.
Facial pain/pressure severity score using the daily CRSsNP sinonasal symptom e-diary.
Loss of smell symptom severity score using the daily CRSsNP sinonasal symptom e-diary.
University of Pennsylvania smell identification test (UPSIT).

Change from baseline to weeks 24 and 52 in Sino-Nasal Outcome Test-22 item (SNOT-22).

Change from baseline to weeks 24 and 52 in rhinosinusitis severity visual analog scale (VAS).

Change from baseline to weeks 24 and 52 in
Forced expiratory volume (FEV1) in CRSsNP with asthma population.
Asthma control questionnaire-6 items (ACQ-6) in CRSsNP with asthma population.
Opacification of sinuses assessed by CT scan using the LMK score in CRSsNP with asthma population.
sTSS using the CRSsNP e-diary assessment in CRSsNP with asthma population.

Proportion of participants with CRSsNP worsening/acute sinusitis* during the planned study treatment period. (*worsening of any CRS symptoms that requires initiation of rescue therapy).

Annualized rate of SCS course* during the planned study treatment period. (*A course of SCS is considered continuous if treatment is separated by less than 7 days).

Normalized Enrichment Score (NES) for the relative change from baseline to Weeks 24 and 52 in the type-2 inflammation transcriptome signature.

Change from baseline to weeks 24 and 52 in opacification of sinuses assessed by CT scan using LMK score in the screening blood eosinophil count≥300 cells/mm³ population.

Change from baseline to weeks 24 and 52 in sTSS using the screening blood eosinophil count≥300 cells/mm³ population.

Evaluation of other secondary endpoints listed above in the screening blood eosinophil count≥300 cells/mm3 population.

Incidence of treatment-emergent adverse events (TEAEs), of treatment-emergent serious AEs (TESAEs), and TEAEs leading to treatment discontinuation, abnormal laboratory values, and vital signs.

Dupilumab concentration in serum.

Assessment of immunogenicity to dupilumab as determined by the incidence, titer, and neutralizing antibody (NAb) status of treatment-emergent anti-drug antibody (ADA) response over time compared to placebo.

### Tertiary/Exploratory Part A

Change from baseline to weeks 24 and 52 in the EQ-5D-5L.

Cumulative number of health care resource utilization.

Cumulative number of missed days of school/work.

Change from baseline to weeks 24 and 52 in Patient Global Impression of Severity (PGIS) of CRSsNP.

Patient Global Impression of Change (PGIC) in CRSsNP at Weeks 24 and 52.

Change from baseline to weeks 24 and 52 in headache severity score using the symptom diary.

Pharmacodynamic biomarkers in blood, nasal secretion and urine.

RNA analysis of sinonasal brushings in all participants.

DNA and whole blood RNA (optional genomics for adults).

### Primary Part B

Change from baseline to week 24 in opacification of sinuses assessed by CT scan using the Lund-Mackay (LMK) score.

Change from baseline to week 24 in sTSS.* (*Composite severity score consisting of nasal congestion, anterior/posterior rhinorrhea, facial pain/pressure items of the CRSsNP sinonasal symptom e-diary.

### Secondary Part B

Change from baseline to week 24 in:
NC symptom severity score using the CRSsNP e-diary
Anterior/posterior rhinorrhea severity score using the daily e-diary
Facial pain/pressure severity score using the daily e-diary
Loss of smell symptom severity score using the e-diary
University of Pennsylvania smell identification test (UPSIT)

Change from baseline to week 24 in Sino-Nasal Outcome Test-22 item (SNOT-22).

Change from baseline to week 24 in rhinosinusitis severity visual analog scale (VAS).

Change from baseline to week 24 in FEV1 in CRSsNP with asthma population.

Change from baseline to week 24 in asthma control questionnaire-6 items (ACQ-6) in CRSsNP with asthma population.

Change from baseline to week 24 in opacification of sinuses assessed by CT scan using the LMK score in CRSsNP with asthma population.

Change from baseline to week 24 in sTSS using the CRSsNP e-diary assessment in CRSsNP with asthma population.

Proportion of participants with CRSsNP worsening/acute sinusitis* during the planned study treatment period. *Worsening of any CRS symptoms that requires initiation of rescue therapy.

Annualized rate of SCS course* during the planned study treatment period

*A course of SCS is considered continuous if treatment is separated by less than 7 days.

Normalized Enrichment Score (NES) for the relative change from baseline to Weeks 24 in the type-2 inflammation transcriptome signature.

Incidence of treatment-emergent adverse events (TEAEs), of treatment-emergent serious AEs (TESAEs), and TEAEs leading to treatment discontinuation, abnormal laboratory values, and vital signs.

Dupilumab concentration in serum.

Assessment of immunogenicity to dupilumab as determined by the incidence, titer, and neutralizing antibody (NAb) status of treatment-emergent anti-drug antibody (ADA) response over time compared to placebo.

### Tertiary/Exploratory Part B

Change from baseline to week 24 in the EQ-5D-5L.

Cumulative number of health care resource utilization.

Cumulative number of missed days of school/work.

Change from baseline to week 24 in Patient Global Impression of Severity (PGIS) of CRSsNP.

Patient Global Impression of Change (PGIC) in CRSsNP at Week 24

Change from baseline to week 24 in headache severity score using the symptom diary.

Pharmacodynamic biomarkers in blood, nasal secretion, and urine.

RNA analysis of sinonasal brushings in all participants.

DNA and whole blood RNA (optional genomics for adults).

### Overall Design

Part A: All participants in Part A (adults only) will enter a screening period (2 to 4 weeks), with a 52-week treatment period, followed by post-treatment follow-up (12 weeks).

Approximately 140 adult participants will be randomized 1:1 (approximately 70 participants per arm) into 2 treatment groups as follows:
Arm A: Dupilumab 300 mg q2w,
Arm B: Matching placebo.

Randomization will be stratified by screening blood eosinophil count (?300 cells/mm3 or <300 cells/mm3), background INCS use (yes or no) and region.

To ensure enrollment according to the intended distribution of screening blood eosinophil count, alerts will be built into the IRT to control the number of participants in each stratification group as follows:
≥300 cells/mm3: approximately 50 participants per arm,
<300 cells/mm3: approximately 20 participants per arm.

Part B: All participants in Part B (adults and adolescents) will enter a screening period

(2 to 4 weeks), with a 24-week treatment period, followed by post-treatment follow-up (12 weeks).

Approximately 100 participants (adults and adolescents) will be randomized 1:1 (50 participants per arm) into 2 treatment groups as follows:
Arm A: Dupilumab 300 mg q2w for (for all adults and those adolescents≥60 kg at screening) or dupilumab 200 mg q2w (for adolescents≥30 kg and <60 kg at screening),
Arm B: Matching placebo.

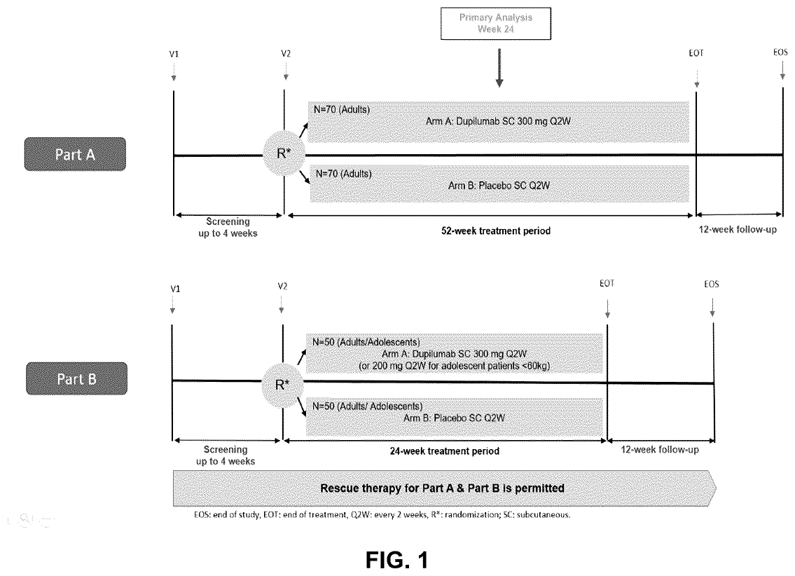

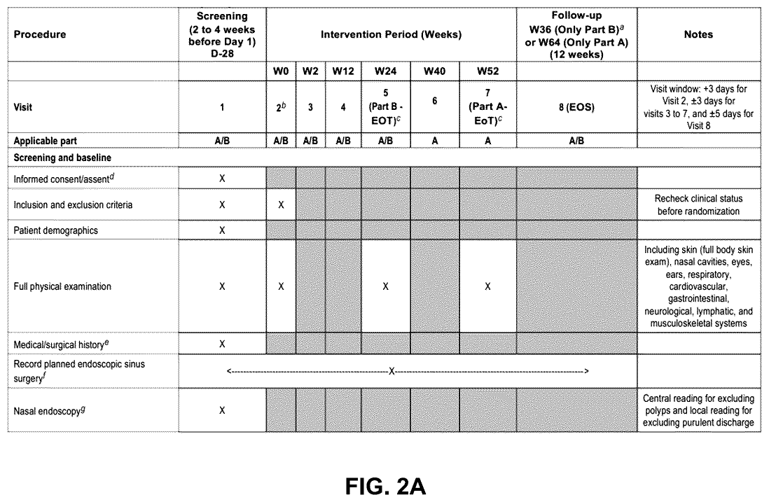

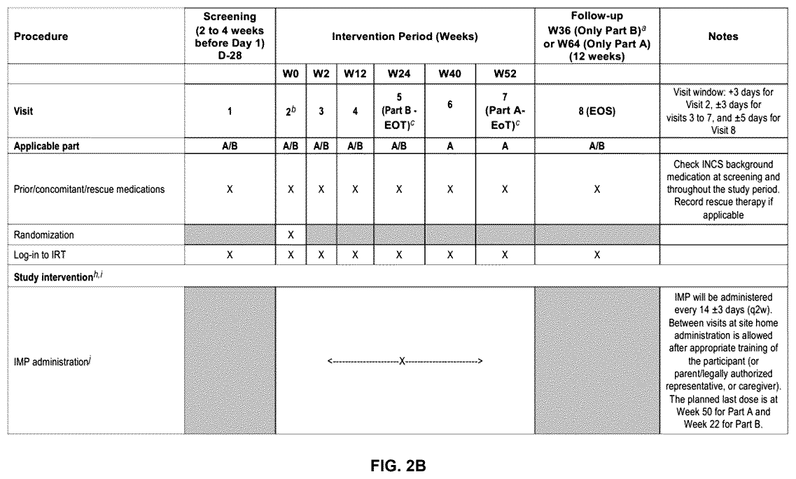

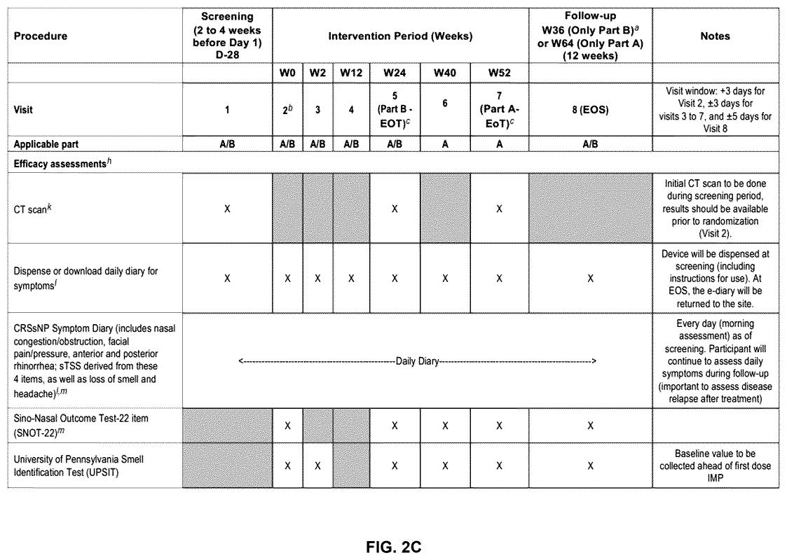

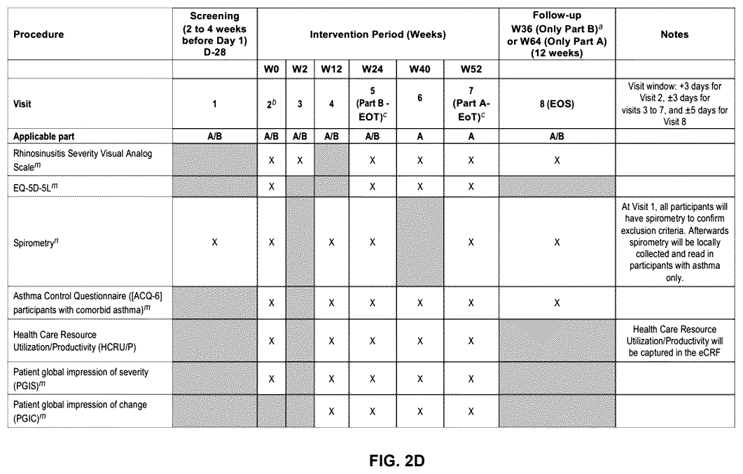

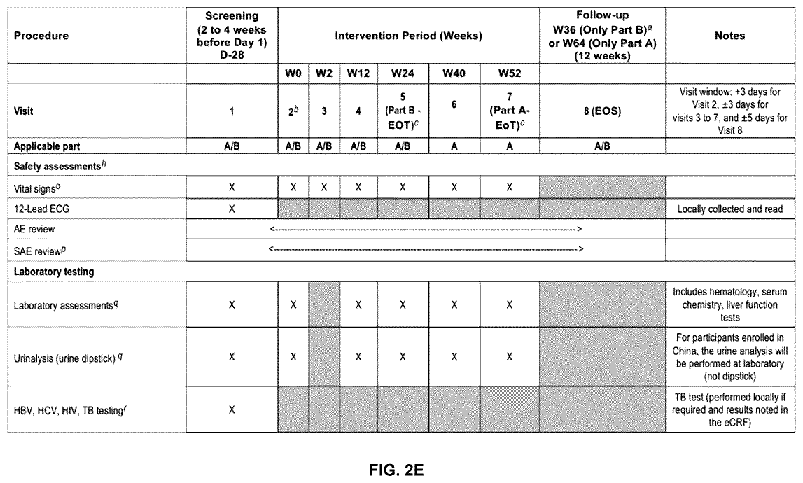

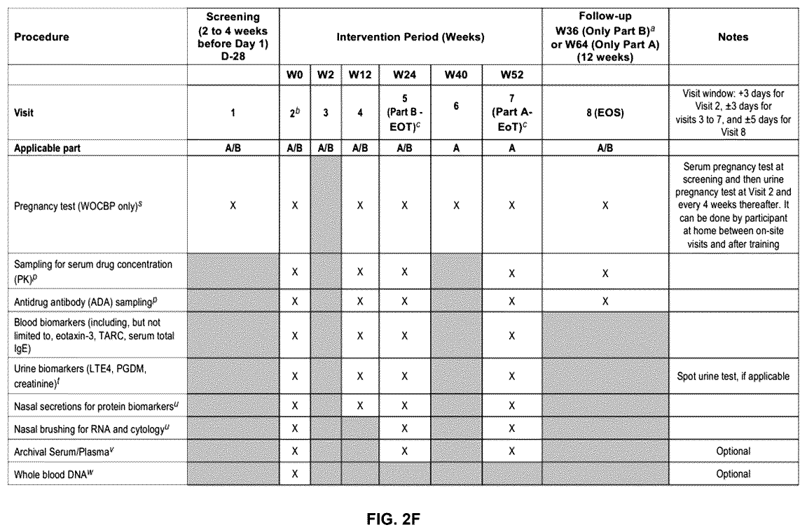

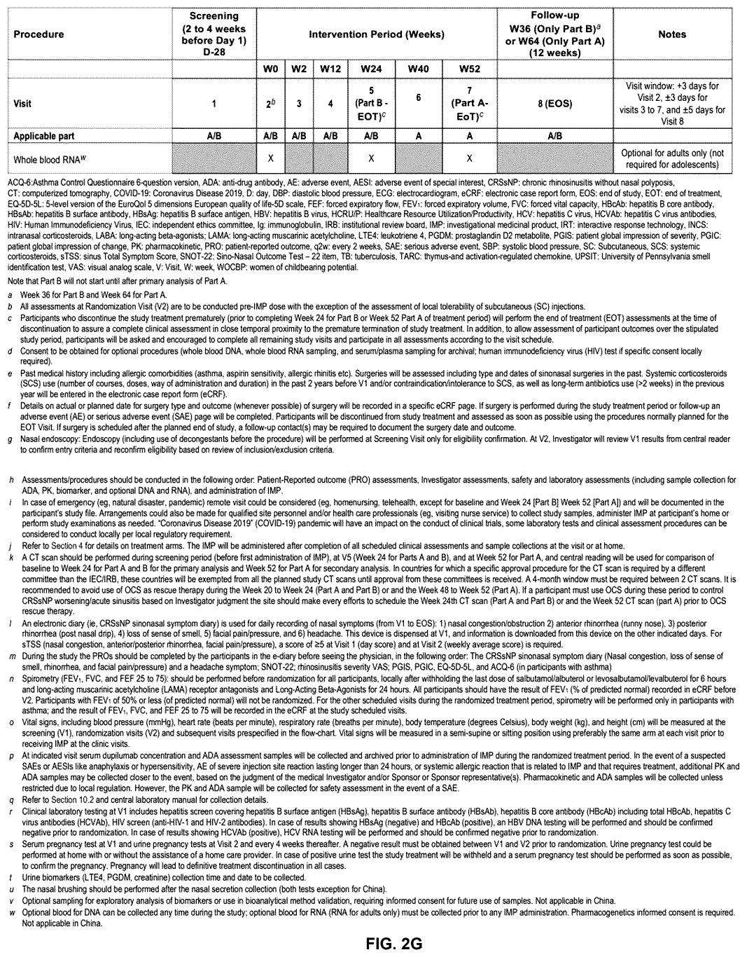

A schematic of the study protocol is provided in FIG. 1.

### Inclusion Criteria

Participants are eligible to be included in the study only if all of the following criteria apply:
Part A only: Participant must be at least 18 years of age at the time of signing the informed consent form (ICF).
Part B only: Participant must be at least adolescents in the country of the investigational site) at the time of signing the ICF.

### Type of Participant and Disease Characteristics

Participants must have bilateral inflammation of paranasal sinuses in CT scan with LMK≥8 and bilateral ethmoid opacification before randomization.

Participants must have ongoing symptoms of loss of smell and rhinorrhea (anterior/posterior) of any severity, with or without facial pain/pressure for at least 12 consecutive weeks by Visit 1.

Participants must have ongoing symptoms of NC/obstruction at least 12 consecutive weeks before Visit 1 and a NC score of ≥2 at Visit 1 (day score) and Visit 2 (weekly average score).

Participants must have sTSS (NC, rhinorrhea, facial pain/pressure)≥5 at Visit 1 (day score) and Visit 2 (weekly average score).

Participants must have at least one of the 2 following features:
a) Prior sinonasal surgery (see note at end of section 5.2 for definitions of sinonasal surgery) for CRS,
b) Treatment with SCS therapy for CRS as defined by any dose and duration within the prior 2 years before screening (Visit 1) or intolerance/contraindication to SCS.

Part A only: No restrictions on eosinophil count.

Part B only: participants who have a blood eosinophil count≥300 cells/mm³ at Screening.

Body weight≥30 kg.

Male or female.

Contraceptive use by women should be consistent with local regulations regarding the methods of contraception for those participating in clinical studies.
A female participant is eligible to participate if she is not pregnant or breastfeeding, and at least 1 of the following conditions applies:
   Is not a woman of childbearing potential (WOCBP).
   OR
   Is a WOCBP and agrees to use a contraceptive method that is highly effective, with a failure rate of <1%, as described in Appendix of the protocol during the study (at
   a minimum until 12 weeks after the last dose of study intervention).
A WOCBP must have a negative highly sensitive pregnancy test (urine or serum as required by local regulations) on Day 1 before the first dose of study intervention,
If a urine test cannot be confirmed as negative (e.g., an ambiguous result), a serum pregnancy test is required. In such cases, the participant must be excluded from participation if the serum pregnancy result is positive,
The Investigator is responsible for review of medical history, menstrual history, and recent sexual activity to decrease the risk for inclusion of a woman with an early undetected pregnancy.

### Exclusion Criteria

Participants are excluded from the study if any of the following criteria apply:

### Medical Conditions

Participants with conditions/concomitant diseases making them non-evaluable at Visit 1 or for the primary efficacy endpoint such as:
a) Participants with nasal polyposis observed during nasal endoscopy at Visit 1,
b) Participants with past history of nasal polyposis,
c) Nasal septal deviation that would cause complete occlusion of at least one nostril,
d) Acute sinusitis or purulent drainage or nasal infection, or upper respiratory infection at Visit 1 or Visit 2,
e) Ongoing rhinitis medicamentosa,
f) Eosinophilic granulomatous polyangiitis (Churg-Strauss syndrome), granulomatosis with polyangiitis (Wegener's granulomatosis), microscopic polyangiitis, Young's syndrome, Kartagener's syndrome or other dyskinetic ciliary syndromes, cystic fibrosis.

Participants with nasal cavity malignant tumor and benign tumors (eg, papilloma, hemangioma).

Participants with Forced expiratory volume (FEV1)≤50% of predicted normal at Visit 1.

Radiologic suspicion or confirmed invasive or expansive fungal rhinosinusitis.

Diagnosed with, suspected of, or at high risk of endoparasitic infection, and/or use of antiparasitic drug within 2 weeks before the screening visit (visit 1) or during the screening period.

History of human immunodeficiency virus (HIV) infection or positive HIV screen (anti-HIV-1 and HIV-2 antibodies) serology at the screening visit (visit 1).

Severe concomitant illness(es) that, in the Investigator's judgment, would adversely affect participation in the study. Examples include, but are not limited to, participants with short life expectancy, participants with uncontrolled diabetes (hemoglobin A1c≥9%), participants with cardiovascular conditions (e.g., Class III or IV cardiac failure according to the New York Heart Association classification), severe renal conditions (e.g., participants on dialysis), hepato-biliary conditions (e.g., Child-Pugh class B or C), neurological conditions (e.g., demyelinating diseases), active major autoimmune diseases (e.g., lupus, inflammatory bowel disease, rheumatoid arthritis), and other severe endocrinological, gastrointestinal, metabolic, pulmonary, or lymphatic diseases. The specific justification for participants excluded under this criterion will be noted in study documents (chart notes, electronic case report forms [eCRFs], etc.).

Known or suspected immunodeficiency, including history of invasive opportunistic infections e.g., tuberculosis (TB), histoplasmosis, listeriosis, coccidioidomycosis, pneumocystosis, and aspergillosis), despite infection resolution, or otherwise recurrent infections of abnormal frequency or prolonged duration suggesting an immune-compromised status, as judged by the Investigator.

Participants with active TB, non-tuberculous mycobacterial infection, or a history of incompletely treated TB will be excluded from the study unless it is well documented by a specialist that the participant has been adequately treated and can now start treatment with a biologic agent, in the medical judgment of the Investigator and/or infectious disease specialist. Tuberculosis testing would be performed on a country by country basis according to local guidelines if required by regulatory authorities or ethics boards.

Active chronic or acute infection requiring treatment with systemic antibiotics, antivirals, or antifungals within 2 weeks before the screening visit (visit 1) or during the screening period.

History of malignancy within 5 years before visit 1, except completely treated in situ carcinoma of the cervix, and completely treated and resolved nonmetastatic squamous or basal cell carcinoma of the skin.

Known or suspected alcohol and/or drug abuse.

History of systemic hypersensitivity or anaphylaxis to dupilumab or any of its excipients.

Planned major surgical procedure during the patient's participation in this study.

Participant with any other medical or psychological condition including relevant laboratory or electrocardiogram abnormalities at screening that, in the opinion of the Investigator, suggest a new and/or insufficiently understood disease, may present an unreasonable risk to the study participant as a result of his/her participation in this clinical trial, may make patient's participation unreliable, or may interfere with study assessments. The specific justification for participants excluded under this criterion will be noted in study documents (chart notes, eCRF, etc.).

### Prior/Concomitant Therapy

Participation in prior dupilumab clinical study or have been treated with commercially available dupilumab within 12 months or who discontinued dupilumab use due to adverse event.

Participants who have taken:
Biologic therapy/systemic immunosuppressant such as methotrexate, cyclosporine, mycophenolate, tacrolimus, etc to treat inflammatory disease (including CRSsNP) or autoimmune disease (eg, rheumatoid arthritis, inflammatory bowel disease, primary biliary cirrhosis, systemic lupus erythematosus, multiple sclerosis) within 4 weeks before visit 1 or 5 half-lives, whichever is longer,
Any investigational monoclonal antibody (mAb) within 5 half-lives or within 6 months before visit 1 if the half-life is unknown,
Anti-immunoglobulin E therapy (omalizumab) within 4 months prior to visit 1.

Treatment with a live (attenuated) vaccine within 4 weeks before the screening visit (visit 1).
NOTE: For participants who have vaccination with live, attenuated vaccines planned during the study (based on national vaccination schedule/local guidelines), it will be determined, after consultation with a physician, whether the administration of vaccine can be postponed until after the end of study (EOS), or preponed to before the start of the study without compromising the health of the participant:
Participants for whom administration of live (attenuated) vaccine can be safely postponed would be eligible to enroll into the study.
Participants who have their vaccination preponed can enroll in the study only after a gap of 4 weeks following administration of the vaccine.

Leukotriene antagonists/modifiers unless participant is on a continuous treatment for at least 30 days prior to visit 1.

Initiation of allergen immunotherapy within 3 months prior to visit 1 or a plan to begin therapy or change its dose during the screening period or the randomized treatment period.

Sinus surgery within 6 months before screening (visit 1) or sinonasal surgery (see note at end of Section 5.2 for definitions of sinonasal surgery) changing the lateral wall structure of the nose making the evaluation of LMK impossible.

Participants on unstable dose of INCS spray 4 weeks prior to screening visit (visit 1) and during screening period.

Participants treated with intranasal corticosteroid drops, intranasal steroid emitting devices/stents, nasal spray using exhalation delivery system, such as XHANCET during screening period.

Participants who received SCS during screening period (between visit 1 and visit 2).

Either intravenous immunoglobulin (IVIG) therapy and/or plasmapheresis within 30 days prior to screening visit (visit 1).

### Diagnostic Assessments

Participants with any of the following results at the screening visit (visit 1):
a) Positive (or indeterminate) hepatitis B surface antigen (HBsAg) or,
b) Positive total HBcAb and a negative HBsAg confirmed by positive hepatitis B virus (HBV) DNA or,
c) Positive hepatitis C virus antibody (HCVAb) confirmed by positive HCV RNA.
   Noncompliance to Completion of the e-Diary

Participants must demonstrate at least the following for acceptable compliance: Completing the e-diary for any 4 in the 7 days immediately preceding the baseline visit (visit 2).

### Other Exclusions

Individuals accommodated in an institution because of regulatory or legal order; prisoners or participants who are legally institutionalized.

Any country-related specific regulation that would prevent the participant from entering the study.

Participant not suitable for participation, whatever the reason, as judged by the Investigator, including medical or clinical conditions or participants potentially at risk of noncompliance to study procedures.

Participants are employees of the clinical study site or other individuals directly involved in the conduct of the study, or immediate family members of such individuals (in conjunction with Section 1.61 of ICH-GCP Ordinance E6).

Any specific situation during study implementation/course that may rise ethical concerns.

Sensitivity to any of the study interventions, or components thereof, or drug or other allergy that, in the opinion of the Investigator, contraindicates participation in the study.

### Note: Definitions of Sinonasal Surgery

Endoscopic sinus surgery (ESS), include all the current procedural terminology (CPT) codes used for sinus surgery, which implies restitution of physiology and is used to create a sinus cavity opening that incorporates the natural ostium; allows adequate sinus ventilation; facilitates mucociliary clearance; facilitates instillation of topical therapies:
   ESS may include also balloon sinuplasty: endoscopic nasal surgery that uses small balloon catheters that inflate to drain the large nasal sinuses.
Or “Full ESS’ defined as complete sinus opening including anterior and posterior ethmoidectomy, maxillary antrostomy (could be large), sphenoidotomy and frontal sinusotomy in the same context as ‘full’ (e.g., Draf III) but could also include extension beyond the confines of sinuses i.e., skull base, orbit, pterygopalatine and infratemporal fossa.
   Or radical Functional endoscopic sinus surgery which includes significant removal of inflamed/dysfunctional mucosa.

### Study Intervention

Study interventions is defined as any investigations interventions marked product(s), placebo or medical device(s) intended to be administered to a participant in Part A or Part B according to the study protocol. An overview of the study interventions administered is presented in Table 18.

TABLE 18
Overview of Study Interventions Administered

| ARM name | Dupilumab | Placebo |
|---|---|---|
| Intervention name | Dupilumab 200 mg or | Placebo |
| | Dupilumab 300 mg | |
| Type | Biological | Other |
| Dose formulation | Dupilumab 200 mg: | Placebo matching dupilumab 200 mg will be |
| | A 175 mg/mL dupilumab solution | supplied as an identical formulation to the |
| | in a pre-filled syringe to | active 200 mg formulation without dupilumab, in |
| | deliver 200 mg in 1.14 mL | a pre-filled syringe to deliver placebo in |
| | or | 1.14 mL |
| | Dupilumab 300 mg: | or |
| | A 150 mg/mL dupilumab solution | Placebo matching dupilumab 300 mg will be |
| | in a pre-filled syringe to deliver | supplied as an identical formulation to the |
| | 300 mg in 2 mL | active 300 mg formulation without dupilumab, in |
| | | a pre-filed syringe to deliver placebo in 2 mL |
| Unit dose strength(s) | 200 mg or 300 mg | 0 mg |
| Dosage level(s) | One injection of 200 mg q2w for | One injection of placebo matching 200 mg q2w |
| | adolescents ≥30 kg and <60 kg at | for adolescent ≥30 kg and <60 kg at screening |
| | screening | or |
| | or | One injection of placebo matching 300 mg q2w |
| | One injection of 300 mg q2w for | for all adults and adolescents weighing ≥50 kg |
| | all adults and for adolescents | at screening |
| | weighing ≥60 kg at screening | |
| Route of administration | Subcutaneous Injection | Subcutaneous injection |
| Use | Experimental | Experimental |
| IMP | IMP | IMP |
| Packaging and labeling | Each dose of dupilumab will be | Each dose of placebo will be supplied as 1 |
| | supplied as 1 glass pre-filled | glass pre-filed syringe packed in a participant |
| | syringe packed in a participant | kit box. Both glass pre-filled syringe and box |
| | kit box. Both glass pre-filled | will be labeled as required per country |
| | syringe and box will be labeled | requirement |
| | as required per country requirement | |

Abbreviations: q2w: every 2 weeks, IMP: investigational medicinal product.

### The Investigational Medical Products (IMP)

The IMP is administered every 14±3 days (q2w) during the treatment period (with the last IMP administration at week 50 in Part A and at week 22 in Part B).

For the doses that are not scheduled to be given at the study site, home administration of IMP is allowed after appropriate training of the participant (or parent/legally authorized representative, or caregiver). Investigator or delegate will prepare and inject the first dose of IMP at visit 2, in front of the participant (or parent/legally authorized participant representative/caregiver). The participant (or parent/legally authorized representative/caregiver) will prepare and inject the IMP under the supervision of the Investigator or delegate at visit 3. The training must be documented in the participant's study file. In case of emergency (e.g., natural disaster, pandemic, etc.) different training ways (eg, training remotely with instruction provided by phone, etc.) can be performed (and will be documented in the participant's study file).

If the participant (or parent/legally authorized representative/caregiver) is unable or unwilling to prepare and inject IMP, injections can be performed at the study site by way of unscheduled visits; or arrangements can be made for qualified site personnel and/or health care professionals (e.g., visiting nurse service) to administer IMP at participant's home.

In case of emergency (e.g., natural disaster, pandemic, etc.), IMP may be supplied from the site to the participant via a Sponsor-approved courier company where allowed by local regulations and approved by the participant (or parent/legally authorized representative). When the participant has a study visit, the IMP will be administered after clinical procedures and blood collection are performed. Participants should be monitored for at least 30 minutes following injection. The monitoring period may be extended as per country-specific or local site-specific requirements.

The participant/parent/legally authorized representative/caregiver should be trained by the site staff to recognize potential signs and symptoms of hypersensitivity reaction in order to self-monitor/monitor at home for at least 30 minutes (or longer per country-specific or local site-specific requirements) following injection. In case of hypersensitivity symptoms, the participant should contact healthcare provider/emergency.

Subcutaneous injection sites should be alternated among the 4 quadrants of the abdomen (avoiding navel and waist areas), the upper thighs or the upper arms, so that the same site is not injected between two q2w injections. Injections in the upper arms could be done only by a trained person (parent/legally authorized representative/caregiver trained by Investigator or delegate) or health care professional but not the participants themselves.

For doses not given at the study site, paper diaries will be provided to the participant to record information related to the home injections. The paper diary will be kept as source data in the participant's study file.

### Background Treatments

Participants should continue their permitted background INCS spray during the study, if they were on stable dose of INCS (except for XHANCE™) for at least 4 weeks prior to screening. From the time of screening, they should not change their background medications.

### Permitted Concomitant Medication

The following treatments are allowed:
Intranasal corticosteroid spray (except for XHANCE™) is permitted as background medication that it should be on the stable dose for at least 4 weeks prior to study screening, and participants must maintain the same INCS throughout the study treatment period if initiated before enrollment.
Single dose of topical decongestants administration, for example, oxymetazoline hydrochloride (to reduce the swelling and widen the path for the endoscope) as well as a topical anesthetic (e.g., lidocaine) are allowed before nasal brushing or before endoscopy.
Administration of short courses antibiotics for a concurrent infection or CRSsNP worsening/acute sinusitis, is allowed during the study (reason for and duration of treatment should be documented in the eCRF).
Short-acting 02-adrenergic receptor agonists (SABA), long-acting f32-adrenergic receptor agonists (LABA), and long-acting muscarinic acetylcholine receptor antagonists (LAMA).
Methylxanthines (e.g., theophylline, aminophylline).
Inhaled corticosteroids.
Systemic antihistamines.
Leukotriene antagonists/modifiers are only permitted during the study, for participants who have been on a continuous treatment for ≥30 days prior to Visit 1.
Allergen immunotherapy in place for ≥3 months and dose stable for 1 month prior to visit 1 is permitted.
Short courses of SCS to treat other serious co-existing diseases (such as asthma exacerbation) or for AEs are allowed.

Other concomitant medication may be considered on a case-by-case basis by the Investigator in consultation with the Sponsor or Sponsor representative(s) if required.

### Prohibited Medications

The following concomitant treatments are not permitted during the screening period and the randomized treatment period:
Any systemic immunosuppressive treatment, such as methotrexate, cyclosporine, mycophenolate, tacrolimus, etc.
Initiation of allergen immunotherapy.
Intranasal corticosteroid drops; intranasal steroid emitting devices/stents; nasal spray using Exhalation Delivery System such as XHANCE™
All forms of SCS are prohibited during screening and study treatment period except for short-term courses (≤2 weeks) of OCS can be used as rescue during treatment period.
Live attenuated vaccines.
Intravenous immunoglobulin (IVIG) therapy.
Other monoclonal antibodies (biological immunomodulators), including but not limited to anti-IgE, anti-IL-5, and anti-tumor necrosis factor (TNF), etc.
Systemic antibiotics are prohibited during screening and study treatment period except for rescue use.

### Rescue Medicine

During the study treatment and follow-up periods, based on clinical evaluation, in case of worsening of signs and/or symptoms/acute sinusitis requiring medical intervention, the

Investigator may consider rescue treatment with:
Systemic antibiotics (up to 2 weeks) in case of acute infection.
Short course OCS (prednisone or equivalent prednisolone up to 7 days; avoid use 4 weeks before week 24 or week 52, where applicable).
Sinonasal surgery for CRSsNP (8 weeks of IMP treatment is recommended prior to surgery to allow onset of treatment effect).
Intranasal corticosteroids spray (initiation of INCS spray or change in dosing of a background INCS during the study period).

Participants receiving rescue treatment other than surgery during the study should continue on study drug unless the Investigator decides to withdraw the study treatment. Before starting treatment with OCS participants should come to the study site for the clinical assessments. It is recommended to avoid use of OCS as rescue therapy during the week 20 to week 24 (Part A and Part B) or and the week 48 to week 52 (Part A). If a participant must use OCS during these periods to control worsening of symptoms based on Investigator judgment the site should make every effort to schedule the week 24 CT scan (Part A and Part B) or and the week 52 CT scan (Part A) prior to OCS rescue therapy.

### Efficacy Assessments

### Computerized Tomography Scans Using Lund-Mackay

Computerized tomography scans using the LMK allows the assessment of sinus opacification.

The CT scan LMK staging system represents the most widely established method of sinus CT scoring in clinical trials. The LMK total score is based on assessment of the CT scan findings for each sinus area (maxillary, anterior ethmoid, posterior ethmoid, sphenoid, and frontal sinus on each side). The extent of mucosal opacification is rated on a 3-point scale ranging from 0=normal to 2=total opacification. In addition, the osteomeatal complex is graded as 0=not occluded or 2=occluded. The maximum score is therefore 12 per side for a maximum total score of 24, corresponding to the sum of all sinuses and the osteomeatal unit.

This scoring system has been validated in several studies.

The CT scans should be performed anytime during the screening period before first administration of IMP at visit 2 (baseline CT data), visit 5 and visit 7 (week 24 and week 52) for Part A and visit 5 (week 24) for Part B. Whenever possible, a cone beam CT scan should be utilized. In countries for which a specific approval procedure for the CT scan is required by a different committee than the local IEC/IRB, a 4-month window is required between 2 CT scans. If a participant needs to be rescreened or is terminated early from the study intervention, the repeated CT scan or following CT scan must be done over a 4-months restriction window. If Participant who discontinue the study intervention prematurely, the CT scan will be performed as soon as possible, with all other assessments normally planned in EOT Visit as long as between 2 CT scans can meet a 4-months restriction window. Otherwise, the CT scan can be done in next scheduled visit.

It is recommended to avoid use of OCS as rescue therapy during the week 20 to week 24 (Part A and Part B) or and the week 48 to week 52 (Part A). If a participant must use OCS during these periods to control CRSsNP worsening/acute sinusitis based on Investigator judgment the site should make every effort to schedule the week 24 CT scan (Part A and Part B) or and the week 52 CT scan (Part A) prior to OCS rescue therapy.

The CT scan will be performed locally and reviewed centrally in a pre-specified manner as described in the Image Acquisition Standards (IAS) and Image Interpretation Standards (IIS).

### CRSsNP Sinonasal Symptom Diary, Including Nasal Congestion/Obstruction, Loss of Sense of Smell, Anterior and Posterior Rhinorrhea, Facial Pain/Pressure and Headache Symptoms

The CRSsNP sinonasal symptom diary is designed to assess the severity of CRS sinonasal symptoms daily. These symptoms include NC/obstruction, anterior rhinorrhea and posterior rhinorrhea, and facial pain/pressure, and loss of smell. Each of the individual items of the diary are scored from 0 (“No symptoms”) to 3 (“Severe symptoms—symptoms that are hard to tolerate, cause interference with activities or daily living”). Higher scores on the items of the individual symptoms denote greater symptom severity.

The CRSsNP sinus Total Symptom Score (sTSS) is a composite score derived from the following individual items: NC, anterior/posterior rhinorrhea, and facial pain/pressure. The total score ranges from 0 to 9 and consists of the sum of NC, the averaged rhinorrhea item scores, and facial pain/pressure scores. Higher scores on sTSS indicate greater overall symptom severity.

The CRSsNP sinonasal symptom diary also includes an individual item about headache. Headache severity is scored on a scale ranging from 0 (“No symptoms”) to 3 (“Severe symptoms—symptoms that are hard to tolerate, cause interference with activities or daily living”). The score is not a part of sTSS.

The CRSsNP sinonasal symptom diary will be administered electronically. The e-diary is used for daily recording of participant's answers to the questionnaires. This device will be dispensed at the screening visit (visit 1), including instructions for use. Participants will be instructed on the use of the device. Recorded information will be downloaded from this device daily. At EOS Visit, the e-diary will be downloaded and returned to the site. On regular basis, the site staff should review on the vendor's website the information downloaded from participants' e-diary. They should particularly check status of the disease as well as compliance to background/rescue therapy and overall e-diary compliance. The site should follow-up with the participant as appropriate.

Participants will complete the CRSsNP sinonasal symptom diary.

### University of Pennsylvania Smell Identification Test

The UPSIT is a rapid and easy-to-administer method to quantitatively assess human olfactory function. The test consists of 4 booklets, each containing 10 odorants with one odorant per page. Above each odorant strip is a multiple-choice question with 4 alternative words to describe the odor that the participant is asked to indicate which word best describes the odor. The score ranges from 0 to 40, with 40 being the best possible score. Participant's olfactory function can be classified based on their scores. The UPSIT will be procured and used to match to each culture easing participants identifications of the odorant depending on degree of loss of sense of smell.

The clinician will administer and score the UPSIT.

### Sino-Nasal Outcome Test-22-Items

The SNOT-22 is a PRO questionnaire designed to assess the impact of CRS on patients' HRQoL. SNOT-22 has 22 items covering symptoms, social/emotional impact, productivity, and sleep consequences of CRS. The recall period is past 2 weeks. Each item is rated on a 6-point Likert scale response option, ranging from 0 (‘No problem’) to 5 (‘Problem as bad as it can be’). A global score ranging from 0 to 110 is calculated by summing the responses to all items; higher score indicates greater rhinosinusitis-related health burden. The questionnaire is an easy, valid, and reliable tool. The minimally important difference that is the smallest change in SNOT-22 score that can be detected by a participant was found to be 8.9 points.

### Rhinosinusitis Severity Visual Analog Scale

The rhinosinusitis severity VAS is used to evaluate the overall severity of the rhinosinusitis. It is a recommended scale to determine the patient's disease severity and to guide the treatment for CRS. The participant is asked to answer the following question: “How troublesome are your symptoms of your rhinosinusitis” on a 10-cm VAS from 0 (‘not troublesome’) to 10 (‘worst thinkable troublesome’). Based on their score on the VAS, the severity of rhinosinusitis can be divided into 3 categories as follows:
Mild=VAS 0 to 3
Moderate=VAS>3 to 7
Severe=VAS>7 to 10

### Asthma Control Questionnaire 6-Item Version

The asthma control questionnaire 6-items (ACQ-6) was designed to measure both the adequacy of asthma control and the change in asthma control that occurs either spontaneously or as a result of treatment. The ACQ-6 has 6 questions that assess the most common asthma symptoms: 1) Frequency in past week awoken by asthma during the night, 2) Severity of asthma symptoms in asthma, 5) Frequency of wheezing, and 6) Short-acting bronchodilator use.

Participants are asked to recall how their asthma has been during the previous week and to respond to the symptom questions on a 7-point scale (0=no impairment, 6=maximum impairment). The questions are equally weighted; and the ACQ-6 global score is the mean of the 6 questions ranging from 0 (totally controlled) to 6 (severely uncontrolled). A higher score indicates lower asthma control. On the 7-point scale of the ACQ-6, a change or difference in score of 0.5 is the smallest change that can be considered clinically important, corresponding to the minimal clinically important difference (MCID) defined by the developer. Measurement properties such as reliability and the ability to detect change have been documented in the literature.

### Patient Global Impression of Severity of CRSsNP and Patient Global Impression of Change of CRSsNP

The Patient Global Impression of Change (PGIC) is a 1-item questionnaire that asks the participant to provide the overall self-assessment of change in their CRSsNP on a 7-point scale compared to just before the participant started taking the study treatment. The response choices are: 0=“Very much better”, 1=“Moderately better”, 2=“A little better”, 3=“No change”, 4=“A little worse”, 5=“Moderately worse”, and 6=“Very much worse”.

The Patient Global Impression of Severity (PGIS) is a 1-item questionnaire that asks participants to provide the overall self-assessment of their CRSsNP severity on a 4-point scale for the past week. Response choices are: 1=“None”, 2=“Mild”, 3=“Moderate”, and 4=“Severe”.

### EuroQol-5 Dimensions Questionnaire

The EuroQol-5 dimensions (EQ-5D) is a standardized PRO measure of health status developed by the EuroQol Group in order to provide a simple, generic measure of health for clinical and economic appraisal. The adult version of the questionnaire is adapted to participants age 16 and older and can be used for participants aged 12-15 as stated in the EQ-5D user guide. The EQ-5D consists of 2 parts: the descriptive system and the EuroQol-VAS. The EQ-5D-5L descriptive system comprises the following 5 dimensions: mobility, self-care, usual activities, pain/discomfort, and anxiety/depression. Each dimension has 5 levels of perceived problems: “no problem,” “slight problems,” “moderate problems,” “severe problems,” and “extreme problems.” The respondent is asked to indicate his/her health state by ticking (or placing a cross) in the box against the most appropriate statement in each of the 5 dimensions; this results in a 1-digit number expressing the level for that dimension. The digits for 5 dimensions can be combined in a 5-digit number describing the respondent's health state.

The EQ-VAS records the respondent's self-rated health on a vertical, VAS where the endpoints are labeled “best imaginable health state (100)” and “worst imaginable health state (0)”. This information can be used as a quantitative measure of health outcome as judged by the individual respondents. The recall period of the questionnaire is “today.”

### Health Care Resource Utilization and Missed Days at Work/Missed Days at School

A questionnaire about health care resource utilization and productivity (missed days of school/workdays) will be collected by the Investigator for all participants throughout the study through eCRF.

### CRSsNP Worsening/Acute Sinusitis

The CRSsNP worsening/acute sinusitis will be assessed during the course of study and is defined as worsening of any of CRS symptoms that requires initiation of rescue therapy as assessed by Investigator.

### Spirometry

Spirometry will be performed at the local level (study site or another facility) in the morning after withholding the last dose of salbutamol/albuterol or levosalbutamol/levalbuterol for at least 6 hours and LAMA and LABA for 24 hours. The FEV1, forced vital capacity (FVC), and forced expiratory flow at 25% to 75% of forced vital capacity (FEF 25 to 75) will be determined at the designated treatment visits. The results of FEV1 (% of predicted normal), FVC and FEF 25 to 75 should be recorded in the eCRF during the screening period (before V2) for all participants and in participants with asthma for the other scheduled visits during the randomized treatment period. Whenever possible, the same spirometer and standard spirometric techniques, including calibration, will be used to perform spirometry at all visits and, the same person should perform the measurements.

### Type-2 Inflammation Transcriptomics

A gene signature representing type-2 inflammation has been curated from the literature, preclinical experiments performed at Regeneron, and dupilumab response signatures from AD and a Phase 2 study of EoE (Regeneron unpublished data). Normalized Enrichment Score (NES) reflects the degree to which the activity level of a set of transcripts is overrepresented at the extremes (top or bottom) of the entire ranked list of transcripts within a sample and is normalized by accounting for the number of transcripts in the set. The NES scores will be calculated for each transcriptome signature for each sample for statistical analyses.

### Safety Assessments

The same safety assessment was applied across both arms. Adverse events, including SAE and adverse events of special interest (AESI) were collected at every visit.

### Pharmacodynamics

Several types of samples will be collected to quantify the following PD biomarkers (optional for certain countries and sites):
Blood for total IgE, eotaxin-3, periostin, and TARC.
Nasal secretion for total IgE, eotaxin-3, IL-5, tryptase, eosinophil cationic protein, secreted P-glycoprotein, periostin, and total protein.
Nasal brushing for RNA, cell biomarkers, and eosinophils.
Urine for LTE4 and tetranor-PGDM and creatinine (collection time and date to be collected).
Serum/plasma for archival purpose (optional).

Whole blood for RNA expression (optional for adult participants). More detailed information on the collection, handling, transport, and preservation of samples (e.g., minimum volumes required for blood collection and for aliquots for each biomarker assay) will be provided in a separate laboratory manual.

Assay methodologies are briefly summarized below.

### Whole Blood Biomarkers:

Blood eosinophil count will be measured as part of the standard 5-part WBC differential cell count on a hematology autoanalyzer. Blood eosinophil count will be measured at time points additional to hematology.

### Plasma/Serum Biomarkers:

Thymus- and activation-regulated chemokine will be assayed using a validated enzyme immunoassay. Eotaxin-3 will be measured in heparinized plasma with a validated enzyme immunoassay. Total IgE will be measured with a quantitative method (e.g., Phadia ImmunoCAP) approved for diagnostic testing.

### Nasal Secretion Biomarkers:

Total IgE, tryptase and eosinophil cationic protein will be measured with a quantitative method (e.g., Phadia ImmunoCAP); periostin, eotaxin-3, IL-5, and secreted P-glycoprotein will be assayed using immunoassays.

### Genetics

Pharmacogenetic/pharmacogenomic testing from whole blood samples is optional and voluntary. Written informed consent must be obtained before sampling.

For those participants who consent to the optional pharmacogenetic/pharmacogenomic sample collection section of the ICF, blood samples for exploratory genetic analysis of DNA or RNA will be collected at the study visit and these samples will be stored for future analysis.

### Example 2. Top Line Results of an Updated Phase 2 Study Evaluating the Efficacy and Safety of Dupilumab in Patients with Uncontrolled CRSsNP—EFC16723 (ORION)

### Study Objectives and Overview

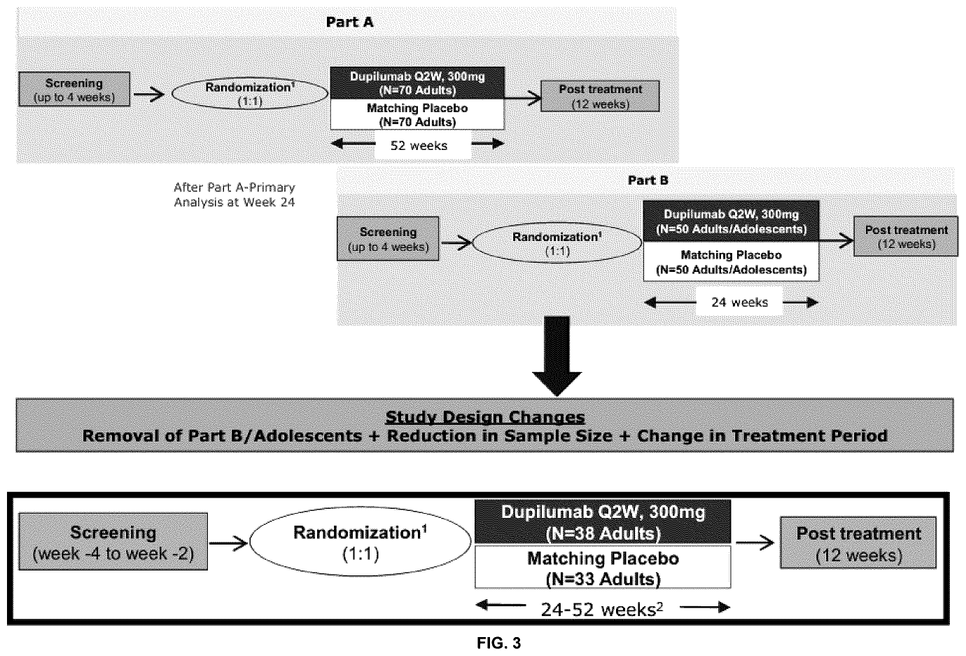

This is a Phase 2 multicenter, 1-part study to evaluate the efficacy and safety of dupilumab compared to placebo, in participants with uncontrolled CRSsNP. The trial set forth in Example 1 above was modified to remove Part B and adolescent patients, decrease sample size to n=71, and update primary endpoint to Lund-Mackay (LMK) CT score only in the dupilumab group (sTSS to secondary) and to update primary population to patients with baseline EOS levels of ≥300 cells/μL. (FIG. 3.)

### Primary Endpoint

Change in baseline LMK score at 24 weeks, dupilumab only.

### Primary Analysis Population

Screening blood EOS levels≥300 cells/μL.

### Key Inclusion Criteria

### 1. Radiographic

Bilateral inflammation of paranasal sinuses in CT scan with LMK≥8 (must include ethmoid opacification with combined score of anterior and posterior ethmoid≥1 on both sides).

### 2. Symptomatic

sTSS (composite score of NC, anterior/posterior rhinorrhea, and facial pain/pressure)≥5 (must include NC≥2; any severity of ongoing anterior/posterior rhinorrhea, with or without facial pain/pressure). Ongoing loss of smell.

### 3. Uncontrolled CRSsNP

Prior sinonasal surgery for CRS or systemic corticosteroid (SCS) treatment for CRS (any dose/duration within 2 years before screening) or SCS intolerance/contraindication.

### Key Exclusion Criteria

ppFEV1≤50% at V1.

Nasal cavity tumors.

Invasive/expansive fungal rhinosinusitis.

Endoparasitic infection/use of antiparasitic drug.

### Endpoint Assessments

### Lund-Mackay (LMK) Score

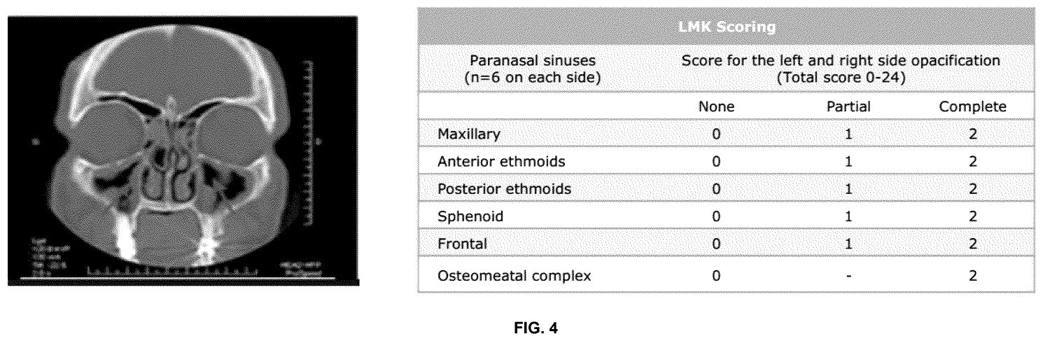

LMK was used as a primary endpoint of the dupilumab study only. LMK is a radiographic endpoint that assesses each sinus (anterior ethmoid, posterior ethmoid, maxillary, frontal, sphenoid) and OMC based on opacification. (FIG. 4.) LMK uses a scale of 0-24, wherein a higher score is worse and radiographically more opacified. The minimally clinical important differences (MCID) score for LMK is −4.

Sinus Total Symptom Score (sTSS) for CRSsNP

sTSS was used as a clinical secondary endpoint. sTSS includes facial pain/pressure instead of sense of smell, which is included in TSS, and differentiation made based on clinical picture of CRSwNP (TSS) vs. CRSsNP (sTSS). sTSS is a composite score assessing nasal congestion, anterior/posterior rhinorrhea, facial pain/pressure that uses a scale of 0-9, in which a higher is worse, and clinically more symptomatic. The MCID score for sTSS is −2.

### University of Pennsylvania Smell Identification Test (UPSIT) Score

UPSIT score was used as clinical exploratory endpoint. UPSIT assesses 40 odorants with four multiple choice answers to assess sense of smell that uses a scale of 0-40, wherein a higher score is better and indicates stronger olfactory function. The MCID score for UPSIT is 2 (Mahadev A, Kallogjeri D, Piccirillo J F. Validation of Minimal Clinically Important Difference (MCID) for University of Pennsylvania Smell Identification Test (UPSIT). Am J Rhinol Allergy. 2024 March; 38(2):123-132. doi: 10.1177/19458924231218037. Epub 2023 Dec. 6. PMID: 38055971).

### Sinonasal Outcome Test-22 (SNOT-22) Score

SNOT-22 score was used as an exploratory clinical endpoint. SNOT-22 assesses the impact of CRS on health related quality of life (HRQoL) (symptoms, social/emotional impact, productivity, sleep consequences) with a two week recall period that uses a scale of 0-110, wherein a higher score is worse and indicates a greater burden. The MCID score for UPSIT is −12 (Phillips K M, Hoehle L P, Caradonna D S, Gray S T, Sedaghat A R. Minimal clinically important difference for the 22-item Sinonasal Outcome Test in medically managed patients with chronic rhinosinusitis. Clin Otolaryngol. 2018 October; 43(5):1328-1334. doi: 10.1111/coa.13177. Epub 2018 Jul. 26. PMID: 29953729.).

### Results

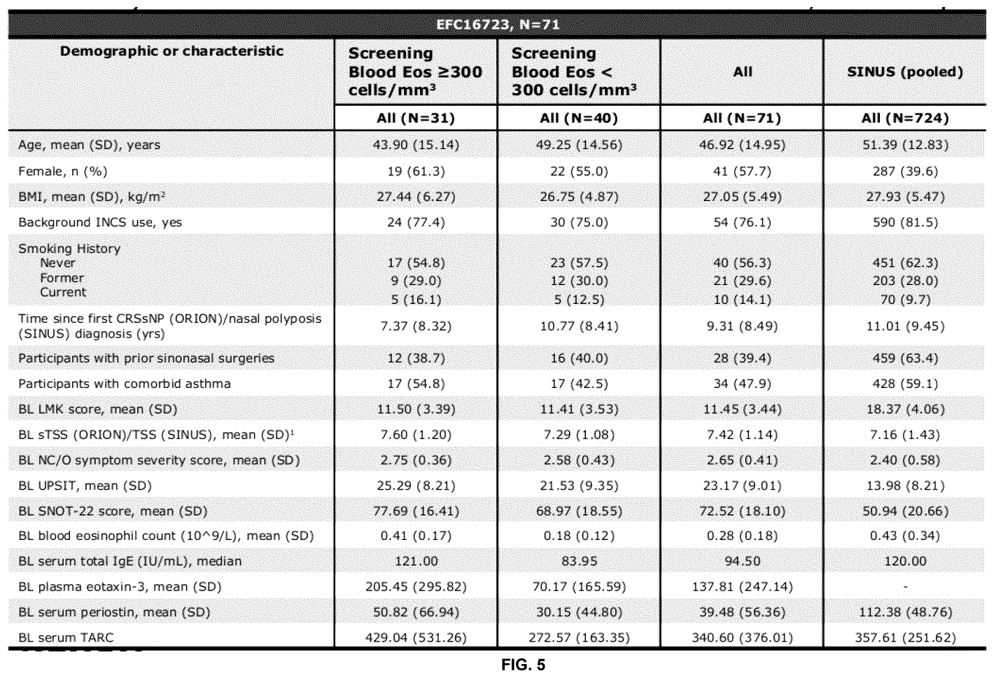

Baseline demographics, disease characteristics and biomarker data comparing CRSsNP and CRSwNP are shown at FIG. 5. The results were generally similar between the ORION (CRSsNP) and SINUS (CRSwNP) studies, with the following differences observed for CRSsNP vs. CRSwNP: 1. A lower disease severity at baseline for CRSsNP; 2. A lower LMK score at baseline for CRSsNP; 3. A higher UPSIT score at baseline for CRSsNP; 4. A lower percentage of prior surgery at baseline for CRSsNP; and 5. Lower levels of T2 biomarkers at baseline for CRSsNP.

### Endpoints

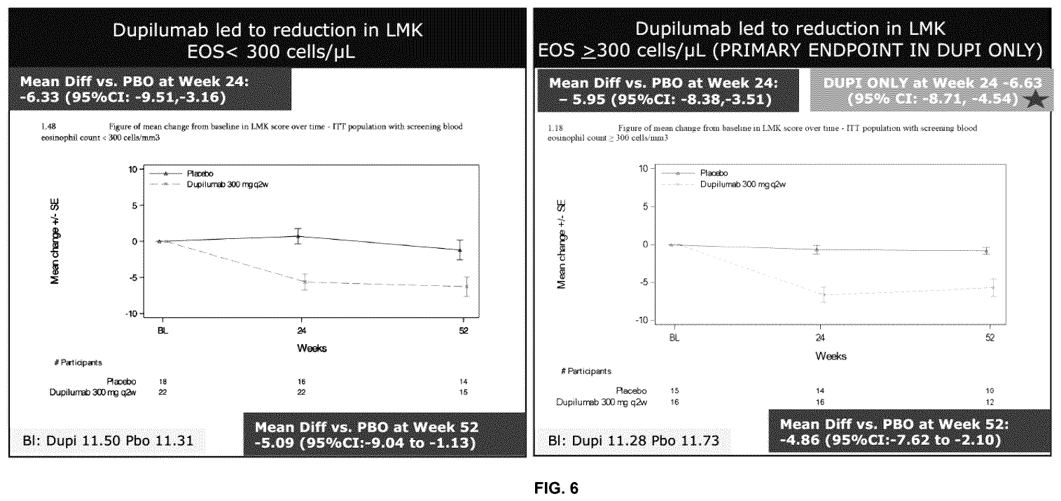

The dupilumab primary endpoint was positive and clinically meaningful. (FIG. 6.) The mean change in baseline LMK at week 24 in the dupilumab only portion of the study was positive and clinically meaningful. Similar improvements were observed when compared to placebo for both EOS≥300 and <300 cells/μL.

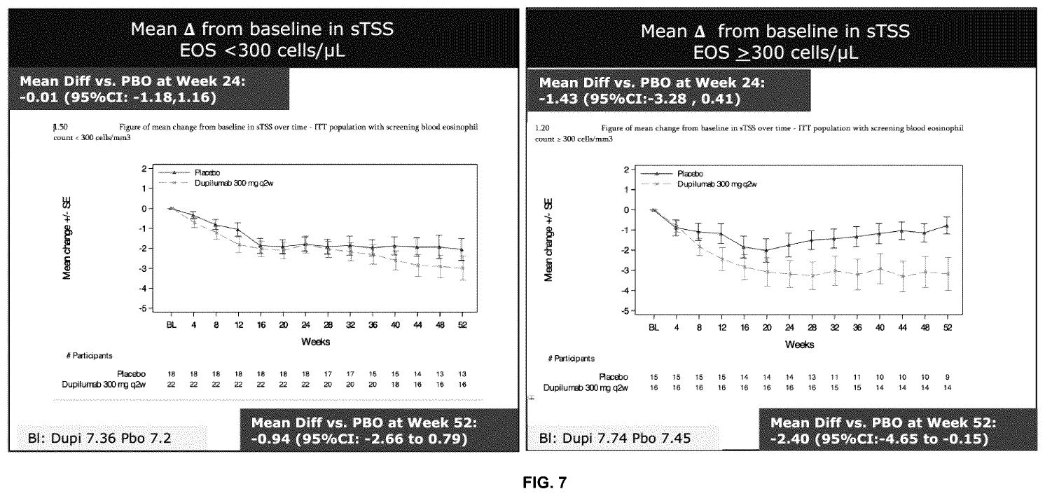

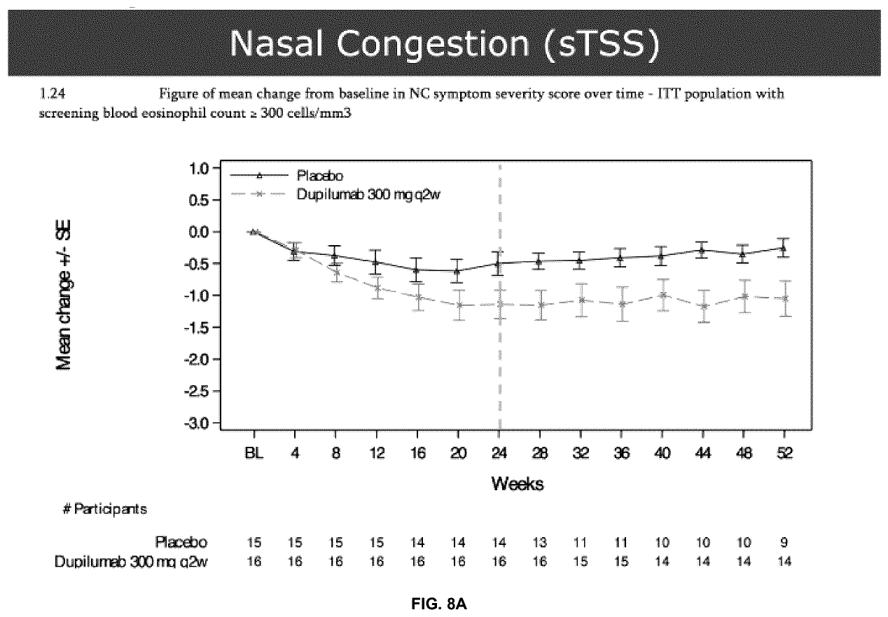

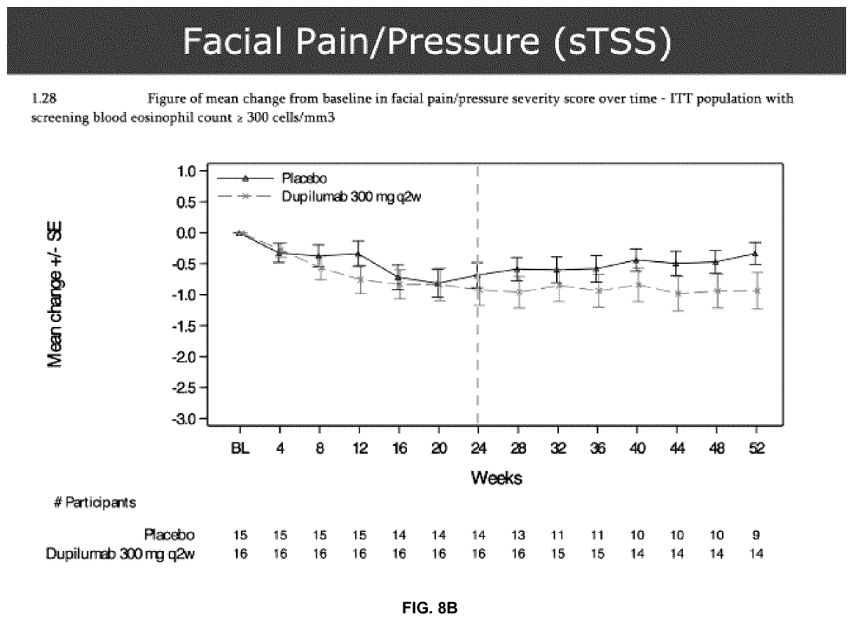

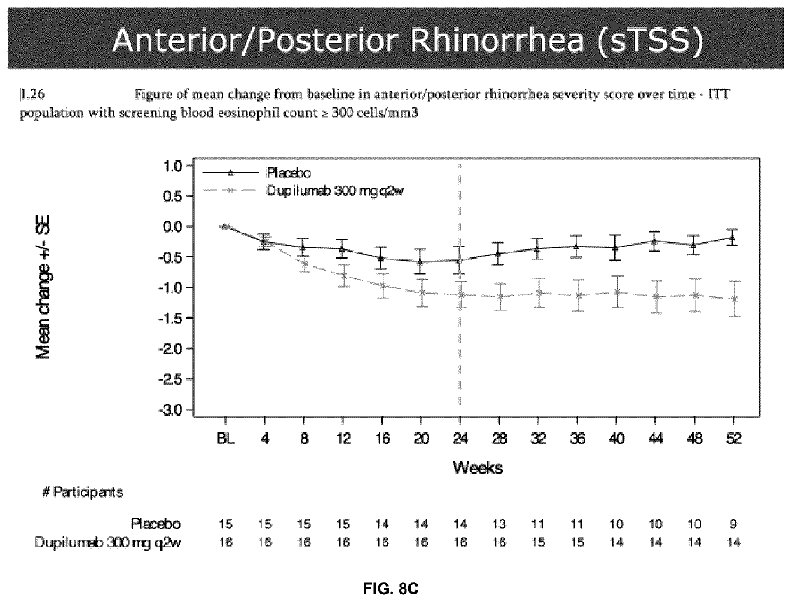

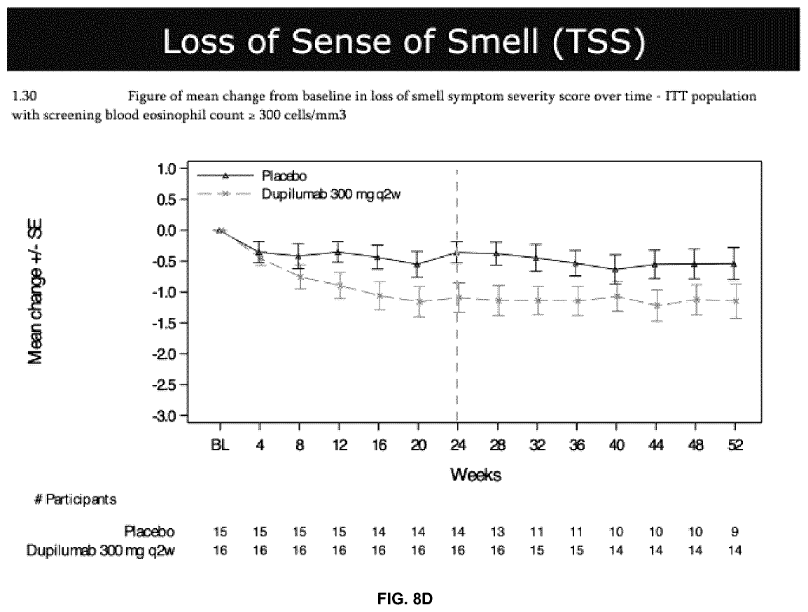

Dupilumab showed a trend to improvement in the secondary endpoint, sTSS scores, in patients with EOS levels of ≥300 cells/μL, and not in patents with EOS levels of <300 cells/μL. (FIG. 7.) An improvement was observed in sTSS/TSS scores at week 24 in CRSsNP patients with EOS levels of ≥300 cells/μL for each of: nasal congestion (FIG. 8A); facial pain/pressure (FIG. 8B); anterior/posterior rhinorrhea (FIG. 8C); and loss of sense of smell (FIG. 8D).

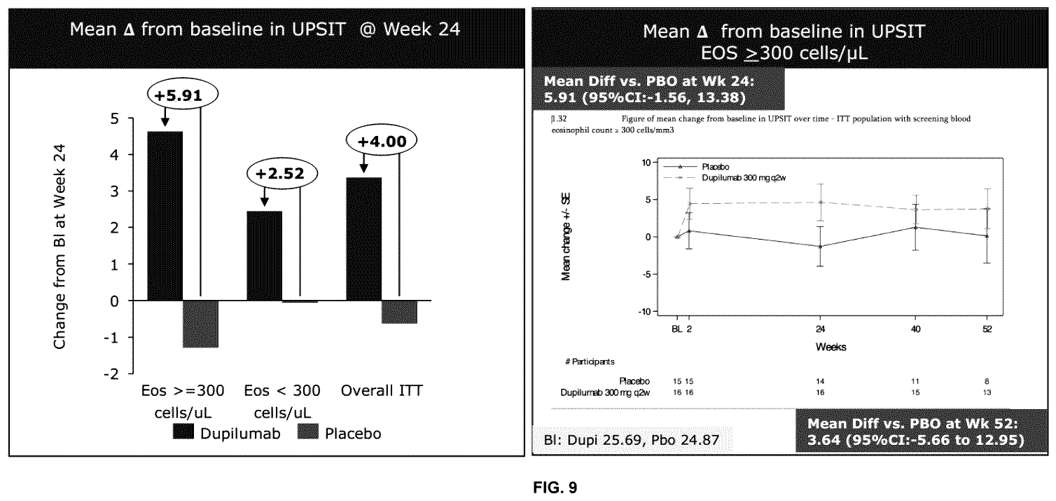

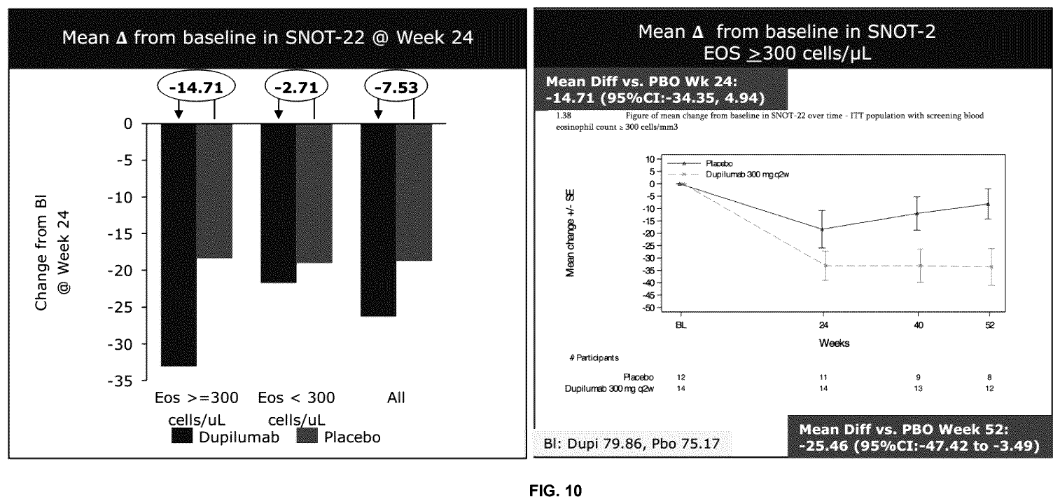

Dupilumab showed a trend to improvement in the exploratory endpoint, UPSIT scores, in patients with EOS levels of ≥300 cells/μL (FIG. 9). Dupilumab showed a trend to improvement in the exploratory endpoint, SNOT-22 scores, in patients with EOS levels of ≥300 cells/μL (FIG. 10).

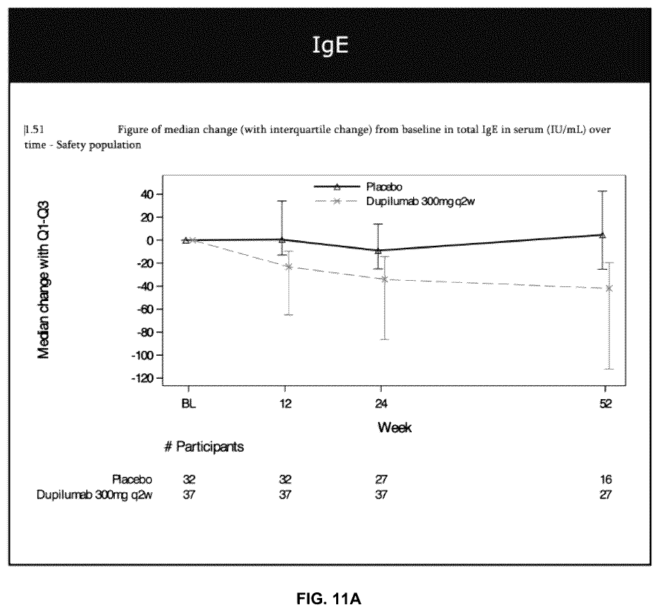

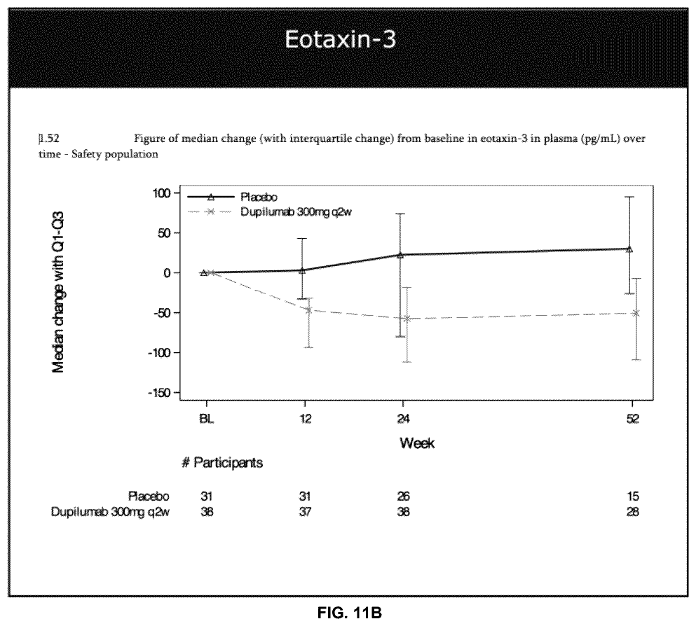

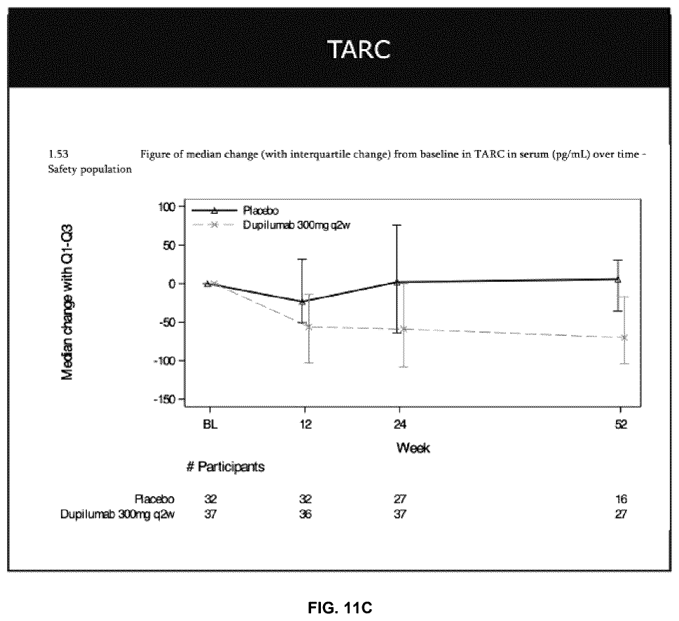

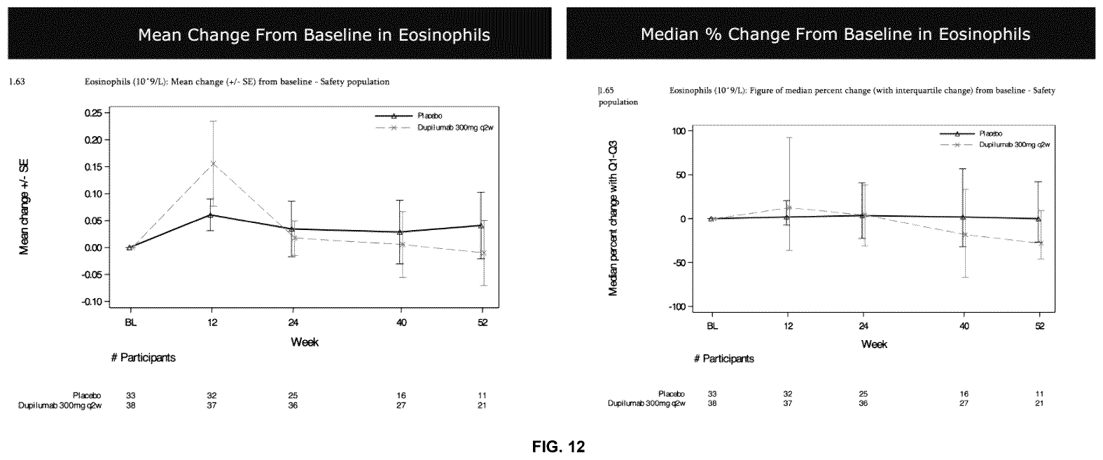

Dupilumab treatment decreased the following biomarkers of Type 2 inflammation: immunoglobulin E (IgE) (FIG. 11A); eotaxin-3 (FIG. 11B); and thymus and activation regulated chemokine (TARC) (FIG. 11C). Dupilumab treatment resulted in a transient increase in mean, but not median, eosinophils followed by return to at or below baseline (FIG. 12). No cases of clinically symptomatic eosinophilia were observed.

### Pharmacokinetics (PK) and Anti-Drug Antibody (ADA) Responses

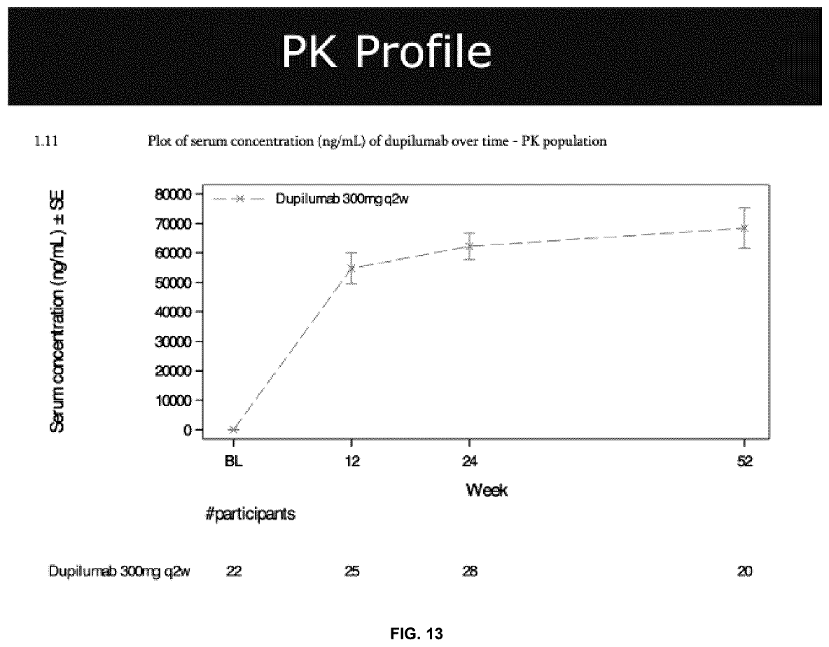

The PK profile of CRSsNP patients treated with dupilumab is shown at FIG. 13. The PK of dupilumab in patients with CRSsNP was similar to adult patients with atopic dermatitis (AD), asthma, CRSwNP, eosinophilic esophagitis (EOE), prurigo nodularis (PN), chronic spontaneous urticaria (CSU), and chronic obstructive pulmonary disease (COPD).

The ADA responses in patients with CRSsNP were consistent with observations in other indications, i.e., AD, asthma, CRSwNP, EOE, PN, CSU and COPD.

A low incidence of immunogenicity (ADA and NAb) was observed: 1 (2.6%) patient in the dupilumab arm vs. 2 (6.1%) patients in the placebo arm.

### Safety

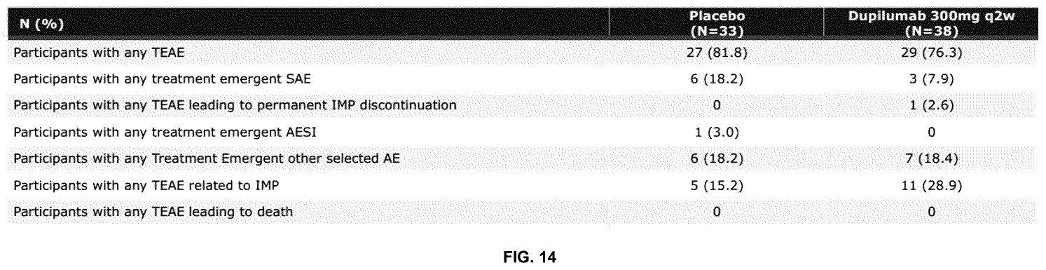

Dupilumab was well-tolerated, and no new safety concerns were identified in participants with CRSsNP. Overall, the percentage of participants with any TEAE was similar between treatment groups. (FIG. 14.) The most common TEAEs (>10% of participants in either treatment group) were COVID-19 (21.1% dupilumab and 21.2% placebo) and injection site erythema (10.5% dupilumab and 3.0% placebo).

Treatment emergent SAEs were reported in a higher percentage of participants in the placebo group compared to the dupilumab group, and TEAEs related to IMP were reported in a higher percentage of participants in the dupilumab group compared to the placebo group.

In the dupilumab group, there were no treatment emergent AESIs reported. The AE of injection site reaction occurred in a higher percentage of participants in the dupilumab group compared to the placebo group (15.8% versus 6.1%).

TEAE leading to permanent IMP discontinuation in the dupilumab group was a non-serious event of COVID-19.

There were no reports of clinically symptomatic eosinophilia or death.

### Discussion

In the phase 2 CRSsNP ORION/EFC16723 study presented in this example, the primary endpoint, change from baseline in LMK score at week 24 with dupilumab treatment only in subjects with CRSsNP having EOS levels of ≥300 cells/μL, was positive and clinically meaningful. Improvements in LMK score in dupilumab vs. placebo were observed for both EOS≥300 cells/μL and EOS<300 cells/μL populations.

A trend toward improvement in sTSS score in the dupilumab group was only observed in EOS≥300 cells/μL populations, with a larger improvement observed at week 52.

Dupilumab trend to improvement was observed in exploratory endpoints of UPSIT and SNOT-22, with benefits predominantly observed in the EOS≥300 cells/μL subpopulation.

Dupilumab treatment reduced biomarkers of type 2 inflammation.

Dupilumab was well-tolerated, and no new safety concerns were identified in participants with CRSsNP.

Overall, the percentage of participants with any TEAE was similar between treatment groups.

1. (A) method for treating a subject having chronic rhinosinusitis without nasal polyps (CRSsNP) comprising administering to the subject an antibody that specifically binds interleukin-4 receptor (IL-4R),
wherein the antibody comprises three heavy chain CDR sequences comprising SEQ ID NOs: 3, 4, and 5, respectively, and three light chain CDR sequences comprising SEQ ID NOs: 6, 7, and 8, respectively; or
(B) a method for treating a subject having uncontrolled chronic rhinosinusitis without nasal polyps (CRSsNP) comprising administering to the subject an antibody that specifically binds interleukin-4 receptor (IL-4R),
wherein the antibody comprises three heavy chain CDR sequences comprising SEQ ID NOs: 3, 4, and 5, respectively, and three light chain CDR sequences comprising SEQ ID NOs: 6, 7, and 8, respectively; or
(C) a method for treating a subject having chronic rhinosinusitis without nasal polyps (CRSsNP) with Type 2 inflammation comprising administering to the subject an antibody that specifically binds interleukin-4 receptor (IL-4R),
wherein the antibody comprises three heavy chain CDR sequences comprising SEQ ID NOs: 3, 4, and 5, respectively, and three light chain CDR sequences comprising SEQ ID NOs: 6, 7, and 8, respectively; or
(D) a method for treating a subject having chronic rhinosinusitis without nasal polyps (CRSsNP) not adequately controlled on background treatment, comprising administering to the subject an antibody that specifically binds interleukin-4 receptor (IL-4R),
wherein the antibody comprises three heavy chain CDR sequences comprising SEQ ID NOs: 3, 4, and 5, respectively, and three light chain CDR sequences comprising SEQ ID NOs: 6, 7, and 8, respectively; or
(E) a method for treating a subject having uncontrolled chronic rhinosinusitis without nasal polyps (CRSsNP) with Type 2 inflammation comprising administering to the subject an antibody that specifically binds interleukin-4 receptor (IL-4R),
wherein the antibody comprises three heavy chain CDR sequences comprising SEQ ID NOs: 3, 4, and 5, respectively, and three light chain CDR sequences comprising SEQ ID NOs: 6, 7, and 8, respectively; or
(F) a method for treating a subject having uncontrolled chronic rhinosinusitis without nasal polyps (CRSsNP), comprising selecting a subject having uncontrolled CRSsNP, and administering to the subject an antibody that specifically binds interleukin-4 receptor (IL-4R),
wherein the antibody comprises three heavy chain CDR sequences comprising SEQ ID NOs: 3, 4, and 5, respectively, and three light chain CDR sequences comprising SEQ ID NOs: 6, 7, and 8, respectively.

2-6. (canceled)

7. The method of claim 1, wherein:
the subject further has asthma, allergic rhinosinusitis, or a combination thereof;
the subject does not have asthma, allergic rhinosinusitis, or a combination thereof;
the subject has undergone endoscopic nasal surgery for CRSsNP; or
wherein the background treatment comprises inhaled corticosteroid (ICS), oral corticosteroid (OCS) or a combination thereof.

8-10. (canceled)

11. The method of claim 1, wherein the subject has one or more CRSsNP symptoms selected from the group consisting of: 1) nasal congestion/obstruction; 2) anterior rhinorrhea (runny nose); 3) posterior rhinorrhea (post nasal drip); 4) loss of sense of smell; 5) facial pain/pressure; and 6) headache, or any combination thereof.

12. The method of claim 1, wherein the subject has a blood eosinophil count of greater than or equal to about 300 cells/μL.

13. The method of claim 1, wherein the antibody is administered to the subject as an initial dose followed by one or more secondary doses, optionally wherein:
the initial dose is about 200 to about 300 mg and the one or more secondary doses are each about 200 to about 300 mg;
the initial dose is about 300 mg and the one or more secondary doses are each about 300 mg;
the initial dose is about 200 mg and the one or more secondary doses are each about 200 mg;
the secondary doses are administered every other week (q2w); or
the secondary doses are administered every four weeks (q4w).

14-18. (canceled)

19. The method of claim 1, wherein:
one or more CRSsNP symptoms are improved in the subject, optionally wherein the one or more CRSsNP symptoms include: 1) nasal congestion/obstruction; 2) anterior rhinorrhea (runny nose); 3) posterior rhinorrhea (post nasal drip); 4) loss of sense of smell; 5) facial pain/pressure; and 6) headache, or any combination thereof; or
the treatment further reduces one or more of asthma, allergic rhinosinusitis or a combination thereof, optionally wherein the treatment reduces the level of one or more biomarkers in the subject selected from the group consisting of blood eosinophil (EOS) count, serum immunoglobulin E (IgE) level, eotaxin level, thymus activation regulated chemokine (TARC) level, IL-5 level, secreted P-glycoprotein level, periostin level and combinations thereof.

20-22. (canceled)

23. The method of claim 1, wherein:
the antibody comprises a heavy chain variable region (HCVR) sequence of SEQ ID NO: 1 and a light chain variable region (LCVR) sequence of SEQ ID NO: 2; or
the antibody is dupilumab.

24. (canceled)

25. The method of claim 1, wherein the subject is at least 12 years old or wherein the subject is at least 18 years old.

26. (canceled)

27. The method of claim 1, wherein the antibody is administered subcutaneously.

28. The method of claim 1, wherein the antibody is administered using an autoinjector, a needle and syringe, or a pen; or using a prefilled device.

29. (canceled)

30. The method of claim 1, wherein the method comprises the step of determining a baseline level of a biomarker selected from the group consisting of IgE, TARC, eotaxin-3, periostin, IL-5, tryptase, eosinophil cationic protein, secreted P-glycoprotein, eosinophils, and any combination thereof in the subject.

31. A method for treating a subject having uncontrolled chronic rhinosinusitis without nasal polyps (CRSsNP), wherein the subject has a blood eosinophil level of ≥300 cells/μL, comprising administering to the subject an antibody that specifically binds interleukin-4 receptor (TL-4R),
wherein the antibody comprises three heavy chain CDR sequences comprising SEQ ID NOs: 3, 4, and 5, respectively, and three light chain CDR sequences comprising SEQ ID NOs: 6, 7, and 8, respectively.

32. The method of claim 31, wherein the subject has one or more symptoms of CRSsNP selected from the group consisting of: bilateral inflammation of paranasal sinuses; a Lund-Mackay (LMK) score of ≥8; a sinus total symptom (sTSS) score of ≥5; prior sinonasal surgery for chronic rhinosinusitis (CRS); and prior systemic corticosteroid (SCS) use for CRS.

33. The method of claim 31, wherein the antibody is administered to the subject as an initial dose followed by one or more secondary doses, optionally wherein the initial dose is about 300 mg and the one or more secondary doses are each about 300 mg and/or the antibody is administered every two weeks (Q2W).

34-35. (canceled)

36. The method of claim 31, wherein,
one or more CRSsNP symptoms are improved in the subject, optionally wherein the one or more CRSsNP symptoms are selected from the group consisting of LMK score, sTSS score, University of Pennsylvania Smell Identification Test (UPSIT) score, and Sinonasal Outcome Test-22 (SNOT-22) score; or
one or more biomarkers of Type 2 inflammation are decreased in the subject, optionally wherein the one or more biomarkers are selected from the group consisting of immunoglobulin E (IgE), eotaxin-3, and thymus activation regulated chemokine (TARC).

37-39. (canceled)

40. The method of claim 31, wherein: the antibody comprises a heavy chain variable region (HCVR) sequence of SEQ ID NO: 1 and a light chain variable region (LCVR) sequence of SEQ ID NO: 2; or the antibody is dupilumab.

41. (canceled)

42. The method of claim 1, wherein the subject is at least 18 years old.

43. The method of claim 31, wherein the antibody is administered subcutaneously.

44. The method of claim 31, wherein the antibody; is administered using an autoinjector, a needle and syringe, or a pen; or using a prefilled device.

45. (canceled)

46. The method of claim 1, wherein the uncontrolled CRSsNP is characterized by one or more of: prior sinonasal surgery for chronic rhinosinusitis; prior systemic corticosteroid use for chronic rhinosinusitis; systemic corticosteroid intolerance when previously used for chronic rhinosinusitis; and contraindication of systemic corticosteroid for previous chronic rhinosinusitis treatment, optionally wherein the systemic corticosteroid was administered to the subject at any dose and any duration within two years before treatment with the antibody.

47. (canceled)

48. A method for reducing systemic corticosteroid (SCS) use in a subject having chronic rhinosinusitis without nasal polyps (CRSsNP) comprising administering to the subject an antibody that specifically binds interleukin-4 receptor (IL-4R),
wherein the antibody comprises three heavy chain CDR sequences comprising SEQ ID NOs: 3, 4, and 5, respectively, and three light chain CDR sequences comprising SEQ ID NOs: 6, 7, and 8, respectively; or
a method for reducing oral corticosteroid (OCS) use in a subject having chronic rhinosinusitis without nasal polyps (CRSsNP) comprising administering to the subject an antibody that specifically binds interleukin-4 receptor (IL-4R),
wherein the antibody comprises three heavy chain CDR sequences comprising SEQ ID NOs: 3, 4, and 5, respectively, and three light chain CDR sequences comprising SEQ ID NOs: 6, 7, and 8, respectively.

49. (canceled)
